SEQUENCE LISTING (53 sequences)

SEQ ID NO: 1 - protein, 36 aa, Artificial Sequence
  Synthetic polypeptide
GNFWCEFWTSIDVLCVTASIETLCVIAVDRYFAITS

SEQ ID NO: 2 - protein, 42 aa, Artificial Sequence
  Synthetic polypeptide
NQAYAIASSIVSFYVPLVIMVFVYSRVFQEAKRQLQKIDKSE

SEQ ID NO: 3 - protein, 36 aa, Artificial Sequence
  Synthetic polypeptide
GSFFCELWTSVDVLCVTASIETLCVIALDRYLAITS

SEQ ID NO: 4 - protein, 40 aa, Artificial Sequence
  Synthetic polypeptide
NRAYAIASSVVSFYVPLCIMAFVYLRVFREAQKQVKKIDS

SEQ ID NO: 5 - protein, 36 aa, Artificial Sequence
  Synthetic polypeptide
GRVFCNIWAAVDVLCCTASIMGLCIISIDRYIGVSY

SEQ ID NO: 6 - protein, 42 aa, Artificial Sequence
  Synthetic polypeptide
EPGYVLFSALGSFYLPLAIILVMYCRVYVVAKRESRGLKSGL

SEQ ID NO: 7 - protein, 36 aa, Artificial Sequence
  Synthetic polypeptide
GKVWCEIYLALDVLFCTSSIVHLCAISLDRYWSITQ

SEQ ID NO: 8 - protein, 41 aa, Artificial Sequence
  Synthetic polypeptide
QKWYVISSSIGSFFAPCLIMILVYVRIYQIAKRRTRVPPSR

SEQ ID NO: 9 - protein, 36 aa, Artificial Sequence
  Synthetic polypeptide
GQVTCDLFIALDVLCCTSSILHLCAIALDRYWAITD

SEQ ID NO: 10 - protein, 42 aa, Artificial Sequence
  Synthetic polypeptide
DHGYTIYSTFGAFYIPLLLMLVLYGRIFRAARFRIRKTVKKV

SEQ ID NO: 11 - protein, 36 aa, Artificial Sequence
  Synthetic polypeptide
GPVVCDLWLALDYVVSNASVMNLLIISFDRYFCVTK

SEQ ID NO: 12 - protein, 41 aa, Artificial Sequence
  Synthetic polypeptide
NAAVTFGTAIAAFYLPVIIMTVLYWHISRASKSRIKKDKKE

SEQ ID NO: 13 - protein, 36 aa, Artificial Sequence
  Synthetic polypeptide
GNLACDLWLAIDYVASNASVMNLLVISFDRYFSITR

SEQ ID NO: 14 - protein, 41 aa, Artificial Sequence
  Synthetic polypeptide
EPTITFGTAIAAFYMPVTIMTILYWRIYKETEKRTKELAGL

SEQ ID NO: 15 - protein, 36 aa, Artificial Sequence
  Synthetic polypeptide
SRIHCDIFVTLDVMMCTASILNLCAISIDRYTAVAM

SEQ ID NO: 16 - protein, 41 aa, Artificial Sequence
  Synthetic polypeptide
NPAFVVYSSIVSFYVPFIVTLLVYIKIYIVLRRRRKRVNTK

SEQ ID NO: 17 - protein, 36 aa, Artificial Sequence
  Synthetic polypeptide
GDAGCRGYYFLRDACTYATALNVASLSVERYLAICH

SEQ ID NO: 18 - protein, 45 aa, Artificial Sequence
  Synthetic polypeptide
TATVKVVIQVNTFMSFIFPMVVISVLNTIIANKLTVMVRQAAEQG

SEQ ID NO: 19 - protein, 36 aa, Artificial Sequence
  Synthetic polypeptide
GELLCKAVLSIDYYNMFTSIFTLTMMSVDRYIAVCH

SEQ ID NO: 20 - protein, 38 aa, Artificial Sequence
  Synthetic polypeptide
SWYWDTVTKICVFLFAFVVPILIITVCYGLMLLRLRSV

SEQ ID NO: 21 - protein, 36 aa, Artificial Sequence
  Synthetic polypeptide
GDVLCKIVISIDYYNMFTSIFTLTMMSVDRYIAVCH

SEQ ID NO: 22 - protein, 38 aa, Artificial Sequence
  Synthetic polypeptide
YSWWDLFMKICVFIFAFVIPVLIIIVCYTLMILRLKSV

SEQ ID NO: 23 - protein, 36 aa, Artificial Sequence
  Synthetic polypeptide
GTILCKIVISIDYYNMFTSIFTLCTMSVDRYIAVCH

SEQ ID NO: 24 - protein, 38 aa, Artificial Sequence
  Synthetic polypeptide
TWYWENLLKICVFIFAFIMPVLIITVCYGLMILRLKSV

SEQ ID NO: 25 - protein, 36 aa, Artificial Sequence
  Synthetic polypeptide
GEALCNVLIGFFYGNMYCSILFMTCLSVQRYWVIVN

SEQ ID NO: 26 - protein, 38 aa, Artificial Sequence
  Synthetic polypeptide
LVGDMFNYFLSLAIGVFLFPAFLTASAYVLMIRMLRSS

SEQ ID NO: 27 - protein, 20 aa, Artificial Sequence
  Synthetic polypeptide
TASIETLCVIAVDRYFAITS

SEQ ID NO: 28 - protein, 23 aa, Artificial Sequence
  Synthetic polypeptide
NQAYAIASSIVSFYVPLVIMVFV

SEQ ID NO: 29 - protein, 20 aa, Artificial Sequence
  Synthetic polypeptide
TASIETLCVIALDRYLAITS

SEQ ID NO: 30 - protein, 22 aa, Artificial Sequence
  Synthetic polypeptide
NRAYAIASSVVSFYVPLCIMAF

SEQ ID NO: 31 - protein, 20 aa, Artificial Sequence
  Synthetic polypeptide
TASIMGLCIISIDRYIGVSY

SEQ ID NO: 32 - protein, 22 aa, Artificial Sequence
  Synthetic polypeptide
EPGYVLFSALGSFYLPLAIILV

SEQ ID NO: 33 - protein, 20 aa, Artificial Sequence
  Synthetic polypeptide
TSSIVHLCAISLDRYWSITQ

SEQ ID NO: 34 - protein, 22 aa, Artificial Sequence
  Synthetic polypeptide
QKWYVISSSIGSFFAPCLIMIL

SEQ ID NO: 35 - protein, 20 aa, Artificial Sequence
  Synthetic polypeptide
TSSILHLCAIALDRYWAITD

SEQ ID NO: 36 - protein, 22 aa, Artificial Sequence
  Synthetic polypeptide
DHGYTIYSTFGAFYIPLLLMLV

SEQ ID NO: 37 - protein, 20 aa, Artificial Sequence
  Synthetic polypeptide
NASVMNLLIISFDRYFCVTK

SEQ ID NO: 38 - protein, 22 aa, Artificial Sequence
  Synthetic polypeptide
NAAVTFGTAIAAFYLPVIIMTV

SEQ ID NO: 39 - protein, 20 aa, Artificial Sequence
  Synthetic polypeptide
NASVMNLLVISFDRYFSITR

SEQ ID NO: 40 - protein, 22 aa, Artificial Sequence
  Synthetic polypeptide
EPTITFGTAIAAFYMPVTIMTI

SEQ ID NO: 41 - protein, 20 aa, Artificial Sequence
  Synthetic polypeptide
TASILNLCAISIDRYTAVAM

SEQ ID NO: 42 - protein, 22 aa, Artificial Sequence
  Synthetic polypeptide
NPAFVVYSSIVSFYVPFIVTLL

SEQ ID NO: 43 - protein, 20 aa, Artificial Sequence
  Synthetic polypeptide
YATALNVASLSVERYLAICH

SEQ ID NO: 44 - protein, 25 aa, Artificial Sequence
  Synthetic polypeptide
TATVKVVIQVNTFMSFIFPMVVISV

SEQ ID NO: 45 - protein, 20 aa, Artificial Sequence
  Synthetic polypeptide
FTSIFTLTMMSVDRYIAVCH

SEQ ID NO: 46 - protein, 26 aa, Artificial Sequence
  Synthetic polypeptide
SWYWDTVTKICVFLFAFVVPILIITV

SEQ ID NO: 47 - protein, 20 aa, Artificial Sequence
  Synthetic polypeptide
FTSIFTLTMMSVDRYIAVCH

SEQ ID NO: 48 - protein, 26 aa, Artificial Sequence
  Synthetic polypeptide
YSWWDLFMKICVFIFAFVIPVLIIIV

SEQ ID NO: 49 - protein, 20 aa, Artificial Sequence
  Synthetic polypeptide
FTSIFTLCTMSVDRYIAVCH

SEQ ID NO: 50 - protein, 26 aa, Artificial Sequence
  Synthetic polypeptide
TWYWENLLKICVFIFAFIMPVLIITV

SEQ ID NO: 51 - protein, 20 aa, Artificial Sequence
  Synthetic polypeptide
YCSILFMTCLSVQRYWVIVN

SEQ ID NO: 52 - protein, 26 aa, Artificial Sequence
  Synthetic polypeptide
LVGDMFNYFLSLAIGVFLFPAFLTAS

SEQ ID NO: 53 - protein, 413 aa, Artificial Sequence
  Synthetic polypeptide
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

(19) United States
(12) Patent Application Publication
(10) Pub. No.: US 2021/0353626 A1
(43) Pub. Date: Nov. 18, 2021

(54) BETA-ADRENERGIC RECEPTOR ALLOSTERIC MODULATORS

(71) Applicants: THE REGENTS OF THE UNIVERSITY OF CALIFORNIA, Oakland, CA (US); FRIEDRICH-ALEXANDER-UNIVERSITÄT ERLANGEN-NÜRNBERG, Erlangen (DE); THE BOARD OF TRUSTEES OF THE LELAND STANFORD JUNIOR UNIVERSITY, Stanford, CA (US)

(72) Inventors: Roger K. SUNAHARA, San Diego, CA (US); Mary J. CLARK, San Diego, CA (US); Brian K. KOBILKA, Stanford, CA (US); Cheng ZHANG, Pittsburgh, PA (US); Xiangyu LIU, Beijing (CN); Peter GMEINER, Erlangen (DE); Anne STÖSSEL, Erlangen (DE); Harald HÜBNER, Erlangen (DE); Daniela DENGLER, Erlangen (DE); Markus STANEK, Erlangen (DE); Brian S. SHOICHET, San Francisco, CA (US); Magdalena KORCZYNSKA, San Francisco, CA (US); Jacob P. MAHONEY, Stanford, CA (US); Jonas KAINDL, Erlangen (DE)

(21) Appl. No.: 17/048,998

(22) PCT Filed: Apr. 19, 2019
(86) PCT No.: PCT/US19/28379

§ 371 Date: Oct. 19, 2020

Related U.S. Application Data
(60) Provisional application No. 62/660,832, filed on Apr. 20, 2018.

(51) Int. Cl. A61K 31/517; A61K 45/06; C07D 239/84; C07D 403/12; C07D 401/12

(52) U.S. Cl. CPC A61K 31/517; A61K 45/06; C07D 401/12; C07D 403/12; C07D 239/84

(57) ABSTRACT

Provided herein are modulators of beta-adrenergic receptors.

### CROSS-REFERENCES TO RELATED APPLICATIONS

This application claims the benefit of U.S. Provisional Application No. 62/660,832, filed Apr. 20, 2018, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. GM106990 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-607001WO_Sequence_Listing_ST25.txt, created Apr. 18, 2019, 23,781 bytes, machine format IBM-PC, MS Windows operating system, is hereby incorporated by reference.

### BACKGROUND

G protein-coupled receptor (GPCR)-based drug discovery has traditionally focused on targeting the binding site of native hormones and neurotransmitters. (Keov, P. et al, Neuropharmacology 2011, 60, 24-35) These orthosteric binding pockets of GPCRs, such as the family of adrenergic receptors (ARs), share a high degree of amino acid identity. As a consequence, some endogenous ligands like adrenaline and noradrenaline are recognized by all AR subtypes. This lack of subtype selectivity is also observed for pharmaceutical small molecules leading to possibly adverse off-target effects.

However, allosteric modulators do not suffer from this subtype promiscuity as their site of interaction is distinct from the highly conserved orthosteric site. The saturability of action inherent to allosteric modulators allows to fine-tune receptor signaling, thereby minimizing risks like drug overdosing. (Congreve, M. et al, Trends Pharmacol. Sci. 2017, 9, 837-847) Hence, the search for allosteric drug scaffolds offers opportunities for therapeutic use.

The β adrenergic receptors (βARs) are crucially involved in several diseases like asthma, Parkinson's disease, hypertension and heart failure. Therefore, there is a need in the art for modulators of β adrenergic receptors. Disclosed herein, inter alia, are solutions to these and other problems in the art.

### BRIEF SUMMARY

In an aspect is provided a compound having the formula:

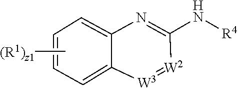

wherein R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl; z1 is an integer from 0 to 4; W² is N, CH, or C(R²); R² is independently halogen, —CX²₃, —CHX²₂, —CH₂X², —OCX²₃, —OCH₂X², —OCHX¹₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; W³ is N, CH, or C(R³); R³ is independently halogen, —CX³₃, —CHX³₂, —CH₂X³, —OCX³₃, —OCH₂X³, —OCHX³₂, —CN, —SOₙ₁R³⁰, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is independently substituted or unsubstituted aryl, substituted or unsubstituted heteroaryl, substituted or unsubstituted cycloalkyl, substituted or unsubstituted spirocycloalkyl, substituted or unsubstituted heterocycloalkyl, hydrogen, substituted or unsubstituted heteroalkyl, or substituted or unsubstituted alkyl; R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; and R^{3A} and R^{3B} substituents bonded to the same nitrogen atom may optionally be joined to form a substituted or unsubstituted heterocycloalkyl or substituted or unsubstituted heteroaryl; X, X¹, X², and X³ are independently —F, —Cl, —Br, or —I; n1, n2, and n3 are independently an integer from 0 to 4; and m1, m2, m3, v1, v2, and v3 are independently 1 or 2.

### BRIEF DESCRIPTION OF THE DRAWINGS

FIGS. 1A-1C. Hit to lead optimization, pharmacological characterization and structure of allosteric modulator AS408 bound to β₂AR. (FIG. 1A-1B) Hit-to-lead optimization of the docking hit BRAC1. Negative allosteric effect of BRAC1 and AS408: on norepinephrine (NE)-stimulated β-arrestin 2 recruitment; on cAMP accumulation. (FIG. 1C) Structure of AS408 bound to β₂AR in the presence of antagonist alprenolol.

FIGS. 2A-2G. Structural basis of the negative allosteric activity of AS408 on agonist binding to β₂AR. Structure of AS408 bound to β₂AR in the presence of alprenolol determined by x-ray crystallography. (FIG. 2A) illustrates residues within 3 Å of AS408 in the presence of alprenolol. (FIG. 2B, FIG. 2C) Superposition of the structures of the inactive form of β₂AR in the presence of carazolol (PDB: 2RH1) on the AS408-β₂AR structure. (FIG. 2C) Influence of AS408 on the water network formed by E122^{3.41}, S207^{5.46} & V206^{5.45}. (FIG. 2D-FIG. 2G) Comparison of the AS408-bound β₂AR structure with the active, agonist-bound β₂AR (PDB: 4LDO, green). (FIG. 2D) Positions of the side-chain of residues coordinating AS408 binding differ in the active, agonist-bound conformation. (FIG. 2E) Illustration of the capacity of AS408 to prevent the catechol ring of epinephrine to bind to S207^{5.46} (and S203^{5.42}, not shown) in TM5 and therefore prevent transition to the active conformation. (FIG. 2F, FIG. 2G) Loss of interaction of P211^{5.50} and AS408 in the agonist-bound active conformation.

FIGS. 3A-3D. Negative allosteric activity of AS408 on agonist-mediated β-arrestin 2 recruitment to β₂AR. (FIG. 3A-FIG. 3D) Varying concentration of AS408 on dose response curves for full agonists (FIG. 3A) norepinephrine, (FIG. 3B) epinephrine, (FIG. 3C) isoproterenol, or (FIG. 3D) partial agonist salmeterol. Note AS408 appears to have no positive intrinsic effect on β-arrestin recruitment on its own.

FIGS. 4A-4G. FIG. 4A shows that AS408 enhances the β₂AR affinity for the inverse agonist ICI118551. FIG. 4B shows that AS408 reduces the β₂AR affinity for the agonist norepinephrine. FIG. 4C shows that AS408 reduces affinity of agonist for uncoupled β₂AR more so than for Gs-coupled β₂AR. FIG. 4D shows that AS408 enhances the inhibition of basal activity by ICI118551. FIG. 4E shows that AS408 has weak inverse agonist activity by itself.

FIG. 4F shows that AS408 had no effect on the dissociation rate of ³H-formoterol in Gs-coupled β₂AR. FIG. 4G shows that AS408 accelerated the dissociation rate of ³H-formoterol from uncoupled β₂AR in the presence of GTPγS.

FIGS. 5A-5P. FIG. 5A. Expression of E122x mutants β₂AR in HEK cells. [³H]formoterol (agonist) binding to E122 mutants of β₂AR expressed HEK293 cells, as a fraction of total receptor (fraction of [³H]DHAP binding), or measured with antagonist binding ([³H]CGP12177). FIGS. 5B-D show the effect of AS408 binding site mutants. FIGS. 5E-G shows the effect of AS408 binding pocket mutations on cAMP and ligand binding. FIGS. 5H-J show the effect of E122A, E122F, E122K, E122Q, E122W, and E122L. FIGS. 5K-M show AS408 effect on [³H]DHAP binding. FIG. 5N-P show effect of mutation and AS408 on saturation binding by DHAP and GTPγS.

FIGS. 6A-6C. Structure of the AS408 binding site. (FIG. 6A) Fo-Fc simulated annealing omit map (contoured at 2.3 σ) reveals the binding site of AS408 in the AS408-β₂AR complex in the presence of alprenolol. (FIG. 6B) Anomalous signal (contoured at 4.0 σ) of the bromine atom at C₆ of AS408 yields a unique density corresponding to the AS408 model in (FIG. 6A) (FIG. 6C) The bromine moiety of AS408 forms a crystal contact with L45^{1.44} of a neighboring β₂AR in the crystal lattice.

FIGS. 7A-7E. Binding mode of AS408 stable in molecular dynamics (MD) simulation at the β₂AR in complex with alprenolol. (FIG. 7A) RMSD of AS408 showing that AS408 maintains a binding mode comparable to its crystallographic pose. (FIG. 7B) The primary amine of AS408 stays within hydrogen bonding distance of the carboxylate of E122^{3.41} and the carbonyl oxygen of V206^{5.45}. (FIG. 7C) The bromine substituent of AS408 maintains it's the van der Waals interaction to the side chains of V206^{5.45} and V210^{5.49}, despite being influenced by a second β₂AR protomer in the crystal structure. (FIG. 7D) The unsubstituted phenyl ring of AS408 maintains its position between the side chains of C125^{3.44}, V126^{3.45}, V129^{3.48} and I214^{5.53}. (FIG. 7E) Representative, energy minimized snapshot of the MD simulation of AS408 bound to β₂AR superimposed with the crystal structure of inactive β₂AR in complex with alprenolol and AS408.

FIGS. 8A-8B. AS408 reverses norepinephrine inhibition of [³H]DHAP binding to β₂AR in detergent micelles or in lipid. AS408 reversed 10 μM norepinephrine inhibition [³H]DHAP binding β₂AR in (FIG. 8A) dodecylmaltoside (DDM) micelles or reconstituted in high density lipoprotein particles (rHDL or nanodiscs, log(EC₅₀)˜ 5.1+/−0.09 μM and 5.2+/−0.14 μM, respectively) or (FIG. 8B) in rHDL in the absence or presence of cholesterol. (log(EC₅₀)˜6.2+/−0.08 μM and 6.1+/−0.05 μM, respectively).

FIG. 9A-9D. Structure activity relationship of AS408 analogs as a NAM for norepinephrine-stimulated β-arrestin 2 recruitment. Dose response relationships of norepineprine-stimulated β-arrestin recruitment by analogs of BRAC1 highlighting the evolution of NAM activity toward AS408. BRAC1 analogs were tested at 10 μM and 30 μM concentrations compared to norepinephrine alone. Highlighted in bold is the structure of the bromine-substituted phenyl ring of AS408. The primary amino group of protonated AS408 forms an ionic interaction with the side chain of E122^{3.41} and a hydrogen bond with the backbone oxygen of V206^{5.45} (FIG. 2C). DD288, missing the amino function, can no longer replace the mediating water molecule linking E122^{3.41} and V206^{5.45} and S207^{5.46} resulting in an attenuated negative allosteric effect. The stronger allosteric effect of AS408, compared to the initial hit (BRAC1), can be explained by attractive interactions of the bromine-substituent with the highly hydrophobic lipid-protein interface. The halogen atom fits nicely between the side chains of V206^{5.45} and V210^{5.49}, when the bromine is located in position 6. In contrast, a bromine-substituent in position 5, 7 or 8, of the heterocyclic ring led to reduced allosteric modulation, as a result of a less complementary shape or a clash with V206^{5.45}. The extent of the hydrophobic interaction to V206^{5.45} and V210^{5.49} increases with the size of the (pseudo)halogen substituent (F<<Cl<CF₃<Br<I). Further increasing the hydrophobic substituent by introduction of a phenyl group results in partial disruption of the negative allosteric effect, suggesting repulsive interactions with the side chain of V206^{5.45}. The fit of the phenyl ring of AS408 fits into a complementary hydrophobic pocket formed by C125^{3.44}, V126^{3.45}, V129^{3.48} and I214^{5.53} explains that replacement of the phenyl group by a smaller aliphatic propyl chain reduces the hydrophobic interactions and abolishes the negative allosteric effect. Loss of the allosteric effect was also observed when we introduced a hydroxyl group to the phenyl ring, which may inflict repulsive interactions at the hydrophobic membrane protein interface.

FIGS. 10A-10Q. β-adrenergic receptor selectivity of AS408. (FIG. 10A) Sequence alignment of residues in TM3 and TM5 involved in AS408 binding from various Family A GPCRs. FIG. 10A includes the following sequences: Portion of human β₂AR GNFWCEFWTSIDVLCVTASIETLCVIAVDRYFAITS (SEQ ID NO:1); Portion of human β₂AR NQAYAIASSIVSFYVPLVIMVFVYSRVFQEAKRQLQKIDKSE (SEQ ID NO:2); Portion of mouse β1AR GSFFCELWTSVDVLCVTASIETLCVIALDRYLAITS (SEQ ID NO:3); Portion of mouse β1ARNRAYAIASSVVSFYVPLCIMAFVYLRVFREAQKQVKKIDS (SEQ ID NO:4); Portion of human α1AR GRVFCNIWAAVDVLCCTASIMGLCIISIDRYIGVSY (SEQ ID NO:5); Portion of human α1AR EPGYVLFSALGSFYLPLAIILVMYCRVYVVAKRESRGLKSGL (SEQ ID NO:6); Portion of mouse α₂AR GKVWCEIYLALDVLFCTSSIVHLCAISLDRYWSITQ (SEQ ID NO:7); Portion of mouse α₂AR QKWYVISSSIGSFFAPCLIMILVYVRIYQIAKRRTRVPPSR (SEQ ID NO:8); Portion of human 5HT1AR GQVTCDLFIALDVLCCTSSILHLCAIALDRYWAITD (SEQ ID NO:9); Portion of human 5HT1AR DHGYTIYSTFGAFYIPLLLMLVLYGRIFRAARFRIRKTVKKV (SEQ ID NO:10); Portion of human M2R GPVVCDLWLALDYVVSNASVMNLLIISFDRYFCVTK (SEQ ID NO:11); Portion of human M2R NAAVTFGTAIAAFYLPVIIMTVLYWHISRASKSRIKKDKKE (SEQ ID NO:12); Portion of human M3R GNLACDLWLAIDYVASNASVMNLLVISFDRYFSITR (SEQ ID NO:13); Portion of human M3R EPTITFGTAIAAFYMPVTIMTILYWRIYKETEKRTKELAGL (SEQ ID NO:14); Portion of human D2R SRIHCDIFVTLDVMMCTASILNLCAISIDRYTAVAM (SEQ ID NO:15); Portion of human D2R NPAFVVYSSIVSFYVPFIVTLLVYIKIYIVLRRRRKRVNTK (SEQ ID NO:16); Portion of human NTS1R GDAGCRGYYFLRDACTYATALNVASLSVERYLAICH (SEQ ID NO:17); Portion of human NTS1R TATVKVVIQVNTFMSFIFPMVVISVLNTIIANKLTVMVRQAAEQG (SEQ ID NO:18); Portion of human δOR GELLCKAVLSIDYYNMFTSIFTLTMMSVDRYIAVCH (SEQ ID NO:19); Portion of human δOR SWYWDTVTKICVFLFAFVVPILIITVCYGLMLLRLRSV (SEQ ID NO:20); Portion of human κOR GDVLCKIVISIDYYNMFTSIFTLTMMSVDRYIAVCH (SEQ ID NO:21); Portion of human κOR YSWWDLFMKICVFIFAFVIPVLIIIVCYTLMILRLKSV (SEQ ID NO:22); Portion of human μOR GTILCKIVISIDYYNMFTSIFTLCTMSVDRYIAVCH (SEQ ID NO:23); Portion of human μOR TWYWENLLKICVFIFAFIMPVLIITVCYGLMILRLKSV (SEQ ID NO:24); Portion of human PAR2 GEALCNVLIGFFYGNMYCSILFMTCLSVQRYWVIVN (SEQ ID NO:25); Portion of human PAR2 LVGDMFNYFLSLAIGVFLFPAFLTASAYVLMIRMLRSS (SEQ ID NO:26); Portion of human β₂AR TASIETLCVIAVDRYFAITS (SEQ ID NO:27); Portion of human β₂AR NQAYAIASSIVSFYVPLVIMVFV (SEQ ID NO:28); Portion of mouse β1AR TASIETLCVIALDRYLAITS (SEQ ID NO:29); Portion of mouse β1AR NRAYAIASSVVSFYVPLCIMAF (SEQ ID NO:30); Portion of human α1AR TASIMGLCIISIDRYIGVSY (SEQ ID NO:31); Portion of human α1AR EPGYVLFSALGSFYLPLAIILV (SEQ ID NO:32); Portion of mouse α₂AR TSSIVHLCAISLDRYWSITQ (SEQ ID NO:33); Portion of mouse α₂AR QKWYVISSSIGSFFAPCLIMIL (SEQ ID NO:34); Portion of human 5HT1AR TSSILHLCAIALDRYWAITD (SEQ ID NO:35); Portion of human 5HT1AR DHGYTIYSTFGAFYIPLLLMLV (SEQ ID NO:36); Portion of human M2R NASVMNLLIISFDRYFCVTK (SEQ ID NO:37); Portion of human M2R NAAVTFGTAIAAFYLPVIIMTV (SEQ ID NO:38); Portion of human M3R NASVMNLLVISFDRYFSITR (SEQ ID NO:39); Portion of human M3R EPTITFGTAIAAFYMPVTIMTI (SEQ ID NO:40); Portion of human D2R TASILNLCAISIDRYTAVAM (SEQ ID NO:41); Portion of human D2R NPAFVVYSSIVSFYVPFIVTLL (SEQ ID NO:42); Portion of human NTS1R YATALNVASLSVERYLAICH (SEQ ID NO:43); Portion of human NTS1R TATVKVVIQVNTFMSFIFPMVVISV (SEQ ID NO:44); Portion of human δOR FTSIFTLTMMSVDRYIAVCH (SEQ ID NO:45); Portion of human δOR SWYWDTVTKICVFLFAFVVPILIITV (SEQ ID NO:46); Portion of human κOR FTSIFTLTMMSVDRYIAVCH (SEQ ID NO:47); Portion of human κOR YSWWDLFMKICVFIFAFVIPVLIIIV (SEQ ID NO:48); Portion of human μOR FTSIFTLCTMSVDRYIAVCH (SEQ ID NO:49); Portion of human μOR TWYWENLLKICVFIFAFIMPVLIITV (SEQ ID NO:50); Portion of human PAR2 YCSILFMTCLSVQRYWVIVN (SEQ ID NO:51); Portion of human PAR2 LVGDMFNYFLSLAIGVFLFPAFLTAS (SEQ ID NO:52). (FIG. 10B-10Q) AS408 preferentially modulates agonist-stimulated β-arrestin 2 recruitment on β₂AR and β₁AR compared to other Family A GPCRs.

FIG. 11. Binding of allosteric modulators to the lipid-facing allosteric pocket formed between TM3 and TM5 in GPCRs. Structure of AS408 bound to β₂AR with respect to orthosteric ligand alprenolol in comparison to positive allosteric modulator (AP8) bound to free-fatty acid receptor 1 (FFAR1 or GPR40), in the presence of orthosteric partial agonist MK-8666 (PDB: 5TZR).

FIGS. 12A-12D. FIGS. 12A and 12B show that either a neutral water molecule or a hydronium cation can mediate this interaction between E122^{3.41} and V206^{5.45}. FIG. 12C shows the agonist-induced transition into the active state. FIG. 12D shows that the cationic side chain of E122R is expected to directly interact with the V206^{5.45} backbone oxygen stabilizing the inactive receptor conformation.

FIG. 13. The figure shows MD simulations of L122 mutant of β₂AR.

FIG. 14. The figure shows that TM4 moves towards TM3 in all simulations, eventually the result of missing crystal contacts, and movement of TM3 around S207 only present in L122 simulations. Carbonyl of S207 moves towards TM3. Amide connecting S207 and Phe208 loses H-bonds stabilizing α-helix. Carbonyl O of S207 forms H-bond to water in wild type.

FIG. 15. The figure shows crystal structure of AS408 bound to the alprenolol-bound beta2-adrenergic receptor.

FIGS. 16A-16C. The figures show the concentration dependence of AS408 on norepinephrine-stimulated G protein activation (see FIG. 16A), on adenylyl cyclase activation (see FIG. 16B), and on arrestin recruitment (see FIG. 16C) by the β₂AR receptor.

FIG. 17A-B. Pharmacological characterization of AS408 by radioligand binding analysis to β₂AR (wt) and E122^{3.41} mutants. Membranes prepared from Sf9 cells infected with baculoviruses expressing β₂AR (wt) or E122 mutants in the absence or presence of co-expressed Gs heterotrimer were assessed by radioligand binding with [³H]DHAP. Inhibition of [³H]DHAP binding by full agonists epinephrine and norepinephrine and inverse agonist ICI-118,551 was measure in the absence or presence of 30 μM AS408. Ki's were determined using Graphpad (Prism, San Diego) using the Kd of [³H]DHAP specific for β₂AR (wt) and each mutant, according to the Cheng-Prusoff equation. Values for high affinity agonist site (K_{high}) and low affinity site (K_{low}) from membranes prepared from β₂AR (wt) or mutants co-infected with Gs heterotrimer were determined by non-linear regression fitting (2-site) using Kd values of [³H]DHAP, as above (Graphpad, San Diego).

FIG. 18A-18F. Mutation of E122^{3.41} influences stability of hydrogen bond network, observed in molecular dynamics (MD) simulations. Figures (FIG. 18A-18C) illustrate the networks involving position 122^{3.41} at β₂AR wild type (E122^{3.41}) and putative interactions of the mutants Q122^{3.41} and R122^{3.41}. These networks include E, Q or R122^{3.41}, a mediating water, V206^{5.45} and S207^{5.46}. The R122^{3.41} mutant was modeled to directly interact with V206^{5.45} and S207^{5.46} excluding the water molecule found in the wild type crystal structure (PDB: 2RH1). The interactions to be analyzed are marked by “1”, 2” and “3”. (FIG. 18D-18 F) MD simulations. (FIG. 18D, 18E) The polar network stays intact for the simulations of wild type β₂AR (E122^{3.41}) and its mutant Q122^{3.41}. This includes a weaker interaction between the mediating water molecule and the backbone oxygen of Ser207^{5.46} for the Q122^{3.41} mutant, observable in higher rmsd levels for the water molecule and a less frequent interaction to the backbone oxygen of Ser207^{5.46} (97% for E122^{3.41}, 85% for Q122^{3.41}). Representative snapshots of the MD simulations of β₂AR wild type and the Q122^{3.41} mutant superimposed with the β₂AR crystal structure or the modeled Q122^{3.41} mutant are shown in blue and grey or red and grey, respectively. (FIG. 18F) R122^{3.41} does not maintain the full polar network, as its side chain rotates away from Ser207^{5.46} but maintains a stable interaction to V206^{5.45}. A representative snapshot of the R122^{3.41} mutant simulations superimposed with the modeled R122^{3.41} mutant is shown in green and grey, respectively.

FIG. 19A-19L. AS408 utilizes E122^{3.41} of β₂AR, a residue that participates in an allosteric network. NAM activity of AS408: (FIG. 19A, FIG. 19D, FIG. 19H) β₂AR (wt), (FIG. 19B, FIG. 19E, FIG. 19I) is diminished in E122Q (FIG. 19C, FIG. 19F, FIG. 19J) E122L, and (FIG. 19G, FIG. 19K) in E122R, in norepinephrine-stimulated β-arrestin 2 recruitment, (FIG. 19A-FIG. 19C), [³⁵S]GTPγS binding, (FIG. 19D-FIG. 19G), and cAMP accumulation, (FIG. 19H-FIG. 19K) compared to β₂AR (wt). (FIG. 19L) β₂AR (E122R) displayed a higher basal activity but was unresponsive to inverse agonist ICI-118,551.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂₋. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multi cyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbomenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multi cyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multi cyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

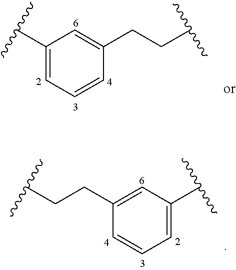 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR, ═O, ═NR′, ═N—OR′, —NR′R″, —SR, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R, —S(O)₂NR′R″, —NRSO₂R′, —NR′NR″R″′, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCB, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCH Cl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CHCl₂, —CHBr₂, —CHF₂, —CHI₂, —CH₂CI, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂CI, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₁₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₁₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroaryl ene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroaryl ene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroaryl ene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroaryl ene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkyl ene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkyl ene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on a R substituent or L linker or an “openly substituted” R substituent or L linker), the recited R substituent or L linker may, in embodiments, be substituted with one or more “first substituent group(s)” as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1} As a further example, L¹ may be substituted with one or more first substituent groups denoted by R^{L1.1} L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g. R^{1.1}, R^{2.1}, R^{3.1}, R^{4.1}, R^{5.1} . . . R^{100.2}; R^{1A.1}, R^{2A.1}, R^{3A.1}, R^{4A.1}, R^{5A.1}, R^{100A.1}; R^{L1.1}, R^{L2.1}, R^{L3.1}, R^{L4.1}, R^{L5.1}, R^{L100.1}) may be further substituted with one or more second substituent groups (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}. R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g. R^{1.2}, R^{2.2}, R^{3.2}, R^{4.2}, R^{5.2}, R^{100.2}; R^{1A.2}, R^{2A.2}, R^{3A.2}, R^{4A.2}, R^{5A.2} . . . R^{100A.2}. R^{L1.2}, R^{L2.2}, R^{L3.2}, R^{L4.2}, R^{L5.2} . . . R^{L100.2}) may be further substituted with one or more third substituent groups (e.g. R^{1.3}, R^{2.3}, R^{3.3}, R^{4.3}, R^{5.3}, R^{100.3}. R^{1A.3}, R^{2A.3}, R^{3A.3}, R^{4A.3}, R^{5A.3}, R^{100A.3}; R^{L1.3}, R^{L2.3}, R^{L3.3}, R^{L4.3}, R^{L5.3}, R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B. etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different.

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁-C₈, C₁-C₆, C₁-C₄, C₁-C₂ alkyl (e.g., saturated); C₂-C₈, C₂-C₆ or C₂-C₄ alkenyl or alkynyl), R^{WW.2}-substituted or unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), R^{WW.2}-substituted or unsubstituted cycloalkyl (e.g., C₃-C₈, C₃-C₆, C₄-C₆, or C₅-C₆ cycloalkyl (e.g., saturated) or cycloalkenyl), R^{WW.2}-substituted or unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{WW.3}-substituted or unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), R^{WW.3}-substituted or unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), R^{WW.3}-substituted or unsubstituted cycloalkyl (e.g., C₃-C₈, C₃-C₆, C₄-C₆, or C₅-C₆ cycloalkyl (e.g., saturated) or cycloalkenyl), R^{WW.3}-substituted or unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first ring substituent group is optionally different. Each second ring substituent group is optionally different. Each third ring substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.1}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —C N, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.2}-substituted or unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), R^{LWW.2}-substituted or unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), R^{LWW.2}-substituted or unsubstituted cycloalkyl (e.g., C₃-C₈, C₃-C₆, C₄-C₆, or C₅-C₆ cycloalkyl (e.g., saturated) or cycloalkenyl), R^{LWW.2}, substituted or unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), R^{LWW.2}-substituted or unsubstituted aryl (e.g., C₆-C₁₂, C₆-C₁₀, or phenyl), or R^{LWW.2}, substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{LWW.3}-substituted or unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), R^{LWW.3}-substituted or unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), R^{WW.3}-substituted or unsubstituted cycloalkyl (e.g., C₃-C₈, C₃-C₆, C₄-C₆, or C₅-C₆ cycloalkyl (e.g., saturated) or cycloalkenyl), R^{LWW.3}, substituted or unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂ alkyl(e.g., saturated); C₂-C₈, C₂-C₆ or C₂-C₄ alkenyl or alkynyl), unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), unsubstituted cycloalkyl (e.g., C₃-C₈, C₃-C₆, C₄-C₆, or C₅-C₆ cycloalkyl (e.g., saturated) or cycloalkenyl), unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3}, is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.1}-substituted or unsubstituted alkyl (e.g., C₁-C₈, C₁-C₆, C₁-C₄, or C₁-C₂ alkyl (e.g., saturated); C₂-C₈, C₂-C₆ or C₂-C₄ alkenyl or alkynyl), R^{WW.1}-substituted or unsubstituted heteroalkyl (e.g., 2 to 8 membered, 2 to 6 membered, 4 to 6 membered, 2 to 3 membered, or 4 to 5 membered heteroalkyl (e.g., saturated); 3 to 8 membered, 3 to 6 membered, 4 to 6 membered, or 4 to 5 membered heteroalkenyl or heteroalkynyl), R^{WW.1}-substituted or unsubstituted cycloalkyl (e.g., C₃-C₈, C₃—C₆, C₄-C₆, or C₅-C₆ cycloalkyl (e.g., saturated) or cycloalkenyl), R^{WW.1}-substituted or unsubstituted heterocycloalkyl (e.g., 3 to 8 membered, 3 to 6 membered, 4 to 6 membered, 4 to 5 membered, or 5 to 6 membered heterocycloalkyl (e.g., saturated) or heterocycloalkenyl), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). Again, “WW” represents the stated superscript number of the subject R group (e.g. 1, 2, 3, 1A, 2A, 3A, 1B, 2B, 3B. etc.). R^{WW.1}, as well as X^{WW}, R^{WW.2}, and R^{WW.3}, are as defined above.

In the event that any L linker group recited in a claim or chemical formula description set forth herein (i.e. an L^{WW} substituent) is not explicitly defined, then that L group (L^{WW} group) is herein defined as independently —O—, —NH—, —COO—, —CONH—, —S—, —SO₂NH—, R^{LWW.1}-substituted or unsubstituted alkylene (e.g., C₁-C₈, C₁-C₆, C₁-C₄, or C₁-C₂ alkylene (e.g., saturated); C₂-C₈, C₂-C₆ or C₂-C₄ alkenylene or alkynylene), R^{LWW.1}-substituted or unsubstituted heteroalkylene (e.g., 2 to 8 membered, 2 to 6 membered, 4 to 6 membered, 2 to 3 membered, or 4 to 5 membered heteroalkylene (e.g., saturated); 3 to 8 membered, 3 to 6 membered, 4 to 6 membered, or 4 to 5 membered heteroalkenylene or heteroalkynylene), R^{LWW.1}-substituted or unsubstituted cycloalkylene (e.g., C₃-C₈, C₃-C₆, C₄-C₆, or C₅-C₆ cycloalkylene (e.g., saturated) or cycloalkenylene), R^{LWW.1}-substituted or unsubstituted heterocycloalkylene (e.g., 3 to 8 membered, 3 to 6 membered, 4 to 6 membered, 4 to 5 membered, or 5 to 6 membered heterocycloalkylene (e.g., saturated) or heterocycloalkenylene), R^{LWW.1}-substituted or unsubstituted arylene (e.g., C₆-C₁₂, C₆-C₁₀, or phenylene), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B. etc.). R^{LWW.1} is as defined above.

For example, an R^{WW} substituent may be substituted with a first substituent group R^{WW.1}. When R^{WW} is phenyl, the said phenyl group is optionally substituted by one or more R^{WW.1}. When R^{WW.1} is substituted alkyl (e.g., methyl), the said alkyl group is optionally substituted by one or more R^{WW.2}. The compound that could be formed may include, but are not limited to, the compounds depicted below wherein R^{WW.2} is optionally substituted cyclopentyl, optionally substituted pyridyl, NH₂, or optionally substituted benzoxazolyl, wherein each such optionally substituted R^{WW.2} substituent group is optionally substituted with one or more R^{WW.3}. By way of non-limiting examples, such R^{WW.3} substituents could be independently unsubstituted alkyl (e.g., ethyl), halogen (e.g., fluoro), or OH, as shown below.

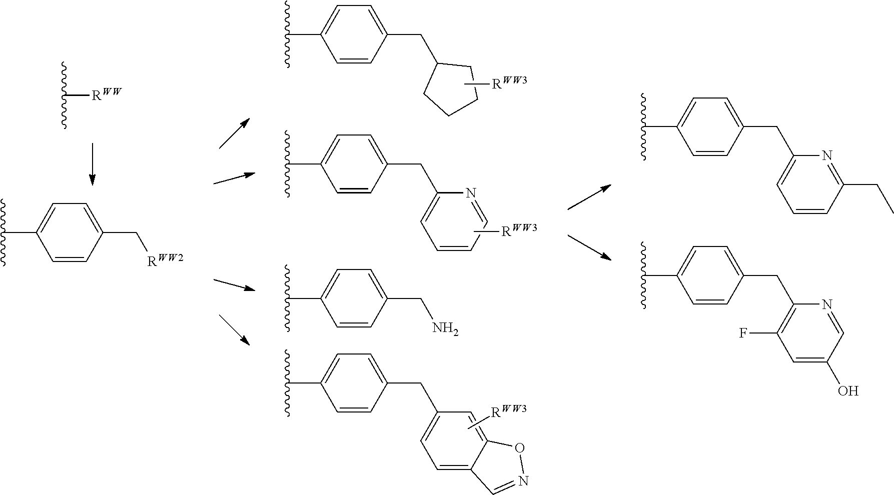

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₁₀ alkyl, or unsubstituted 2 to 20 membered heteroalkyl,” the group may contain one or more unsubstituted C₁-C₁₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R^{1.3} substituents are present, each R^{1.3} substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R^{1.3} and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CFb). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accordance with its well understood meaning and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety or a compound (e.g., as described herein) that is able to measurably bind to a target (e.g., β2 adrenergic receptor). In embodiments, where a moiety or compound is capable of binding a target, the moiety or compound is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g, norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

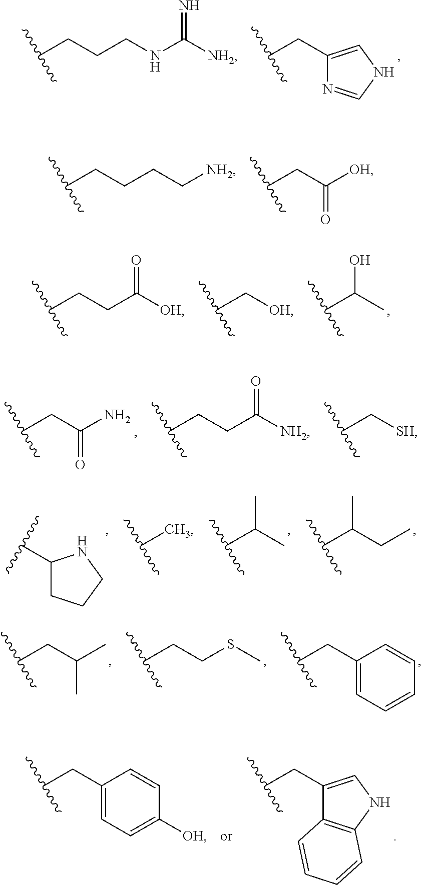

The term “non-natural amino acid side chain” refers to the functional substituent of compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride,cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. 1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge el al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

An “inhibitor” refers to a compound (e.g. compounds described herein) that reduces activity when compared to a control, such as absence of the compound or a compound with known inactivity. The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The terms “lung disease,” “pulmonary disease,” “pulmonary disorder,” etc. are used interchangeably herein. The term is used to broadly refer to lung disorders characterized by difficulty breathing, coughing, airway discomfort and inflammation, increased mucus, and/or pulmonary fibrosis. Examples of lung diseases include lung cancer, cystic fibrosis, asthma, Chronic Obstructive Pulmonary Disease (COPD), bronchitis, emphysema, bronchiectasis, pulmonary edema, pulmonary fibrosis, sarcoidosis, pulmonary hypertension, pneumonia, tuberculosis, Interstitial Pulmonary Fibrosis (IPF), Interstitial Lung Disease (ILD), Acute Interstitial Pneumonia (AIP), Respiratory Bronchiolitis-associated Interstitial Lung Disease (RBILD), Desquamative Interstitial Pneumonia (DIP), Non-Specific Interstitial Pneumonia (NSIP), Idiopathic Interstitial Pneumonia (IIP), Bronchiolitis obliterans, with Organizing Pneumonia (BOOP), restrictive lung disease, or pleurisy.

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

As used herein, the term “cardiovascular disorder” or “cardiovascular disease” is used in accordance with its plain ordinary meaning. In embodiments, cardiovascular diseases that may be treated with a compound, pharmaceutical composition, or method described herein include, but are not limited to, stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing (i.e., the patient or subject to be treated has the disease to be treated).

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein may include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “β₂AR receptor” or “β₂AR” or “β₂ adrenoreceptor” or “ADRB2” refers to the protein “beta-2 adrenergic receptor”. In embodiments, “β₂AR receptor” or “β₂AR” or “β₂ adrenoreceptor” or “ADRB2” refers to the human protein. Included in the term “β₂AR receptor” or “β₂AR” or “β₂ adrenoreceptor” or “ADRB2” are the wildtype and mutant forms of the protein. In embodiments, “β₂AR receptor” or “β₂AR” or “β₂ adrenoreceptor” or “ADRB2” refers to the protein associated with Entrez Gene 154, UniProt P07550, and/or RefSeq (protein) NP 000015. In embodiments, the reference numbers immediately above refer to the protein, and associated nucleic acids, known as of the date of filing of this application. In embodiments, “β₂AR receptor” or “β₂AR” or “β₂ adrenoreceptor” or “ADRB2” refers to the wildtype human protein. In embodiments, “β₂AR receptor” or “β₂AR” or “β₂ adrenoreceptor” or “ADRB2” refers to the wildtype human nucleic acid. In embodiments, the β₂AR receptor is a mutant β₂AR receptor. In embodiments, the mutant β₂AR receptor is associated with a disease that is not associated with wildtype β₂AR receptor. In embodiments, the β₂AR receptor includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype β₂AR receptor. In embodiments, the β₂AR receptor has the protein sequence corresponding to RefSeq NP_000015.1. In embodiments, the β₂AR receptor has the protein sequence corresponding to RefSeq NM_000024.5. In embodiments, the β₂AR receptor has the following amino acid sequence:

(SEQ ID NO: 53)
MGQPGNGSAFLLAPNRSHAPDHDVTQQRDEVWVVGMGIVMSLIVL
AIVFGNVLVITAIAKFERLQTVTNYFITSLACADLVMGLAVVPFG
AAHILMKMWTFGNFWCEFWTSIDVLCVTASIETLCVIAVDRYFAI
TSPFKYQSLLTKNKARVIILMVWIVSGLTSFLPIQMHWYRATHQE
AINCYANETCCDFFTNQAYAIASSIVSFYVPLVIMVFVYSRVFQE
AKRQLQKIDKSEGRFHVQNLSQVEQDGRTGHGLRRSSKFCLKEHK
ALKTLGIIMGTFTLCWLPFFIVNIVHVIQDNLIRKEVYILLNWIG
YVNSGFNPLIYCRSPDFRIAFQELLCLRRSSLKAYGNGYSSNGNT
GEQSGYHVEQEKENKLLCEDLPGTEDFVGHQGTVPSDNIDSQGRN
CSTNDSLL.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compounds

In an aspect is provided a compound having the formula:

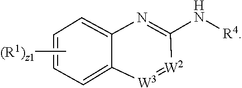

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); two R¹ substituents may optionally be joined to form a substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl).

z1 is an integer from 0 to 4.

W² is N, CH, or C(R²).

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl).

W³ is N, CH, or C(R³).

R³ is independently halogen, —CX³₃, —CHX³₂, —CH₂X³, —OCX³₃, —OCH₂X³, —OCHX³₂, —CN, —SOₙ₁R³⁰, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl).

R⁴ is independently substituted (e.g., substituted with one or more substituent groups, size-limited substituents, and/or lower substituents) or unsubstituted aryl (e.g., C₆-C₁₀ aryl, C₁₀ aryl, or phenyl), substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted spirocycloalkyl, substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), hydrogen, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), or substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl).

R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); R^{1A} and R^{1B} substituents bonded to the same nitrogen atom may optionally be joined to form a substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); R^{2A} and R^{2B} substituents bonded to the same nitrogen atom may optionally be joined to form a substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); and R^{3A} and R^{3B} substituents bonded to the same nitrogen atom may optionally be joined to form a substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl).

X, X¹, X², and X³ are independently —F, —Cl, —Br, or —I.

n1, n2, and n3 are independently an integer from 0 to 4.

m1, m2, m3, v1, v2, and v3 are independently 1 or 2.

In embodiments, R⁴ is independently substituted (e.g., substituted with one or more substituent groups, size-limited substituents, and/or lower substituents) or unsubstituted aryl (e.g., C₆-C₁₀ aryl, C₁₀ aryl, or phenyl), substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted spirocycloalkyl, substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), hydrogen, or substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl).

In embodiments, R⁴ is substituted or unsubstituted phenyl, substituted or unsubstituted naphthyl, substituted or unsubstituted pyridinyl or substituted or unsubstituted pyrimidinyl.

In embodiments, R⁴ is substituted (e.g., substituted with one or more substituent groups, size-limited substituents, and/or lower substituents) or unsubstituted phenyl. In embodiments, R⁴ is substituted or unsubstituted naphthyl. In embodiments, R⁴ is substituted or unsubstituted pyridinyl. In embodiments, R⁴ is substituted or unsubstituted pyrimidinyl. In embodiments, R⁴ is substituted phenyl. In embodiments, R⁴ is unsubstituted phenyl. In embodiments, R⁴ is substituted naphthyl. In embodiments, R⁴ is unsubstituted naphthyl. In embodiments, R⁴ is substituted pyridinyl. In embodiments, R⁴ is unsubstituted pyridinyl. In embodiments, R⁴ is substituted pyrimidinyl. In embodiments, R⁴ is unsubstituted pyrimidinyl.

In embodiments, R⁴ is (substituted alkyl)-substituted phenyl. In embodiments, R⁴ is (substituted alkoxy)-substituted phenyl. In embodiments, R⁴ is (substituted heteroalkyl)-substituted phenyl. In embodiments, R⁴ is (substituted C₁-C₄ alkyl)-substituted phenyl. In embodiments, R⁴ is (substituted 2 to 5 membered heteroalkyl)-substituted phenyl. In embodiments, R⁴ is (substituted alkyl)-substituted phenyl. In embodiments, R⁴ is (unsubstituted alkoxy)-substituted phenyl. In embodiments, R⁴ is (unsubstituted heteroalkyl)-substituted phenyl. In embodiments, R⁴ is (unsubstituted C₁-C₄ alkyl)-substituted phenyl. In embodiments, R⁴ is (unsubstituted 2 to 5 membered heteroalkyl)-substituted phenyl. In embodiments, R⁴ is hydroxy substituted phenyl. In embodiments, R⁴ is halo substituted phenyl. In embodiments, R⁴ is —CH₂OH substituted phenyl. In embodiments, R⁴ is —CH₂CH₂COOH substituted phenyl. In embodiments, R⁴ is —CH₂CH₂COOCH₂CH(OH)CH₂OH substituted phenyl. In embodiments, R⁴ is —SO₂NH₂ substituted phenyl. In embodiments, R⁴ is —C(O)NHCH₃ substituted phenyl. In embodiments, R⁴ is —C(O)CH₃, substituted phenyl. In embodiments, R⁴ is —C(O)OCH₃ substituted phenyl.

In embodiments, the compound has the formula:

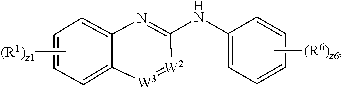

wherein R⁶ is independently halogen, —CX⁶₃, —CHX⁶₂, —CH₂X⁶, —OCX⁶₃, —OCH₂X⁶, —OCHX⁶₂, —CN, —SOₙ₃R^{6D}, —SOᵥ₃NR^{6A}R^{6B}, —NHC(O)NR^{6A}R^{6B}, —N(O)ₘ₃, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, —N₃, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₈ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); z6 is an integer from 0 to 5; R^{6A}, R^{6B}, R^{6C}, and R^{6D} are independently hydrogen, —CX₃, —CN, —COOH, —CONH₂, —CHX₂, —CH₂X, substituted (e.g., substituted with one or more substituent groups, size-limited substituents, and/or lower substituents) or unsubstituted alkyl (e.g., C₁-C₈ alkyl, C₁-C₆ alkyl, or C₁-C₄ alkyl), substituted (e.g., substituted with one or more substituent groups, size-limited substituents, and/or lower substituents) or unsubstituted heteroalkyl (e.g., 2 to 8 membered heteroalkyl, 2 to 6 membered heteroalkyl, or 2 to 4 membered heteroalkyl), substituted (e.g., substituted with one or more substituent groups, size-limited substituents, and/or lower substituents) or unsubstituted cycloalkyl (e.g., C₃-C₅ cycloalkyl, C₃-C₆ cycloalkyl, or C₅-C₆ cycloalkyl), substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with one or more substituent groups, size-limited substituents, and/or lower substituents) or unsubstituted aryl (e.g., C₆-C₁₀ aryl, C₁₀ aryl, or phenyl), or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); R^{6A} and R^{6B} substituents bonded to the same nitrogen atom may optionally be joined to form a substituted (e.g., substituted with one or more substituent groups, size-limited substituents, and/or lower substituents) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with one or more substituent groups, size-limited substituents, and/or lower substituents) or unsubstituted heteroaryl (e.g., 5 to 10 membered heteroaryl, 5 to 9 membered heteroaryl, or 5 to 6 membered heteroaryl); X⁶ is independently —F, —Cl, —Br, or —I; n6 is independently an integer from 0 to 4; and m6 and v6 are independently 1 or 2.

In embodiments, the compound has the formula:

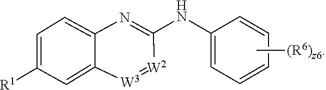

In embodiments, the compound has the formula:

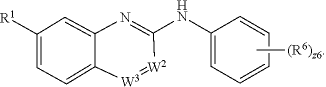

In embodiments, the compound has the formula:

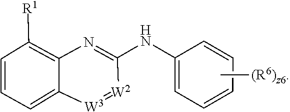

In embodiments, the compound has the formula:

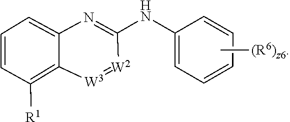

In embodiments, the compound has the formula:

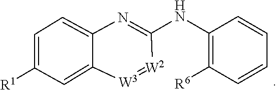

In embodiments, the compound has the formula:

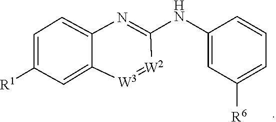

In embodiments, the compound has the formula:

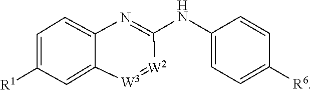

In embodiments, W² is N. In embodiments, W² is CH. In embodiments, W² is C(R²).

In embodiments, R² is halogen. In embodiments, R² is —NR^{2A}R^{2B}. In embodiments, R² is —NH₂. In embodiments, R² is —C(O)R^{2C}, —C(O)—OR^{2C}, or —C(O)NR^{2A}R^{2B}. In embodiments, R² is —COOH. In embodiments, R² is substituted or unsubstituted alkyl. In embodiments, R² is substituted or unsubstituted heteroalkyl. In embodiments, R² is unsubstituted alkyl. In embodiments, R² is methyl. In embodiments, R² is unsubstituted heteroalkyl.

In embodiments, W³ is C(R³). In embodiments, W³ is N. In embodiments, W³ is CH.

In embodiments, R³ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂CI, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

In embodiments, R³ is halogen. In embodiments, R³ is —NR^{3A}R^{3B}. In embodiments, R³ is —C(O)R^{3C}, —C(O)—OR^{3C}, or —C(O)NR^{3A}R^{3B}. In embodiments, R³ is substituted (e.g., substituted with one or more substituent groups, size-limited substituents, and/or lower substituents) or unsubstituted alkyl. In embodiments, R³ is unsubstituted alkyl. In embodiments, R³ is substituted (e.g., substituted with one or more substituent groups, size-limited substituents, and/or lower substituents) or unsubstituted heteroalkyl. In embodiments, R³ is unsubstituted heteroalkyl.

In embodiments, R³ is independently —NH₂, —OH, —O-alkyl (e.g., substituted (e.g., substituted with one or more substituent groups, size-limited substituents, and/or lower substituents) or unsubstituted —O—(C₁-C₈ alkyl), —O—(C₁-C₆ alkyl), —O—(C₁-C₄ alkyl), or —O—(C₁-C₂ alkyl)), —NH-alkyl (e.g., substituted (e.g., substituted with one or more substituent groups, size-limited substituents, and/or lower substituents) or unsubstituted —NH—(C₁-C₈ alkyl), —NH—(C₁-C₆ alkyl), —NH—(C₁-C₄ alkyl), or —NH—(C₁-C₂ alkyl)), —Md-cycloalkyl (e.g., substituted (e.g., substituted with one or more substituent groups, size-limited substituents, and/or lower substituents) or unsubstituted —NH—(C₃-C₈ cycloalkyl), —NH—(C₃-C₆ cycloalkyl), —NH—(C₄-C₆ cycloalkyl), or —NH—(C₅-C₆ cycloalkyl)), —N-dialkyl (i.e., —N(alkyl)₂, wherein the two alkyl groups are optionally different) (e.g., substituted (e.g., substituted with one or more substituent groups, size-limited substituents, and/or lower substituents) or unsubstituted —N—(C₁-C₈ alkyl)₂, —N—(C₁-C₆ alkyl)₂, —N—(C₁-C₄ alkyl)₂, or —N—(C₁-C₂ alkyl)₂), unsubstituted C₁-C₄ alkyl (e.g., C₁-C₃ alkyl, C₂-C₃ alkyl, or C₁-C₂ alkyl), —CN, —CF₃, —NO₂, —COOH, or —NHC(═NH)NH₂. In embodiments, R³ is —OH. In embodiments, R³ is —O-alkyl (e.g., substituted (e.g., substituted with one or more substituent groups, size-limited substituents, and/or lower substituents) or unsubstituted —O—(C₁-C₈ alkyl), —O—(C₁-C₆ alkyl), —O—(C₁-C₄ alkyl), or —O—(C₁-C₂ alkyl)). In embodiments, R³ is —NH-alkyl (e.g., substituted (e.g., substituted with one or more substituent groups, size-limited substituents, and/or lower substituents) or unsubstituted —NH—(C₁-C₈ alkyl), —NH—(C₁-C₆ alkyl), —NH—(C₁-C₄ alkyl), or —NH—(C₁-C₂ alkyl)). In embodiments, R³ is —NH-dialkyl (e.g., substituted (e.g., substituted with one or more substituent groups, size-limited substituents, and/or lower substituents) or unsubstituted —N—(C₁-C₈ alkyl)₂, —N—(C₁-C₆ alkyl)₂, —N—(C₁-C₄ alkyl)₂, or —N—(C₁-C₂ alkyl)₂). In embodiments, R³ is —COOH.

In embodiments, R³ is substituted (e.g., substituted with one or more substituent groups, size-limited substituents, and/or lower substituents) C₁-C₄ alkyl. In embodiments, R³ is substituted (e.g., substituted with one or more substituent groups, size-limited substituents, and/or lower substituents) methyl. In embodiments, R³ is substituted (e.g., substituted with one or more substituent groups, size-limited substituents, and/or lower substituents) ethyl. In embodiments, R³ is substituted (e.g., substituted with one or more substituent groups, size-limited substituents, and/or lower substituents) n-propyl. In embodiments, R³ is substituted (e.g., substituted with one or more substituent groups, size-limited substituents, and/or lower substituents) isopropyl. In embodiments, R³ is substituted (e.g., substituted with one or more substituent groups, size-limited substituents, and/or lower substituents) n-butyl. In embodiments, R³ is substituted (e.g., substituted with one or more substituent groups, size-limited substituents, and/or lower substituents) t-butyl. In embodiments, R³ is substituted (e.g., substituted with one or more substituent groups, size-limited substituents, and/or lower substituents) isobutyl. In embodiments, R³ is unsubstituted C₁-C₄ alkyl. In embodiments, R³ is unsubstituted methyl. In embodiments, R³ is unsubstituted ethyl. In embodiments, R³ is unsubstituted n-propyl. In embodiments, R³ is unsubstituted isopropyl. In embodiments, R³ is unsubstituted n-butyl. In embodiments, R³ is unsubstituted t-butyl. In embodiments, R³ is unsubstituted isobutyl.

In embodiments, R³ is —NH₂.

In embodiments, z1 is 0. In embodiments, z1 is 1. In embodiments, z1 is 2. In embodiments, z1 is 3. In embodiments, z1 is 4.

In embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

In embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, unsubstituted C₁-C₄ alkyl, unsubstituted phenyl, or unsubstituted 5 to 6 membered heteroaryl. In embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, or unsubstituted C₁-C₄ alkyl.

In embodiments, R¹ is independently halogen, —CF₃, unsubstituted C₁-C₄ alkyl, or unsubstituted phenyl. In embodiments, R¹ is independently halogen, —CF₃, or unsubstituted C₁-C₄ alkyl.

In embodiments, R¹ is independently halogen or —CF₃.

In embodiments, R¹ is independently —Cl, —Br, —I, or —CF₃.

In embodiments, R¹ is independently-Cl. In embodiments, R¹ is independently-Br. In embodiments, R¹ is independently —I. In embodiments, R¹ is independently —F.

In embodiments, R¹ is independently —CF₃. In embodiments, R¹ is independently —CBr₃. In embodiments, R¹ is independently —CCl₃. In embodiments, R¹ is independently —CI₃. In embodiments, R¹ is independently —CHF₂. In embodiments, R¹ is independently —CHBr₂. In embodiments, R¹ is independently —CHCl₂. In embodiments, R¹ is independently —CHI₂. In embodiments, R¹ is independently —CH₂F. In embodiments, R¹ is independently —CH₂Br. In embodiments, R¹ is independently —CH₂C₁. In embodiments, R¹ is independently —CH₂I.

In embodiments, R¹ is independently substituted (e.g., substituted with one or more substituent groups, size-limited substituents, and/or lower substituents) C₁-C₄ alkyl. In embodiments, R¹ is independently substituted (e.g., substituted with one or more substituent groups, size-limited substituents, and/or lower substituents) methyl. In embodiments, R¹ is independently substituted (e.g., substituted with one or more substituent groups, size-limited substituents, and/or lower substituents) ethyl. In embodiments, R¹ is s independently substituted (e.g., substituted with one or more substituent groups, size-limited substituents, and/or lower substituents) n-propyl. In embodiments, R¹ is independently substituted (e.g., substituted with one or more substituent groups, size-limited substituents, and/or lower substituents) isopropyl. In embodiments, R¹ is independently substituted (e.g., substituted with one or more substituent groups, size-limited substituents, and/or lower substituents) n-butyl. In embodiments, R¹ is independently substituted (e.g., substituted with one or more substituent groups, size-limited substituents, and/or lower substituents) t-butyl. In embodiments, R¹ is independently substituted (e.g., substituted with one or more substituent groups, size-limited substituents, and/or lower substituents) isobutyl. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted n-propyl. In embodiments, R¹ is independently unsubstituted isopropyl. In embodiments, R¹ is independently unsubstituted n-butyl. In embodiments, R¹ is independently unsubstituted t-butyl. In embodiments, R¹ is independently unsubstituted isobutyl.

In embodiments, R¹ is independently unsubstituted phenyl. In embodiments, R¹ is independently unsubstituted 5 to 6 membered heteroaryl. In embodiments, R¹ is independently unsubstituted 5 membered heteroaryl. In embodiments, R¹ is independently unsubstituted 6 membered heteroaryl. In embodiments, R¹ is independently unsubstituted pyridyl. In embodiments, R¹ is independently unsubstituted pyrimidinyl. In embodiments, R¹ is independently unsubstituted furanyl. In embodiments, R¹ is independently unsubstituted thiophenyl. In embodiments, R¹ is independently unsubstituted pyrrolyl. In embodiments, R¹ is independently unsubstituted thiazolyl. In embodiments, R¹ is independently unsubstituted oxazolyl. In embodiments, R¹ is independently unsubstituted imidazolyl. In embodiments, R¹ is unsubstituted cyclopropyl. In embodiments, R¹ is unsubstituted cyclobutyl.

In embodiments, R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X. In embodiments, R^{1A}, R^{1B}, R^{1C}, R^{1D}, R^{2A}, R^{2B}, R^{2C}, R^{2D}, R^{3A}, R^{3B}, R^{3C}, and R^{3D} are independently substituted (e.g., substituted with one or more substituent groups, size-limited substituents, and/or lower substituents) alkyl or substituted (e.g., substituted with one or more substituent groups, size-limited substituents, and/or lower substituents) heteroalkyl. In embodiments, R^{1A}, R^{1B}, R^{1C}, R^{1D}, R^{2A}, R^{2B}, R^{2C}, R^{2D}, R^{3A}, R^{3B}, R^{3C}, and R^{3D} are independently unsubstituted alkyl or unsubstituted heteroalkyl. In embodiments R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen. In embodiments R^{1A} is independently hydrogen. In embodiments R^{1B} is independently hydrogen. In embodiments R^{1C} is independently hydrogen. In embodiments R^{1D} is independently hydrogen. In embodiments R^{2A} is independently hydrogen. In embodiments R^{2B} is independently hydrogen. In embodiments R^{2C} is independently hydrogen. In embodiments R^{2D} is independently hydrogen. In embodiments R^{3A} is independently hydrogen. In embodiments R^{3B} is independently hydrogen. In embodiments R^{3C} is independently hydrogen. In embodiments R^{3D} is independently hydrogen. In embodiments R^{1A}, R^{1B}, R^{1C}, R^{1D}, R^{2A}, R^{2B}, R^{2C}, R^{2D}, R^{3A}, R^{3B}, R^{3C}, and R^{3D} are independently unsubstituted C₁-C₄ alkyl. In embodiments R^{1A} is independently unsubstituted C₁-C₄ alkyl. In embodiments R^{1B} is independently unsubstituted C₁-C₄ alkyl. In embodiments R^{1C} is independently unsubstituted C₁-C₄ alkyl. In embodiments R^{1D} is independently unsubstituted C₁-C₄ alkyl. In embodiments R^{2A} is independently unsubstituted C₁-C₄ alkyl. In embodiments R^{2B} is independently unsubstituted C₁-C₄ alkyl. In embodiments R^{2C} is independently unsubstituted C₁-C₄ alkyl. In embodiments R^{2D} is independently unsubstituted C₁-C₄ alkyl. In embodiments R^{3A} is independently unsubstituted C₁-C₄ alkyl. In embodiments R^{3B} is independently unsubstituted C₁-C₄ alkyl. In embodiments R^{3C} is independently unsubstituted C₁-C₄ alkyl. In embodiments R^{3D} is independently unsubstituted C₁-C₄ alkyl. In embodiments R^{1A}, R^{1B}, R^{1C}, R^{1D}, R^{2A}, R^{2B}, R^{2C}, R^{2D}, R^{3A}, R^{3B}, R^{3C}, and R^{3D} are independently unsubstituted methyl. In embodiments R^{1A} is independently unsubstituted methyl. In embodiments R^{1B} is independently unsubstituted methyl. In embodiments R^{1C} is independently unsubstituted methyl. In embodiments R^{1D} is independently unsubstituted methyl. In embodiments R^{2A} is independently unsubstituted methyl. In embodiments R^{2B} is independently unsubstituted methyl. In embodiments R^{2C} is independently unsubstituted methyl. In embodiments R^{2D} is independently unsubstituted methyl. In embodiments R^{3A} is independently unsubstituted methyl. In embodiments R^{3B} is independently unsubstituted methyl. In embodiments R^{3C} is independently unsubstituted methyl. In embodiments R^{3D} is independently unsubstituted methyl.

In embodiments, X is independently —F, —Cl, —Br, or —I. In embodiments, X is independently —F. In embodiments, X is independently —Cl. In embodiments, X is independently —Br. In embodiments, X is independently —I. In embodiments, X¹ is independently —F, —Cl, —Br, or —I. In embodiments, X¹ is independently —F. In embodiments, X¹ is independently —Cl. In embodiments, X¹ is independently —Br. In embodiments, X¹ is independently —I. In embodiments, X² is independently —F, —Cl, —Br, or —I. In embodiments, X² is independently —F. In embodiments, X² is independently —Cl. In embodiments, X² is independently —Br. In embodiments, X² is independently —I. In embodiments, X³ is independently —F, —Cl, —Br, or —I. In embodiments, X³ is independently —F. In embodiments, X³ is independently —Cl. In embodiments, X³ is independently —Br. In embodiments, X³ is independently —I.

In embodiments, n1 is independently 0. In embodiments, n1 is independently 1. In embodiments, n1 is independently 2. In embodiments, n1 is independently 3. In embodiments, n1 is independently 4. In embodiments, n2 is independently 0. In embodiments, n2 is independently 1. In embodiments, n2 is independently 2. In embodiments, n2 is independently 3. In embodiments, n2 is independently 4. In embodiments, n3 is independently 0. In embodiments, n3 is independently 1. In embodiments, n3 is independently 2. In embodiments, n3 is independently 3. In embodiments, n3 is independently 4.

In embodiments, m1 is independently 1. In embodiments, m1 is independently 2. In embodiments, m2 is independently 1. In embodiments, m2 is independently 2. In embodiments, m3 is independently 1. In embodiments, m3 is independently 2. In embodiments, v1 is independently 1. In embodiments, v1 is independently 2. In embodiments, v2 is independently 1. In embodiments, v2 is independently 2. In embodiments, v3 is independently 1. In embodiments, v3 is independently 2.

In embodiments, R⁶ is independently halogen, —CX⁶₃, —CHX⁶₂, —CH₂X⁶, —OCX⁶₃, —OCH₂X⁶, —OCHX⁶₂, —CN, —SOₙ₃R⁶⁰, —SOᵥ₃NR^{6A}R^{6B}, —NHC(O)NR^{6A}R^{6B}, —N(O)ₘ₃, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, —N₃, substituted (e.g., substituted with one or more substituent groups, size-limited substituents, and/or lower substituents) or unsubstituted alkyl, substituted (e.g., substituted with one or more substituent groups, size-limited substituents, and/or lower substituents) or unsubstituted heteroalkyl, substituted (e.g., substituted with one or more substituent groups, size-limited substituents, and/or lower substituents) or unsubstituted cycloalkyl, substituted (e.g., substituted with one or more substituent groups, size-limited substituents, and/or lower substituents) or unsubstituted heterocycloalkyl, substituted (e.g., substituted with one or more substituent groups, size-limited substituents, and/or lower substituents) or unsubstituted aryl, or substituted (e.g., substituted with one or more substituent groups, size-limited substituents, and/or lower substituents) or unsubstituted heteroaryl.

In embodiments, R⁶ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted (e.g., substituted with one or more substituent groups, size-limited substituents, and/or lower substituents) or unsubstituted C₁-C₁₀ alkyl, substituted (e.g., substituted with one or more substituent groups, size-limited substituents, and/or lower substituents) or unsubstituted 2 to 10 membered heteroalkyl, substituted (e.g., substituted with one or more substituent groups, size-limited substituents, and/or lower substituents) or unsubstituted C₅-C₆ cycloalkyl, substituted (e.g., substituted with one or more substituent groups, size-limited substituents, and/or lower substituents) or unsubstituted 5 to 6 membered heterocycloalkyl, substituted (e.g., substituted with one or more substituent groups, size-limited substituents, and/or lower substituents) or unsubstituted phenyl, or substituted (e.g., substituted with one or more substituent groups, size-limited substituents, and/or lower substituents) or unsubstituted 5 to 6 membered heteroaryl.

In embodiments, R⁶ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂. In embodiments, R⁶ is independently —F, —Cl, —Br, or —I.

In embodiments, R⁶ is independently substituted or unsubstituted C₁-C₁₀ alkyl, substituted or unsubstituted 2 to 10 membered heteroalkyl, substituted or unsubstituted C₅-C₆ cycloalkyl, substituted or unsubstituted 5 to 6 membered heterocycloalkyl. In embodiments, R⁶ is independently substituted or unsubstituted C₁-C₁₀ alkyl. In embodiments, R⁶ is independently substituted or unsubstituted 2 to 10 membered heteroalkyl. In embodiments, R⁶ is independently substituted or unsubstituted C₅-C₆ cycloalkyl, In embodiments, R⁶ is independently substituted or unsubstituted 5 to 6 membered heterocycloalkyl. In embodiments, R⁶ is independently substituted or unsubstituted C₁-C₈ alkyl. In embodiments, R⁶ is independently substituted or unsubstituted 2 to 8 membered heteroalkyl. In embodiments, R⁶ is independently substituted or unsubstituted C₁-C₅ alkyl. In embodiments, R⁶ is independently substituted or unsubstituted 2 to membered heteroalkyl. In embodiments, R⁶ is independently substituted or unsubstituted C₁-C₃ alkyl. In embodiments, R⁶ is independently substituted or unsubstituted 2 to 4 membered heteroalkyl.

In embodiments, R⁶ is independently —CH₂OH, —CH₂CH₂COOH, —CH₂CH₂COOCH₂CH(OH)CH₂OH, —SO₂NH₂, —C(O)NHCH₃, —C(O)CH₃, —C(O)OCH₃, or —OH.

In embodiments, z6 is 0. In embodiments, z6 is 1. In embodiments, z6 is 2. In embodiments, z6 is 3. In embodiments, z6 is 4. In embodiments, z6 is 5.

In embodiments, R^{6A}, R^{6B}, R^{6C}, and R^{6D} are independently hydrogen, —CX₃, —CN, —COOH, —CONH₂, —CHX₂, —CH₂X. In embodiments, R^{6A}, R^{6B}, R^{6C}, and R^{6D} are independently substituted (e.g., substituted with one or more substituent groups, size-limited substituents, and/or lower substituents) or unsubstituted alkyl or substituted (e.g., substituted with one or more substituent groups, size-limited substituents, and/or lower substituents) or unsubstituted heteroalkyl. In embodiments, R^{6A}, R^{6B}, R^{6C}, and R^{6D} are independently substituted (e.g., substituted with one or more substituent groups, size-limited substituents, and/or lower substituents) alkyl or substituted (e.g., substituted with one or more substituent groups, size-limited substituents, and/or lower substituents) heteroalkyl. In embodiments, R^{6A}, R^{6B}, R^{6C}, and R^{6D} are independently unsubstituted alkyl or unsubstituted heteroalkyl. In embodiments R^{6A} is hydrogen. In embodiments R^{6B} is hydrogen. In embodiments R^{6C} is hydrogen. In embodiments R^{6D} is hydrogen.

In embodiments, X⁶ is independently —F. In embodiments, X⁶ is independently —Cl. In embodiments, X⁶ is independently —Br. In embodiments, X⁶ is independently —I.

In embodiments, n6 is independently 0. In embodiments, n6 is independently 1. In embodiments, n6 is independently 2. In embodiments, n6 is independently 3. In embodiments, n6 is independently 4.

In embodiments, m6 is independently 1. In embodiments, m6 is independently 2. In embodiments, v6 is independently 1. In embodiments, v6 is independently 2.

In embodiments, the compound has the formula:

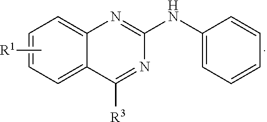

In embodiments, the compound has the formula:

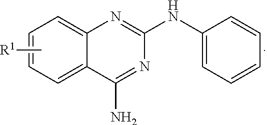

In embodiments, the compound has the formula:

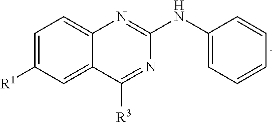

In embodiments, the compound has the formula:

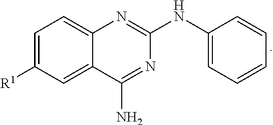

In embodiments, the compound has the formula:

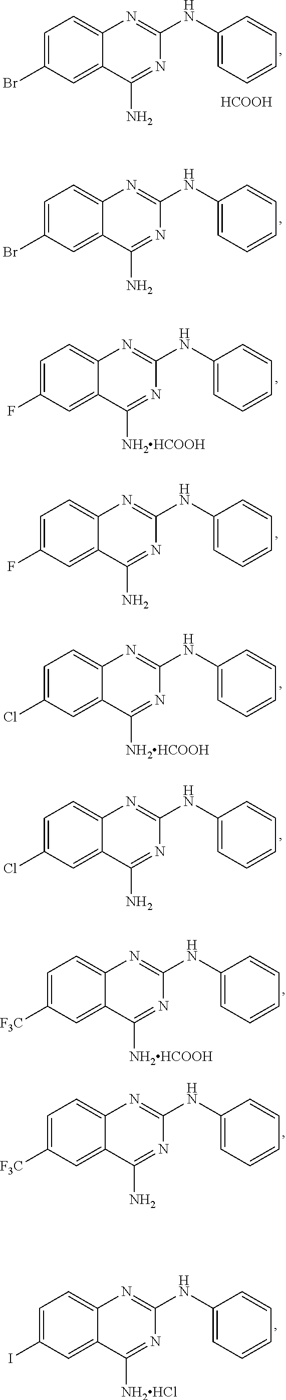

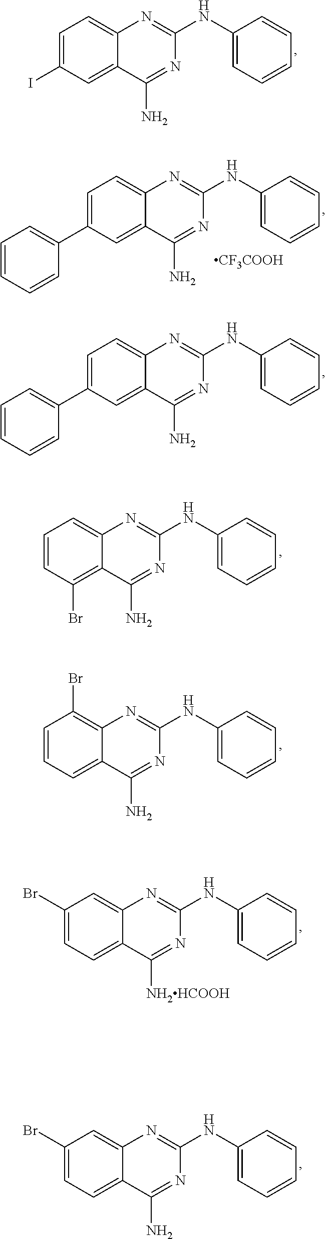

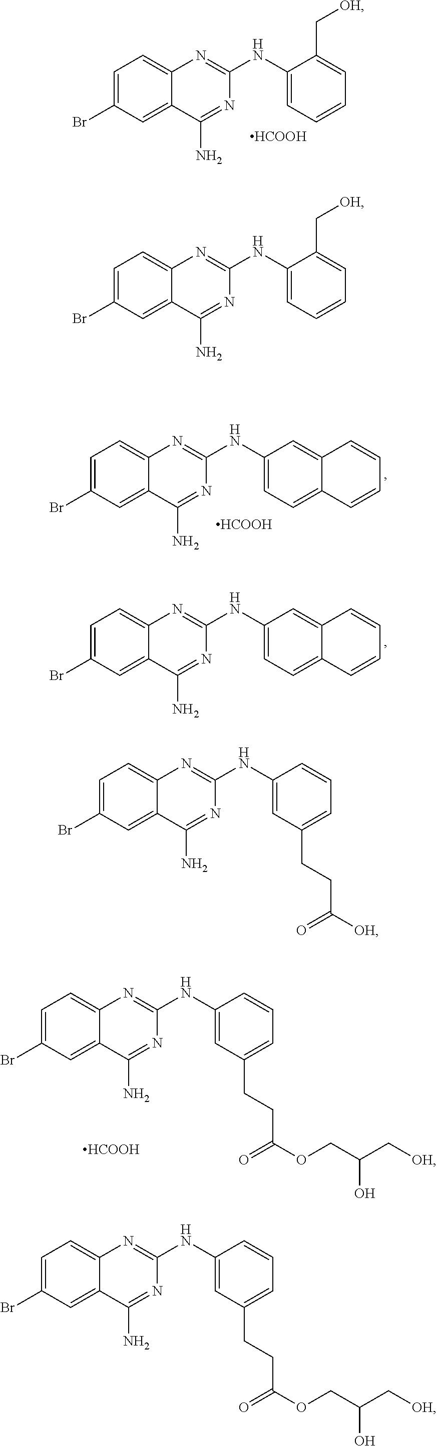

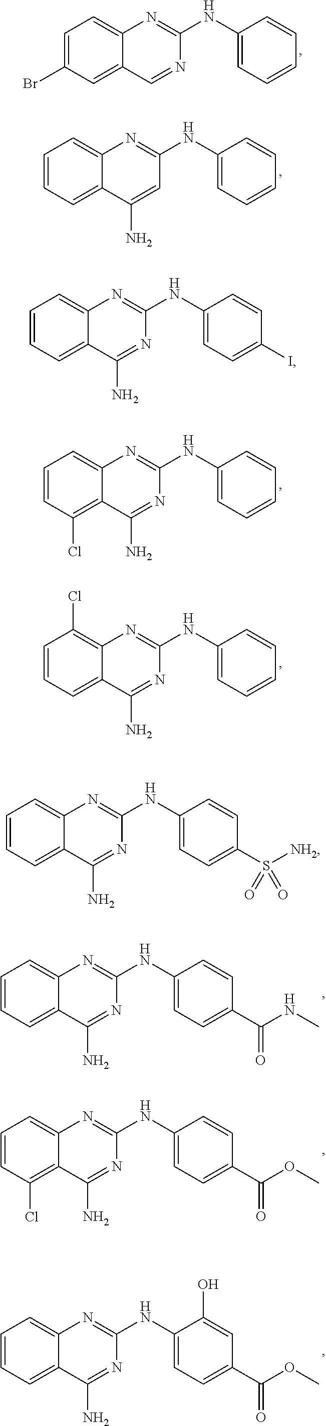

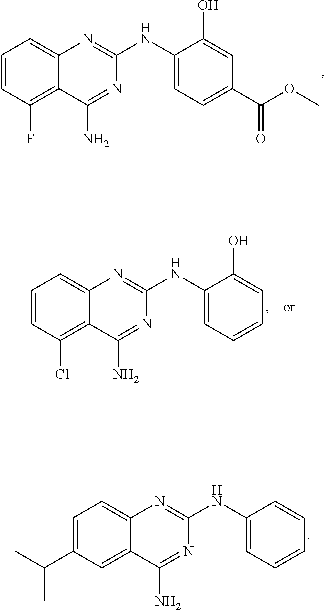

In embodiments, the compound has the formula:

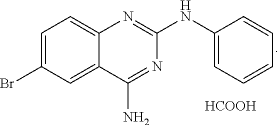 [SMILES: Nc1nc(Nc2ccccc2)nc2ccc(Br)cc12 ; "HCOOH"]

In embodiments, the compound has the formula:

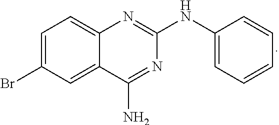 [SMILES: Nc1nc(Nc2ccccc2)nc2ccc(Br)cc12]

In embodiments, the compound has the formula:

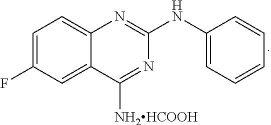

In embodiments, the compound has the formula:

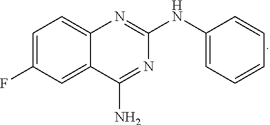 [SMILES: Nc1nc(Nc2ccccc2)nc2ccc(F)cc12]

In embodiments, the compound has the formula:

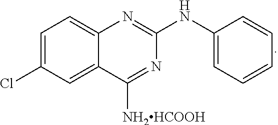

In embodiments, the compound has the formula:

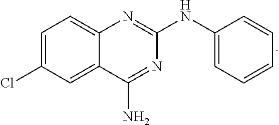 [SMILES: Nc1nc(Nc2ccccc2)nc2ccc(Cl)cc12]

In embodiments, the compound has the formula:

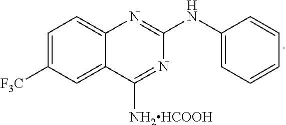

In embodiments, the compound has the formula:

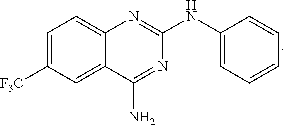 [SMILES: Nc1nc(Nc2ccccc2)nc2ccc(C(F)(F)F)cc12]

In embodiments, the compound has the formula:

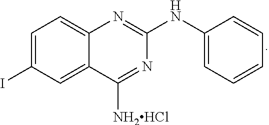

In embodiments, the compound has the formula:

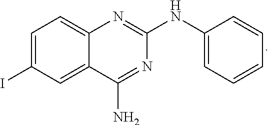 [SMILES: Nc1nc(Nc2ccccc2)nc2ccc(I)cc12]

In embodiments, the compound has the formula:

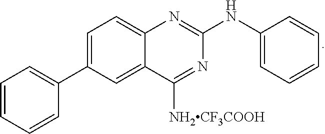

In embodiments, the compound has the formula:

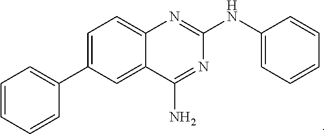 [SMILES: Nc1nc(Nc2ccccc2)nc2ccc(-c3ccccc3)cc12]

In embodiments, the compound has the formula:

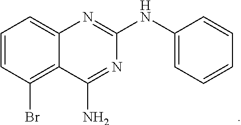 [SMILES: Nc1nc(Nc2ccccc2)nc2cccc(Br)c12]

In embodiments, the compound has the formula:

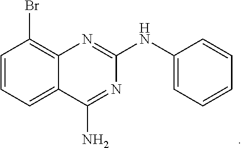 [SMILES: Nc1nc(Nc2ccccc2)nc2c(Br)cccc12]

In embodiments, the compound has the formula:

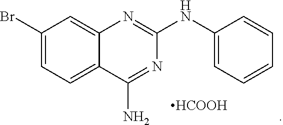 [SMILES: Nc1nc(Nc2ccccc2)nc2cc(Br)ccc12 ; "·HCOOH"]

In embodiments, the compound has the formula:

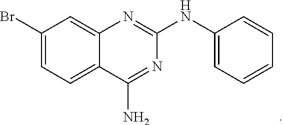 [SMILES: Nc1nc(Nc2ccccc2)nc2cc(Br)ccc12]

In embodiments, the compound has the formula:

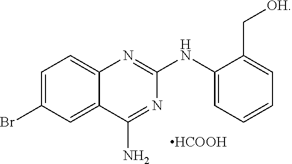 [SMILES: Nc1nc(Nc2ccccc2CO)nc2ccc(Br)cc12 ; "·HCOOH"]

In embodiments, the compound has the formula:

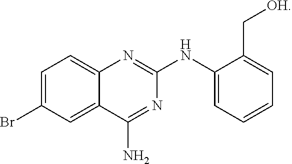 [SMILES: Nc1nc(Nc2ccccc2CO)nc2ccc(Br)cc12]

In embodiments, the compound has the formula:

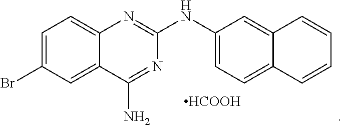 [SMILES: Nc1nc(Nc2ccc3ccccc3c2)nc2ccc(Br)cc12 ; "·HCOOH"]

In embodiments, the compound has the formula:

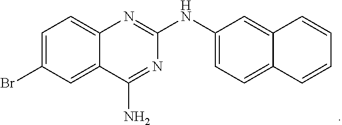 [SMILES: Nc1nc(Nc2ccc3ccccc3c2)nc2ccc(Br)cc12]

In embodiments, the compound has the formula:

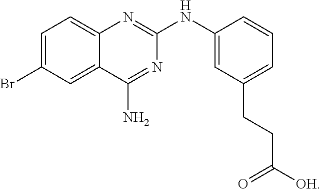 [SMILES: Nc1nc(Nc2cccc(CCC(=O)O)c2)nc2ccc(Br)cc12]

In embodiments, the compound has the formula:

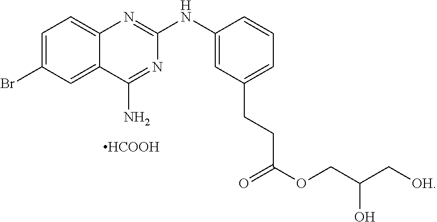 [SMILES: Nc1nc(Nc2cccc(CCC(=O)OCC(O)CO)c2)nc2ccc(Br)cc12 ; "·HCOOH"]

In embodiments, the compound has the formula:

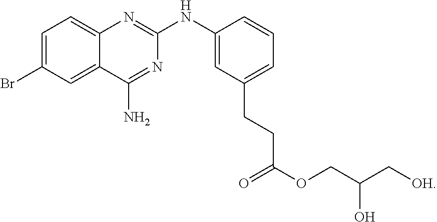 [SMILES: Nc1nc(Nc2cccc(CCC(=O)OCC(O)CO)c2)nc2ccc(Br)cc12]

In embodiments, the compound has the formula:

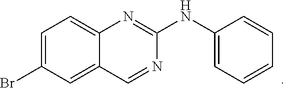 [SMILES: Brc1ccc2nc(Nc3ccccc3)ncc2c1]

In embodiments, the compound has the formula:

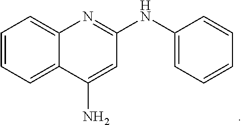 [SMILES: Nc1cc(Nc2ccccc2)nc2ccccc12]

In embodiments, the compound has the formula:

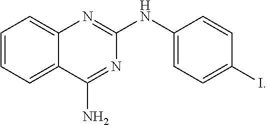 [SMILES: Nc1nc(Nc2ccc(I)cc2)nc2ccccc12]

In embodiments, the compound has the formula:

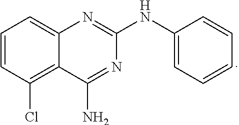 [SMILES: Nc1nc(Nc2ccccc2)nc2cccc(Cl)c12]

In embodiments, the compound has the formula:

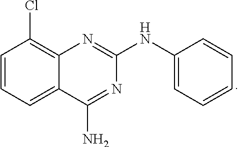 [SMILES: Nc1nc(Nc2ccccc2)nc2c(Cl)cccc12]

In embodiments, the compound has the formula:

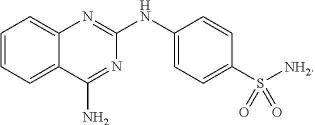 [SMILES: Nc1nc(Nc2ccc(S(N)(=O)=O)cc2)nc2ccccc12]

In embodiments, the compound has the formula:

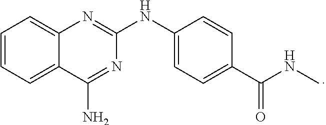 [SMILES: CNC(=O)c1ccc(Nc2nc(N)c3ccccc3n2)cc1]

In embodiments, the compound has the formula:

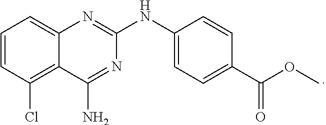 [SMILES: COC(=O)c1ccc(Nc2nc(N)c3c(Cl)cccc3n2)cc1]

In embodiments, the compound has the formula:

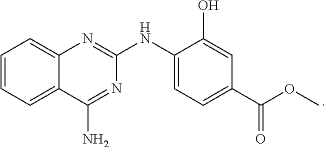 [SMILES: COC(=O)c1ccc(Nc2nc(N)c3ccccc3n2)c(O)c1]

In embodiments, the compound has the formula:

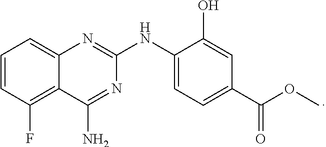 [SMILES: COC(=O)c1ccc(Nc2nc(N)c3c(F)cccc3n2)c(O)c1]

In embodiments, the compound has the formula:

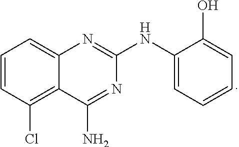 [SMILES: Nc1nc(Nc2ccccc2O)nc2cccc(Cl)c12]

In embodiments, the compound has the formula:

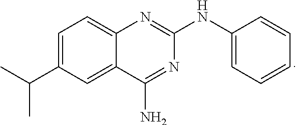 [SMILES: CC(C)c1ccc2nc(Nc3ccccc3)nc(N)c2c1]

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2A}, R^{2A.1}, R^{2A.2}, and R^{2A.3} have values corresponding to the values of R^{WW}, R™³, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3} respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B I}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6D}, R^{6D.1}, R^{6D.2}, and R^{6D.3}, respectively.

In embodiments, the compound (e.g. AS408) contacts an amino acid corresponding to C125^{3.44} of human β2 adrenergic receptor. In embodiments, the compound (e.g. AS408) contacts an amino acid corresponding to V126^{3.45} of human β2 adrenergic receptor. In embodiments, the compound (e.g. AS408) interacts with V129^{3.48} of human β2 adrenergic receptor. In embodiments, the compound (e.g. AS408) contacts an amino acid corresponding to V210^{5.49} of human β2 adrenergic receptor. In embodiments, the compound (e.g. AS408) contacts an amino acid corresponding to P211^{5.50} of human β2 adrenergic receptor. In embodiments, the compound (e.g. AS408) contacts an amino acid corresponding to I214^{5.53} of human β2 adrenergic receptor. In embodiments, the compound (e.g. AS408) contacts an amino acid corresponding to E122^{3.41} of human β2 adrenergic receptor. In embodiments, the primary amine of the compound (e.g. AS408) can hydrogen bond with an amino acid corresponding to E122^{3.41} of human β2 adrenergic receptor. In embodiments, the compound (e.g. AS408) contacts an amino acid corresponding to V206^{5.45} of human β2 adrenergic receptor. In embodiments, the compound (e.g. AS408) contacts an amino acid corresponding to the carbonyl of V206^{5.45} of human β2 adrenergic receptor. In embodiments, the primary amine of compound (e.g. AS408) contacts an amino acid corresponding to the carbonyl of V206^{5.45} of human β2 adrenergic receptor. In embodiments, the compound (e.g. AS408) contacts an amino acid corresponding to L45^{1.44} of human β2 adrenergic receptor. In embodiments, the bromine compound (e.g. AS408) contacts an amino acid corresponding to with L45^{1.44} of human β2 adrenergic receptor. In embodiments, the compound (e.g. AS408) contacts an amino acid corresponding to S207^{5.46} of human β2 adrenergic receptor.

In embodiments, the compound increases inhibition of β₂AR by an orthosteric antagonist (e.g., by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold). In embodiments, the compounds increases inhibition of β₂AR by an orthosteric inverse agonist (e.g., by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold). In embodiments, the compounds reduces activation of β₂AR by an orthosteric agonist (e.g., by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold).

In embodiments, the compound reduces binding of an orthosteric agonist to β₂AR (e.g., compared to a control such as absence of the compound). In embodiments, the compound increases binding of an orthosteric antagonist to β₂AR (e.g., compared to a control such as absence of the compound). In embodiments, the compound increases binding of an orthosteric inreverse agonist to β₂AR (e.g., compared to a control such as absence of the compound). In embodiments, the compound reduces β arrestin recruitment by β₂AR (e.g., compared to a control such as absence of the compound). In embodiments, the compound reduces cAMP accumulation (e.g., compared to a control such as absence of the compound). In embodiments, the compound reduces cAMP levels (e.g., compared to a control such as absence of the compound).

### III. Pharmaceutical Compositions

In an aspect is provided a pharmaceutical composition including a compound as disclosed herein, including embodiments, and a pharmaceutically acceptable excipient. In embodiments, compound is included in a therapeutically effective amount.

In an aspect, the pharmaceutical composition further includes a second agent, wherein the second agent is a β2 adrenergic receptor modulator. In embodiments, the second agent is a β2 adrenergic receptor inhibitor. In embodiments, the second agent is a β2 adrenergic receptor antagonist. In embodiments, the second agent is a β2 adrenergic receptor allosteric modulator. In embodiments, the second agent is a β2 adrenergic receptor allosteric inhibitor. In embodiments, the second agent is a β2 adrenergic receptor allosteric antagonist. In embodiments, the second agent is a β2 adrenergic receptor inverse agonist. In embodiments, the second agent is a β2 adrenergic receptor agonist.

In embodiments, the pharmaceutical composition further includes a second agent, wherein the second agent is a β2 adrenergic receptor inhibitor. In embodiments, the β2 adrenergic receptor inhibitor is butaxamine. In embodiments, the β2 adrenergic receptor inhibitor is butoxamine. In embodiments, the β2 adrenergic receptor inhibitor is ICI-118,551. In embodiments, the β2 adrenergic receptor inhibitor is propranolol. In embodiments, the second agent is included in a therapeutically effective amount. In embodiments, the second agent is an agent for treating a neurodegenerative disease. In embodiments, the second agent is an agent for treating Alzheimer's disease. In embodiments, the second agent is an agent for treating Amyotrophic lateral sclerosis. In embodiments, the second agent is an agent for treating Huntington's disease. In embodiments, the second agent is an agent for treating Parkinson's disease. In embodiments, the second agent is an agent for treating a pulmonary disease. In embodiments, the second agent is an agent for treating asthma. In embodiments, the second agent is an agent for treating a cardiovascular disease. In embodiments, the second agent is an agent for treating hypertension. In embodiments, the second agent is an agent for treating heart failure. In embodiments, the second agent is propranolol. In embodiments, the second agent is bucindolol. In embodiments, the second agent is carteolol. In embodiments, the second agent is carvedilol. In embodiments, the second agent is labetalol. In embodiments, the second agent is nadolol. In embodiments, the second agent is oxprenolol. In embodiments, the second agent is penbutolol. In embodiments, the second agent is pindolol. In embodiments, the second agent is sotalol. In embodiments, the second agent is timolol. In embodiments, the second agent inhibits β2 more than β1 (e.g. at least 1.5-, 2-, 3-, 4-, 5-, 6-, 7-, 8-, 9-, or 10-fold). In embodiments, the second agent inhibits β2 more than β1 (e.g. at least 10-, 15-, 20-, 25-, 30-, 35-, 40-, 45-, 50-, 60-, 70-, 80-, 90-, 100-fold). In embodiments, the second agent inhibits β2 more than β1 (e.g. at least 100-, 150-, 200-, 250-, 300-, 350-, 400-, 450-, 500-, 600-, 700-, 800-, 900-, or 1000-fold). In embodiments, the second agent inhibits β2 more than β1 (e.g. at least 1000-, 2000-, 3000-, 4000-, 5000-, 6000-, 7000-, 8000-, 9000-, or 10000-fold).

### IV. Methods of Use

In an aspect is provided a method of treating a disease associated with β2 adrenergic receptor, the method including administering to a subject in need thereof (e.g., a subject having the disease or a subject who may develop the disease) a therapeutically effective amount of a compound described herein, including embodiments.

In an aspect is provided a method of treating Parkinson's disease, hypertension, heart failure, asthma, myocardial infarction, angina pectoris, tachycardia, anxiety, tremor, migraine headache, cluster headache, hyperhidrosis, glaucoma, thyrotoxicosis, hyperthyroidism, esophageal variceal, ascites, post-traumatic stress disorder, psychogenic polydispsia, hemangioma, or cardiomyopathy, the method including administering to a subject in need thereof a therapeutically effective amount of a compound described herein, including embodiments.

In an aspect is provided a method of treating a neurodegenerative disease, the method including administering to a subject in need thereof (e.g., a subject having the neurodegenerative disease or a subject who may develop the neurodegenerative disease) a therapeutically effective amount of a compound described herein, including embodiments. In embodiments the neurodegenerative disease is Alzheimer's disease. In embodiments the neurodegenerative disease is Amyotrophic lateral sclerosis. In embodiments the neurodegenerative disease is Huntington's disease. In embodiments the neurodegenerative disease is Parkinson's disease.

In an aspect is provided a method of treating a pulmonary disease, the method including administering to a subject in need thereof (e.g., a subject having the pulmonary disease or a subject who may develop the pulmonary disease) a therapeutically effective amount of a compound described herein, including embodiments. In embodiments the pulmonary disease is asthma.

In an aspect is provided a method of treating a cardiovascular disease, the method including administering to a subject in need thereof (e.g., a subject having the cardiovascular disease or a subject who may develop the cardiovascular disease) a therapeutically effective amount of a compound described herein, including embodiments. In embodiments the cardiovascular disease in hypertension. In embodiments the cardiovascular disease is heart failure.

In embodiments, the method includes co-administering a second agent to the subject in need thereof, wherein the second agent is a β2 adrenergic receptor modulator (e.g., inhibitor, antagonist, inreverse agonist, agonist, allosteric modulator, allosteric inhibitor, or allosteric antagonist). In embodiments, the second agent is a β2 adrenergic receptor inhibitor. In embodiments, the second agent is a β2 adrenergic receptor antagonist. In embodiments, the second agent is a β2 adrenergic receptor allosteric modulator. In embodiments, the second agent is a β2 adrenergic receptor allosteric inhibitor. In embodiments, the second agent is a β2 adrenergic receptor allosteric antagonist. In embodiments, the second agent is a β2 adrenergic receptor inverse agonist. In embodiments, the second agent is a β2 adrenergic receptor agonist. In embodiments, the second agent is administered in a therapeutically effective amount.

In embodiments, the method includes administering a second agent to the subject in need thereof, wherein the second agent is a β2 adrenergic receptor modulator (e.g., inhibitor, antagonist, allosteric modulator, allosteric inhibitor, or allosteric antagonist). In embodiments, the second agent is a β2 adrenergic receptor inhibitor. In embodiments, the second agent is a β2 adrenergic receptor antagonist. In embodiments, the second agent is a β2 adrenergic receptor allosteric modulator. In embodiments, the second agent is a β2 adrenergic receptor allosteric inhibitor. In embodiments, the second agent is a β2 adrenergic receptor allosteric antagonist. In embodiments, the second agent is a β2 adrenergic receptor inverse agonist. In embodiments, the second agent is a β2 adrenergic receptor agonist. In embodiments, the second agent is an agent for treating a neurodegenerative disease. In embodiments, the second agent is an agent for treating Alzheimer's disease. In embodiments, the second agent is an agent for treating Amyotrophic lateral sclerosis. In embodiments, the second agent is an agent for treating Huntington's disease. In embodiments, the second agent is an agent for treating Parkinson's disease. In embodiments, the second agent is an agent for treating a pulmonary disease. In embodiments, the second agent is an agent for treating asthma. In embodiments, the second agent is an agent for treating a cardiovascular disease. In embodiments, the second agent is an agent for treating hypertension. In embodiments, the second agent is an agent for treating heart failure.

In an aspect is provided a method of treating a disease associated with β2 adrenergic receptor, the method including administering to a subject in need thereof (e.g., a subject having the disease or a subject who may develop the disease) a therapeutically effective amount of a compound described herein, including embodiments, and a β2 adrenergic receptor modulator (e.g., inhibitor, antagonist, inverse agonist, agonist, allosteric modulator, allosteric inhibitor, allosteric antagonist, orthosteric inhibitor, orthosteric antagonist, orthosteric inverse agonist, or orthosteric agonist).

In an aspect is provided a method of treating Parkinson's disease, hypertension, heart failure, asthma, myocardial infarction, angina pectoris, tachycardia, anxiety, tremor, migraine headache, cluster headache, hyperhidrosis, glaucoma, thyrotoxicosis, hyperthyroidism, esophageal variceal, ascites, post-traumatic stress disorder, psychogenic polydispsia, hemangioma, or cardiomyopathy, the method including administering to a subject in need thereof a therapeutically effective amount of a compound described herein, including embodiments, and a β2 adrenergic receptor modulator (e.g., inhibitor, antagonist, inverse agonist, agonist, allosteric modulator, allosteric inhibitor, allosteric antagonist, orthosteric inhibitor, orthosteric antagonist, orthosteric inverse agonist, or orthosteric agonist).

In an aspect is provided a method of treating a neurodegenerative disease, the method including administering to a subject in need thereof (e.g., a subject having the neurodegenerative disease or a subject who may develop the neurodegenerative disease) a therapeutically effective amount of a compound described herein, including embodiments, and a β2 adrenergic receptor modulator (e.g., inhibitor, antagonist, inverse agonist, agonist, allosteric modulator, allosteric inhibitor, allosteric antagonist, orthosteric inhibitor, orthosteric antagonist, orthosteric inverse agonist, or orthosteric agonist). In embodiments the neurodegenerative disease is Alzheimer's disease. In embodiments the neurodegenerative disease is Amyotrophic lateral sclerosis. In embodiments the neurodegenerative disease is Huntington's disease. In embodiments the neurodegenerative disease is Parkinson's disease.

In an aspect is provided a method of treating a pulmonary disease, the method including administering to a subject in need thereof (e.g., a subject having the pulmonary disease or a subject who may develop the pulmonary disease) a therapeutically effective amount of a compound described herein, including embodiments, and a β2 adrenergic receptor modulator (e.g., inhibitor, antagonist, inverse agonist, agonist, allosteric modulator, allosteric inhibitor, allosteric antagonist, orthosteric inhibitor, orthosteric antagonist, orthosteric inverse agonist, or orthosteric agonist). In embodiments the pulmonary disease is asthma.

In an aspect is provided a method of treating a cardiovascular disease, the method including administering to a subject in need thereof (e.g., a subject having the cardiovascular disease or a subject who may develop the cardiovascular disease) a therapeutically effective amount of a compound described herein, including embodiments, and a β2 adrenergic receptor modulator (e.g., inhibitor, antagonist, inverse agonist, agonist, allosteric modulator, allosteric inhibitor, allosteric antagonist, orthosteric inhibitor, orthosteric antagonist, orthosteric inverse agonist, or orthosteric agonist). In embodiments the cardiovascular disease in hypertension. In embodiments the cardiovascular disease is heart failure.

In embodiments, the method includes administering a β2 adrenergic receptor modulator. In embodiments, the method includes administering a β2 adrenergic receptor inhibitor. In embodiments, the method includes administering a β2 adrenergic receptor antagonist. In embodiments, the method includes administering a β2 adrenergic receptor inverse agonist. In embodiments, the method includes administering a β2 adrenergic receptor agonist. In embodiments, the method includes administering a β2 adrenergic receptor allosteric modulator. In embodiments, the method includes administering a β2 adrenergic receptor allosteric inhibitor. In embodiments, the method includes administering a β2 adrenergic receptor allosteric antagonist. In embodiments, the method includes administering a β2 adrenergic receptor orthosteric inhibitor. In embodiments, the method includes administering a β2 adrenergic receptor orthosteric antagonist. In embodiments, the method includes administering a β2 adrenergic receptor orthosteric inverse agonist. In embodiments, the method includes administering a β2 adrenergic receptor orthosteric agonist

### V. Embodiments

The definitions for variables R¹, R², R³, R⁴, X¹, and X² that are found in this section, only apply to the aspect and embodiments in this section (Section titled Embodiments), and not to the other sections of the application (e.g., other sections of description, examples, figures, claims, or aspects and embodiments found outside the present Section titled Embodiments).

In an aspect is provided a compound having the formula:

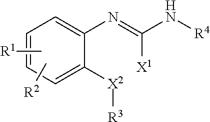

where R⁴ is independently substituted or unsubstituted aryl, or substituted or unsubstituted heteroaryl, substituted or unsubstituted cycloalkyl, substituted or unsubstituted spirocycloalkyl, substituted or unsubstituted heterocycloalkyl, independently hydrogen, or substituted or unsubstituted alkyl, and where R¹ and R² are independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —CHF₂, —CH₂F, OCF₃, —OCHF₂, substituted or unsubstituted (C₁-C₅) alkyl, substituted or unsubstituted heteroalkyl, substituted or unsubstituted cycloalkyl, or substituted or unsubstituted heterocycloalkyl substituted or unsubstituted aryl, or substituted or unsubstituted heteroaryl, and where R³ is —H, —NH₂, —OH, —O-alkyl, —N-alkyl, —N-cycloalkyl, —N-dialkyl, -alkyl, —CN, —CF₃, —NO₂, —COOH, or —NHC(═NH)NH₂, and where X¹ and X² are independently N, CH or C.

Embodiment P1. A compound having the formula:

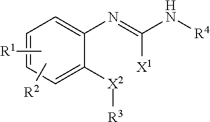

wherein
R⁴ is independently substituted or unsubstituted aryl, or substituted or unsubstituted heteroaryl, substituted or unsubstituted cycloalkyl, substituted or unsubstituted spirocycloalkyl, substituted or unsubstituted heterocycloalkyl, independently hydrogen, substituted or unsubstituted alkyl;
R¹ and R² are independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —CHF₂, —CH₂F, OCF₃, —OCHF₂, substituted or unsubstituted (C₁-C₅) alkyl, substituted or unsubstituted heteroalkyl, substituted or unsubstituted cycloalkyl, or substituted or unsubstituted heterocycloalkyl substituted or unsubstituted aryl, or substituted or unsubstituted heteroaryl;
R³ is —H, —NH₂, —OH, —O-alkyl, —N-alkyl, —N-cycloalkyl, —N-dialkyl, -alkyl, —CN, —CF₃, —NO₂, —COOH, or —NHC(═NH)NH₂, and
wherein X¹ and X² are independently N or C.

Embodiment β2. The compound of embodiment P1, wherein R⁴ is substituted or unsubstituted phenyl, substituted or unsubstituted naphthyl, substituted or unsubstituted pyridinyl or substituted or unsubstituted pyrimidinyl.

Embodiment P3. The compound of embodiment P1, wherein X² is C and R³ is NH₂.

Embodiment P4. The compound of embodiment P1, wherein X¹ is C and X² is N.

### VI. Additional Embodiments

Embodiment 1. A compound having the formula:

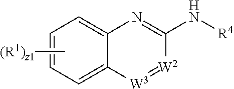

wherein
R¹ is independently halogen, —CX³₃, —CHX³₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
W² is N, CH, or C(R²);
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W³ is N, CH, or C(R³);
R³ is independently halogen, —CX³₃, —CHX³₂, —CH₂X³, —OCX³₃, —OCH₂X³, —OCHX³₂, —CN, —SOₙ₁R³⁰, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently substituted or unsubstituted aryl, substituted or unsubstituted heteroaryl, substituted or unsubstituted cycloalkyl, substituted or unsubstituted spirocycloalkyl, substituted or unsubstituted heterocycloalkyl, hydrogen, substituted or unsubstituted alkyl, or substituted or unsubstituted heteroalkyl;
R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; and R^{3A} and R^{3B} substituents bonded to the same nitrogen atom may optionally be joined to form a substituted or unsubstituted heterocycloalkyl or substituted or unsubstituted heteroaryl;
X, X¹, X², and X³ are independently —F, —Cl, —Br, or —I;
n1, n2, and n3 are independently an integer from 0 to 4; and
m1, m2, m3, v1, v2, and v3 are independently 1 or 2.

Embodiment 2. A compound having the formula:

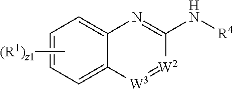

wherein
R¹ is independently halogen, —CX³₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
W² is N, CH, or C(R²);
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W³ is N, CH, or C(R³);
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently substituted or unsubstituted aryl, substituted or unsubstituted heteroaryl, substituted or unsubstituted cycloalkyl, substituted or unsubstituted spirocycloalkyl, substituted or unsubstituted heterocycloalkyl, hydrogen, or substituted or unsubstituted alkyl;
R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; and R^{3A} and R^{3B} substituents bonded to the same nitrogen atom may optionally be joined to form a substituted or unsubstituted heterocycloalkyl or substituted or unsubstituted heteroaryl;
X, X¹, X², and X³ are independently —F, —Cl, —Br, or —I;
n1, n2, and n3 are independently an integer from 0 to 4; and
m1, m2, m3, v1, v2, and v3 are independently 1 or 2.

Embodiment 3. The compound of embodiments 1 to 2, wherein R⁴ is substituted or unsubstituted phenyl, substituted or unsubstituted naphthyl, substituted or unsubstituted pyridinyl or substituted or unsubstituted pyrimidinyl.

Embodiment 4. The compound of one of embodiments 1 to 3, having the formula:

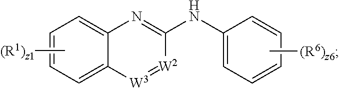

wherein
R⁶ is independently halogen, —CX⁶₃, —CHX⁶₂, —CH₂X⁶, —OCX⁶₃, —OCH₂X⁶, —OCHX⁶₂, —CN, —SOₙ₃R⁶⁰, —SOᵥ₃NR^{6A}R^{6B}, —NHC(O)NR^{6A}R^{6B}, —N(O)ₘ₃, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 5;
R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
X⁶ is independently —F, —Cl, —Br, or —I;
n6 is independently an integer from 0 to 4; and
m6 and v6 are independently 1 or 2.

Embodiment 5. The compound of one of embodiments 1 to 4, wherein W² is N.

Embodiment 6. The compound of one of embodiments 1 to 5, wherein W³ is C(R³).

Embodiment 7. The compound of one of embodiments 1 to 6, wherein R³ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment 8. The compound of one of embodiments 1 to 6, wherein R³ is independently —NH₂, —OH, —O-alkyl, —N-alkyl, —N-cycloalkyl, —N-dialkyl, unsubstituted C₁-C₄ alkyl, —CN, —CF₃, —NO₂, —COOH, or —NHC(═NH)NH₂.

Embodiment 9. The compound of one of embodiments 1 to 6, wherein R³ is independently —NH₂.

Embodiment 10. The compound of one of embodiments 1 to 9, wherein z1 is 1.

Embodiment 11. The compound of one of embodiments 3 to 9, having the formula:

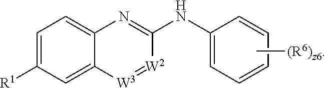

Embodiment 12. The compound of one of embodiments 1 to 11, wherein R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment 13. The compound of one of embodiments 1 to 11, wherein R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, unsubstituted C₁-C₄ alkyl, unsubstituted phenyl, or unsubstituted 5 to 6 membered heteroaryl.

Embodiment 14. The compound of one of embodiments 1 to 11, wherein R¹ is independently halogen, —CF₃, unsubstituted C₁-C₄ alkyl, or unsubstituted phenyl.

Embodiment 15. The compound of one of embodiments 1 to 11, wherein R¹ is independently halogen or —CF₃,

Embodiment 16. The compound of one of embodiments 1 to 11, wherein R¹ is independently —Cl, —Br, —I, or —CF₃,

Embodiment 17. The compound of one of embodiments 3 to 16, wherein R⁶ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₁₀ alkyl, substituted or unsubstituted 2 to 10 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl.

Embodiment 18. The compound of one of embodiments 3 to 16, wherein R⁶ is independently —CH₂OH, —CH₂CH₂COOH, —CH₂CH₂COOCH₂CH(OH)CH₂OH, —SO₂NH₂, —C(O)NHCH₃, —C(O)CH₃, —C(O)OCH₃, or —OH.

Embodiment 19. The compound of one of embodiments 1 to 18, wherein z6 is 1.

Embodiment 20. The compound of one of embodiments 1 to 18, wherein z6 is 0.

Embodiment 21. The compound of one of embodiments 1 to 18, having the formula:

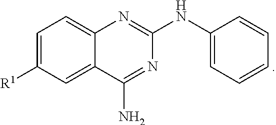

Embodiment 22. The compound of embodiments 1 or 2, having the formula:

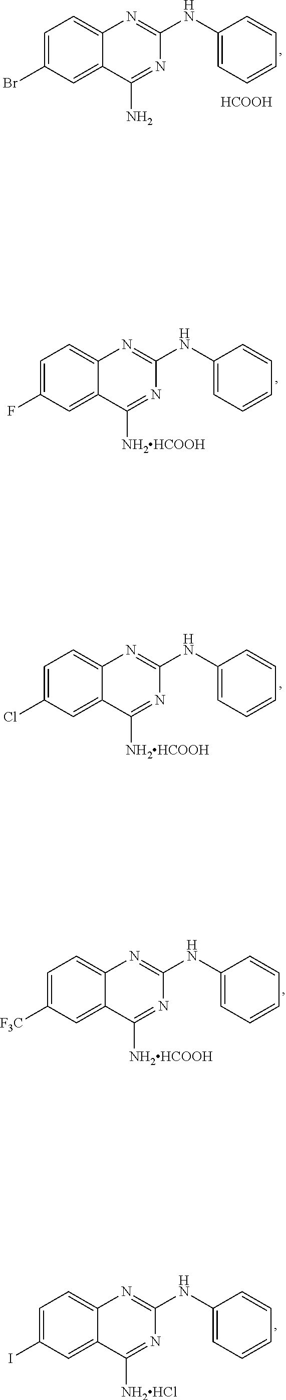

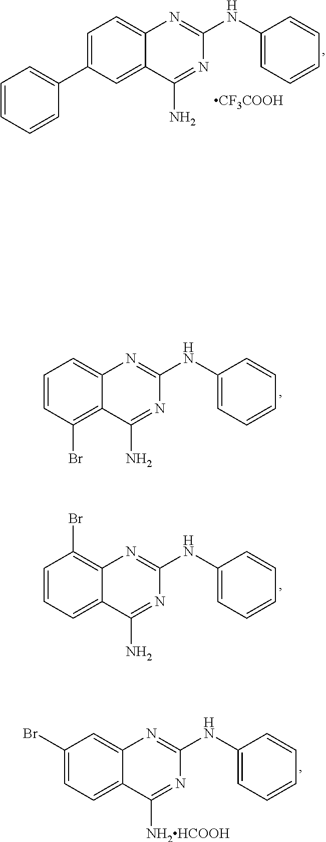

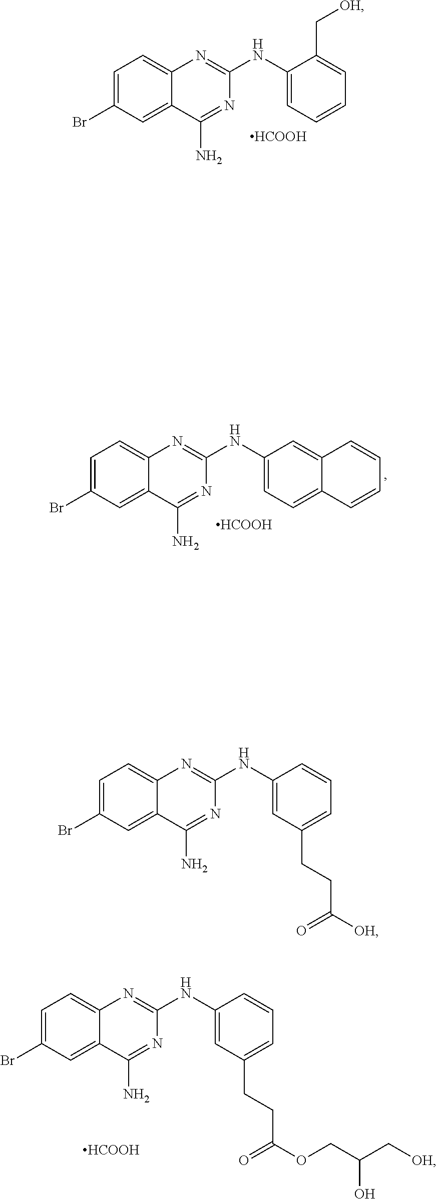

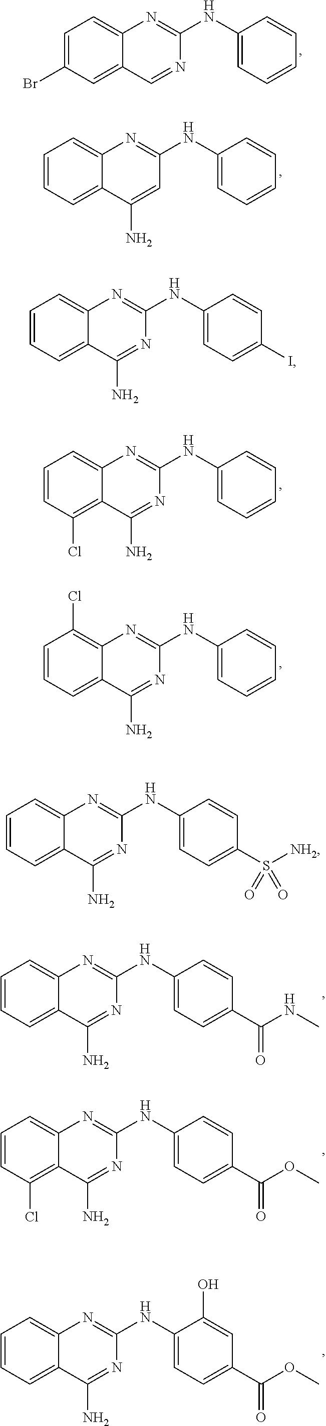

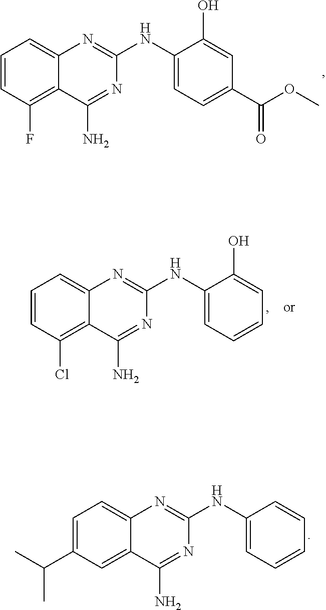

Embodiment 23. A pharmaceutical composition comprising a compound of one of claims 1 to 22 and a pharmaceutically acceptable excipient.

Embodiment 24. The pharmaceutical composition of embodiment 23, further comprising a second agent, wherein the second agent is a β2 adrenergic receptor inhibitor.

Embodiment 25. A method of treating a disease associated with β2 adrenergic receptor, said method comprising administering to a subject in need thereof a therapeutically effective amount of a compound of one of embodiments 1 to 22.

Embodiment 26. A method of treating Parkinson's disease, hypertension, heart failure, asthma, myocardial infarction, angina pectoris, tachycardia, anxiety, tremor, migraine headache, cluster headache, hyperhidrosis, glaucoma, thyrotoxicosis, hyperthyroidism, esophageal variceal, ascites, post-traumatic stress disorder, psychogenic polydispsia, hemangioma, or cardiomyopathy, said method comprising administering to a subject in need thereof a therapeutically effective amount of a compound of one of embodiments 1 to 22.

Embodiment 27. The method of one of embodiments 25 to 26, further comprising administering a second agent to the subject in need thereof, wherein the second agent is a β2 adrenergic receptor inhibitor.

### EXAMPLES

### Example 1: Discovery of an Allosteric Modulator Binding to a Conformational Hub in the β₂AR

The majority of drugs acting on G protein-coupled receptors (GPCRs) target the orthosteric binding pocket, the site of action of the native hormone or neurotransmitter. There is much interest in finding allosteric ligands for these targets because they modulate physiologic signaling and promise to be more selective than orthosteric ligands that occupy the native ligand binding pocket. For instance, the family of nine adrenergic receptors (ARs) all recognize adrenaline and noradrenaline, and all share high orthosteric site similarity. Here we describe a negative allosteric modulator of the β2 adrenergic receptor (β₂AR), AS408, that binds to the membrane-facing surface of transmembrane segments (TM) 3 and 5, as revealed by X-ray crystallography. AS408 disrupts a water-mediated polar network involving E122^{3.41} and the backbone carbonyls of V210^{5.49} and S207^{5.46}. The AS408 binding site is adjacent to a previously identified molecular switch for β₂AR activation formed by I^{3.40}, P^{5.50} and F^{6.44}. The crystal structure reveals how AS408 stabilizes the inactive conformation of this switch. Consistent with the importance of this region for signal propagation across the membrane, mutagenesis studies reveal that the AS408 binding pocket has strong allosteric coupling to the orthosteric binding pocket, and to the cytoplasmic arrestin and G protein coupling interface.

The orthosteric binding pockets of GPCRs within subfamilies that bind to the same hormones or neurotransmitters, such as the adrenergic and muscarinic receptors, share a high degree of amino acid identity. As a consequence, it is often difficult to develop subtype selective drugs targeting the orthosteric binding pocket. Allosteric modulators bind outside of the highly conserved orthosteric sites and may therefore be more subtype selective. Additionally, allosteric ligands function by modulating responses to native hormones and neurotransmitters, and may therefore be better tolerated. Muscarinic receptors represent a model system for studying allosteric regulation of GPCR function, as numerous allosteric modulators have been described and extensively characterized (7-3). In contrast, only one small-molecule allosteric modulator has been described for beta adrenergic receptors. Cmpd-15, a negative allosteric modulator (NAM), was shown to bind to the intracellular surface of the β2 adrenergic receptor (β₂AR) in a pocket formed by the cytoplasmic ends of transmembrane segments (TMs) 1, 2, 6 and 7 (4,5). Cmpd-6 is a 611 Da positive allosteric modulator that binds to a pocket formed by intracellular loop 2 (ICL2) and the cytoplasmic ends of transmembrane segments (TMs) 3 and 4. Allosteric modulators for β adrenergic receptors (βARs) would have therapeutic use in several disease entities including hypertension, Parkinson's disease and heart failure. We therefore explored the use of in silico docking to identify an allosteric modulator for the β₂AR.

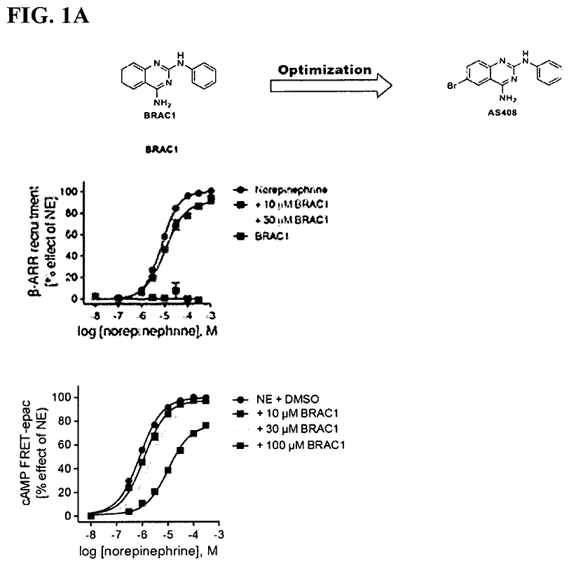

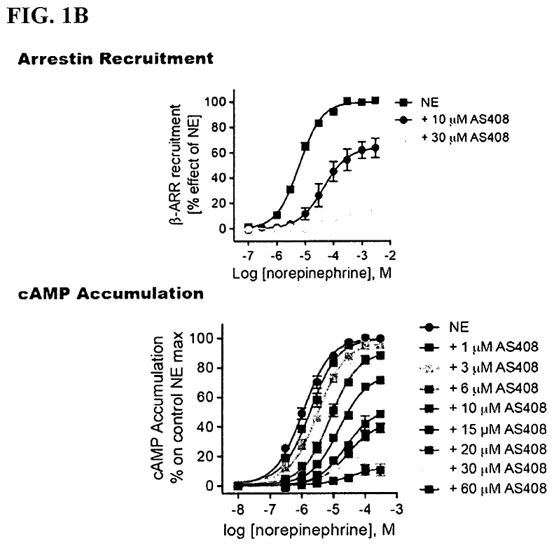

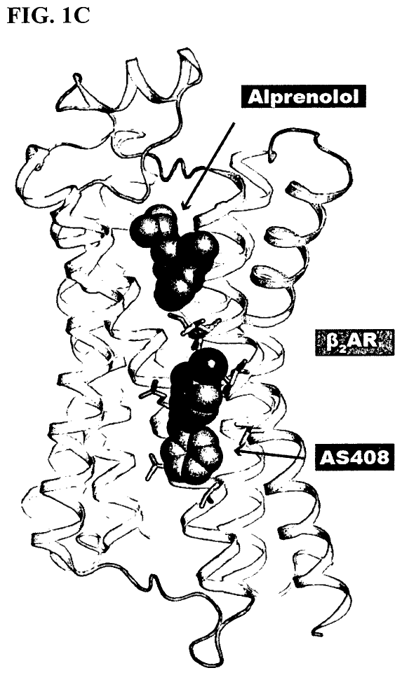

We previously reported a crystal structure of the active state of the M2 muscarinic receptor with a positive allosteric modulator (LY2119620) bound in the extracellular vestibule (I). In an effort to identify allosteric modulators for the β₂AR, we performed in silico docking using the extracellular vestibule of the β₂AR as a template. One of the initial docking hits, BRAC1, (FIG. 1A) exhibited weak, negative allosteric regulation of arrestin recruitment and cAMP accumulation. Efforts to obtain a crystal structure of the β₂AR bound to BRAC1 were unsuccessful; however, we generated a more potent brominated derivative (AS408, FIG. 1B), and were able to obtain a crystal structure of the β₂AR bound to this negative allosteric modulator (NAM).

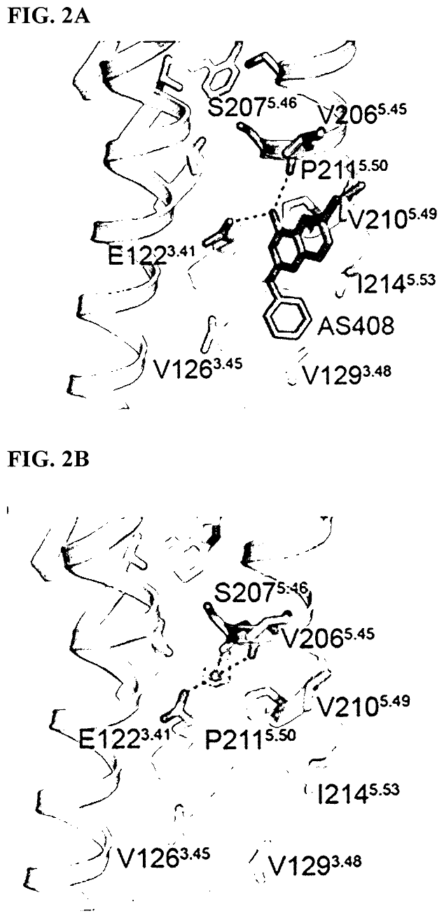

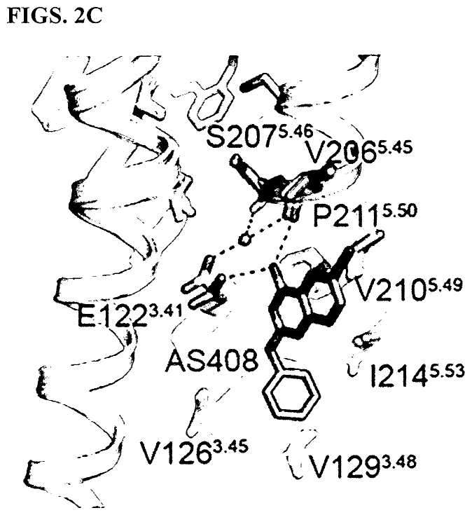

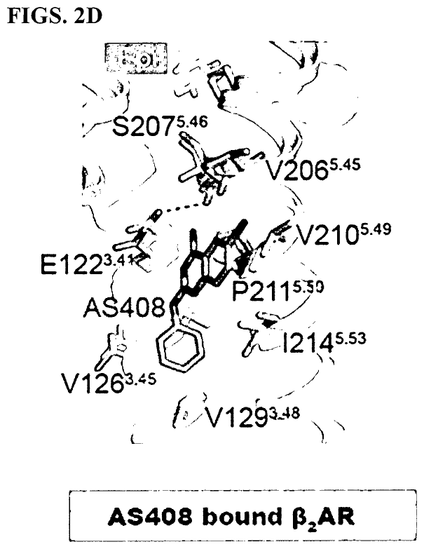

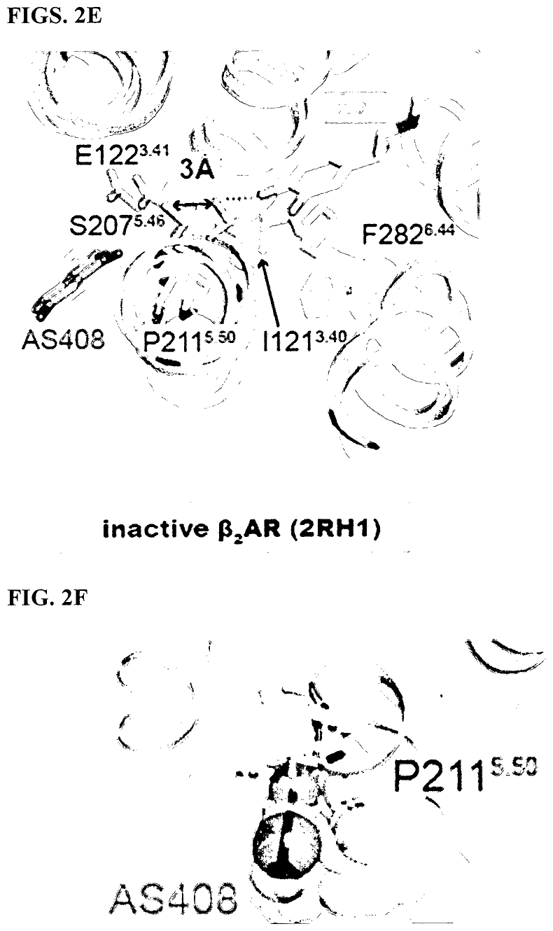

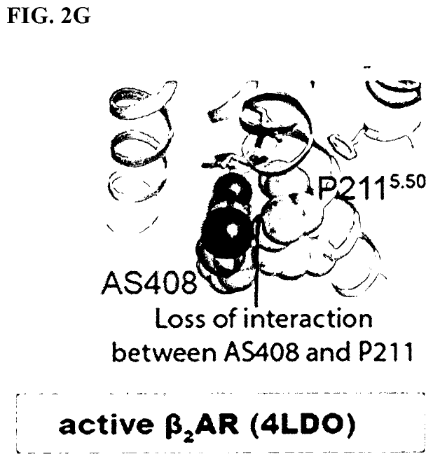

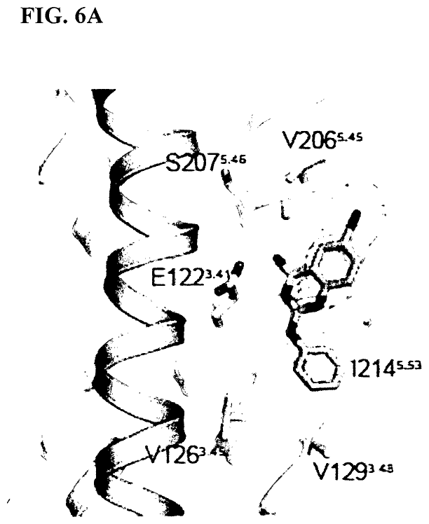

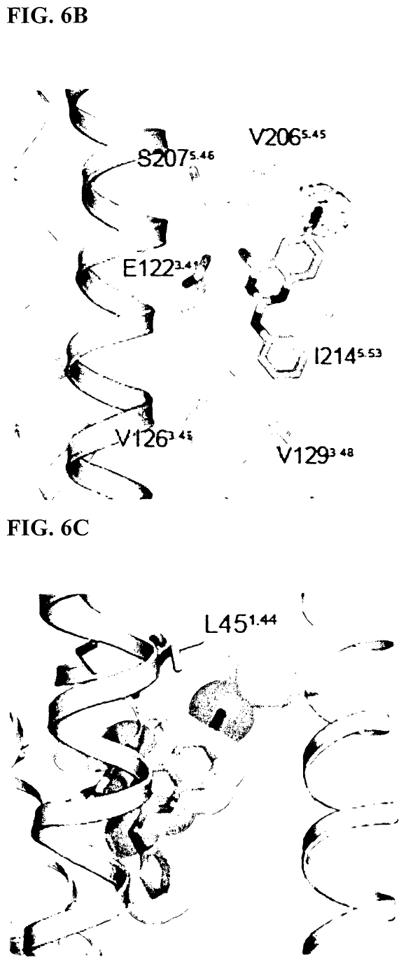

Crystals were obtained in lipidic cubic phase with the β₂AR bound to the neutral antagonist alprenolol and AS408. The structure was solved by molecular replacement at 3.1 Å (Methods and Table 1). We were surprised to observe well-defined Fo-Fc electron density for AS408 at the membrane facing surface of TM3 and TM5 (FIG. 1C, FIG. 6A), but not in the extracellular vestibule. The position of AS408 was further confirmed by obtaining an anomalous signal for bromine (FIG. 6B). The binding pocket is formed by predominantly hydrophobic interactions with C125^{3.44}, V126^{3.45}, V129^{3.48}, V210^{5.49}, P211^{5.50} and I214^{5.53}. The primary amine of AS408 can hydrogen bond with E122^{3.41} and the carbonyl of V206^{5.45} (FIG. 2A). It should be noted that L45^{1.44} of an antiparallel symmetry mate interacts with the Br of AS408 in our crystal structure (FIG. 6C).

TABLE 1
Data collection and refinement statistics (molecular replacement).

| | Native | | Anomalous (Br) | |
|---|---|---|---|---|
| Data collection | | | | |
| Wavelength (Å) | 1.00 | | 0.91 | |
| Number of crystals | 81 | | 284 | |
| Space group | P2₁2₁2₁ | | P2₁2₁2₁ | |
| Cell dimensions | | | | |
| a, b, c (Å) | 40.46, 75.71, 173.41 | | 40.46, 75.71, 173.41 | |
| α, β, γ (°) | 90.00, 90.00, 90.00 | | 90.00, 90.00, 90.00 | |
| Resolution (Å) | 49.4-3.1 | (3.2-3.1) * | 50-4.0 | (4.1-4.0) |
| R_{sym} or R_{merge} | 0.21 | (1.11) | 0.32 | (1.80) |
| CC_{1/2} (%) | 99.6 | (68.5) | 92.2 | (95.7) |
| I/σI | 8.46 | (1.27) | 16.07 | (4.20) |
| Completeness (%) | 99.1 | (98.2) | 99.9 | (99.8) |
| Redundancy | 13.3 | (9.7) | 54.6 | (56.0) |

| Refinement | | |
|---|---|---|
| Resolution (Å) | 20-3.1 | |
| No. reflections (test set) | 10122 | (999) |

| R_{work}/R_{free} | 0.251/0.277 |
|---|---|
| No. atoms | |
| Protein | 3523 |
| Alprenolol | 18 |
| AS408 | 19 |
| Others (Lipids, ions, | 56 |
| water) | |
| B-factors | |
| Receptor | 84.0 |
| T4 lysozyme | 102.4 |
| Alprenolol | 73.2 |
| AS408 | 70.4 |
| Others (Lipids, ions, | 100.2 |
| water) | |
| R.m.s. deviations | |
| Bond lengths (Å) | 0.008 |
| Bond angles (°) | 0.750 |
| Ramachandran statistics | |
| Favored regions (%) | 98.86 |
| Allowed regions (%) | 1.14 |
| Outliers (%) | 0 |

* Values in parentheses are for highest-resolution shell.

When comparing the structures of the inactive state β₂AR (pdb 2rh1) with β₂AR bound to AS408 the differences are subtle (FIG. 2C). We observe larger differences in AS408 binding pocket residues when comparing the new structure to an active state structure (FIG. 2D, 2E). The largest change in the orthosteric binding pocket upon agonist activation of the β₂AR is an inward movement of S207^{5.46} (FIG. 2D, 2E). This leads to a rearrangement of the packing interactions between I121^{3.40}, P211^{5.50} and F282^{6.44} and an outward movement of the cytoplasmic end of TM6. As a result of the associated inward movement of P211^{5.50}, it would lose Van der Waals contact with AS408 (FIGS. 2F and 2G). Thus, the complementary interactions between AS408 and the inactive β₂AR stabilize the inactive conformation.

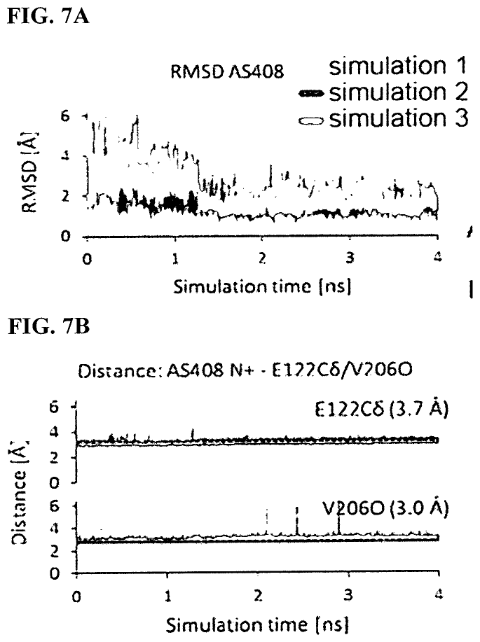

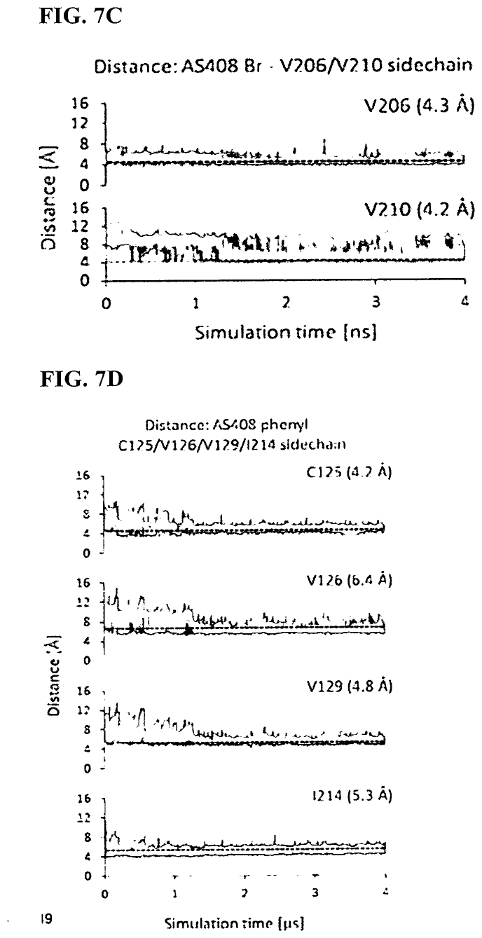

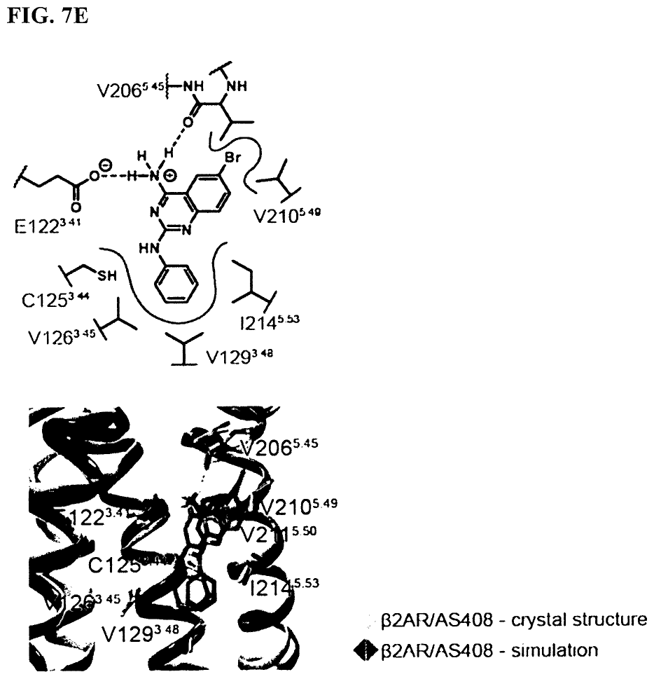

Of interest, P211^{5.50} was shown to be part of an allosteric hub along with I121^{3.40} and F282^{6.44}. binding pocket for AS408 on the β₂AR is relatively shallow when compared to the orthosteric pocket. To assess the stability of interactions between AS408 and the receptor we performed three independent all-atom molecular dynamics simulations that included a DOPC phospholipid bilayer. Independent simulation times are 4 μs each, resulting in an overall simulation time of 12 microseconds. In all three simulations, AS408 adopts a binding mode that is very similar to the crystal structure (FIG. 7A-7E). The interactions of the positively charged amino group of AS408 with the carboxyl group of E122^{3.41} and the backbone oxygen of V206^{5.45} were well maintained throughout the simulation with interaction frequencies of 100 and 98%, respectively. In the absence of AS408, E122 may interact with the backbone oxygens of V206 and S207 through a water mediated hydrogen bond. While not modeled into the deposited inactive-state structure of the β₂AR (pdb 2rh1), there is positive density consistent with a water-mediated hydrogen bond network bridging the carboxylic acid function of E122^{3.41} in TM3 with the backbone carbonyl oxygen of V206^{5.45} and S207^{5.46} (TM5). Of note, activation of the β₂AR involves a 2.4 Å inward movement of the alpha-carbon of S207^{5.46} which would disrupt this network and E122^{3.41} would directly hydrogen bond with the backbone carbonyl oxygen of V206^{5.45}. Upon binding of AS408, the water is displaced by the amine nitrogen of the ligand. The rearrangement of the polar network by AS408 might be expected to stabilize TM5 in an inactive conformation (FIG. 2C).

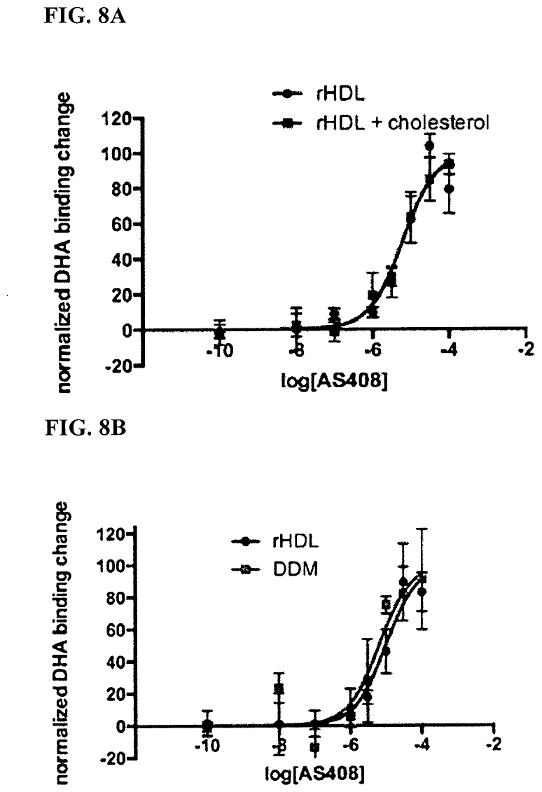

Since the phospholipid bilayer makes a significant contribution to the binding of AS408, we examined the effect of cholesterol and phospholipids on the affinity of AS408. AS408 enhances the binding of ³H dihydroalprenolol (DHA) to purified β₂AR allowing us to determine the effect of a lipid bilayer on the affinity of AS408. The EC50 for the effect of AS408 on DHA binding was similar for β₂AR in detergent, phospholipid or phospholipid with cholesterol, suggesting that lipids do not appear to make a specific contribution to AS408 binding affinity (FIG. 8A-8B).

Functional Properties of AS408

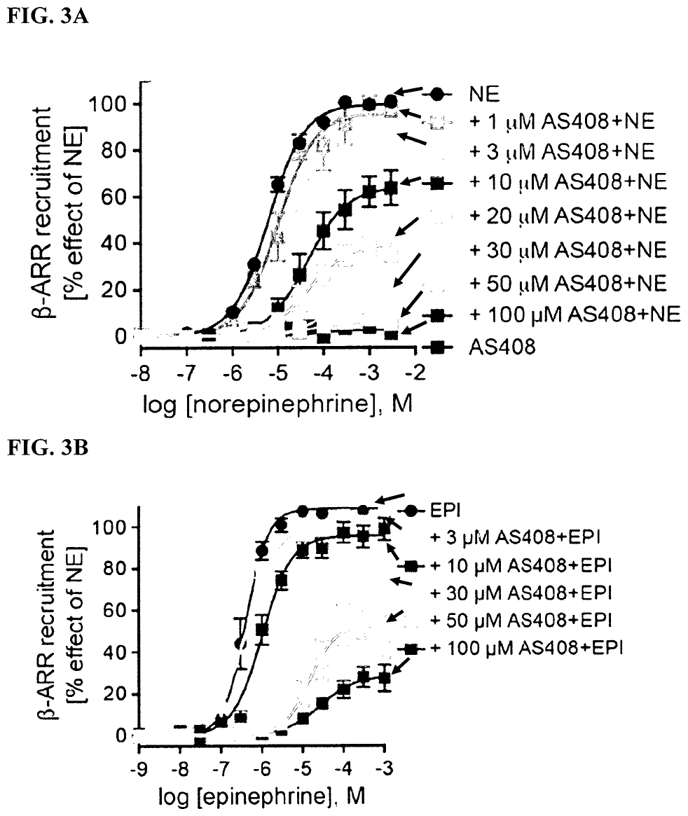

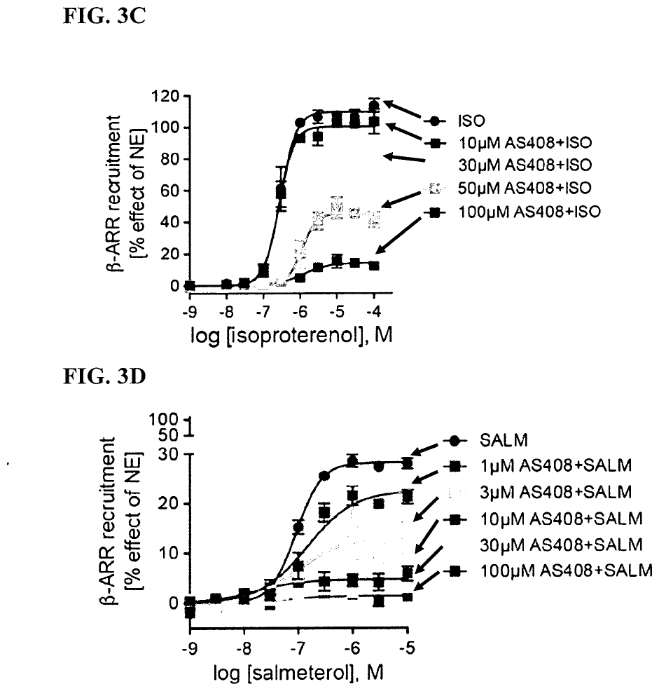

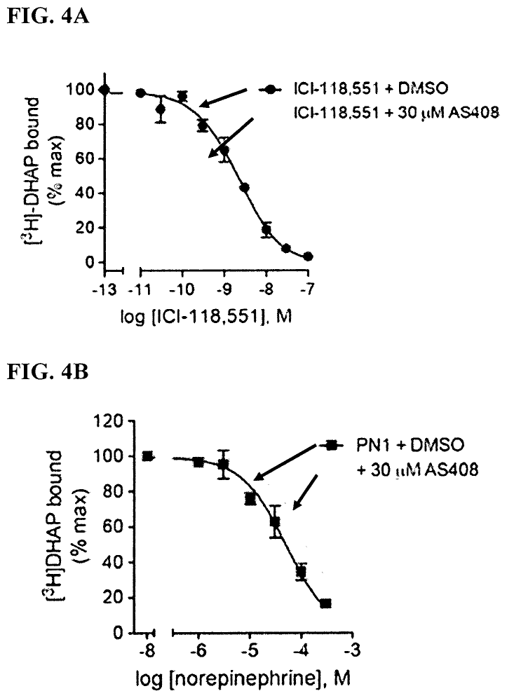

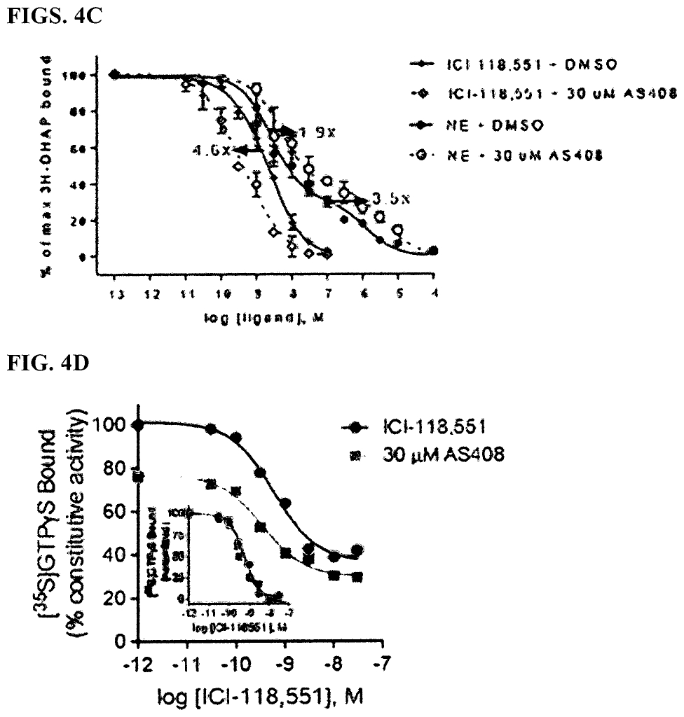

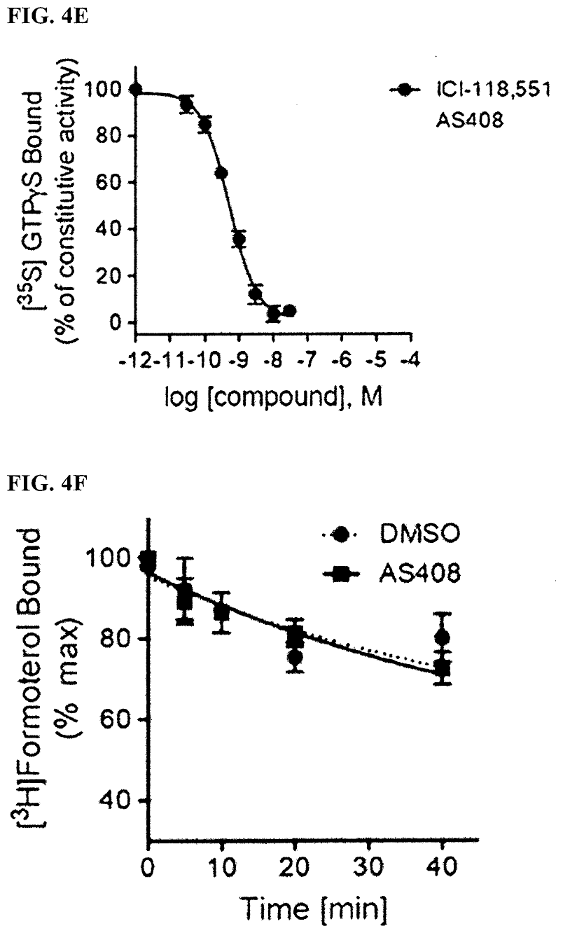

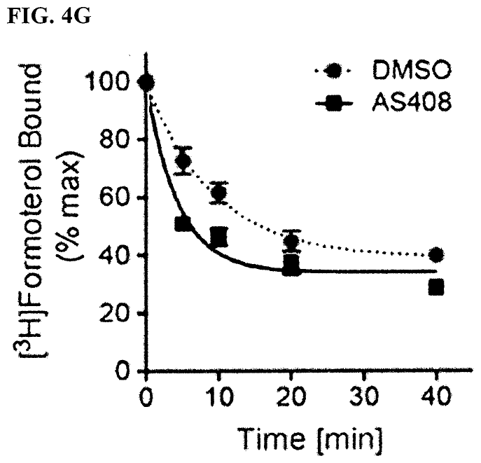

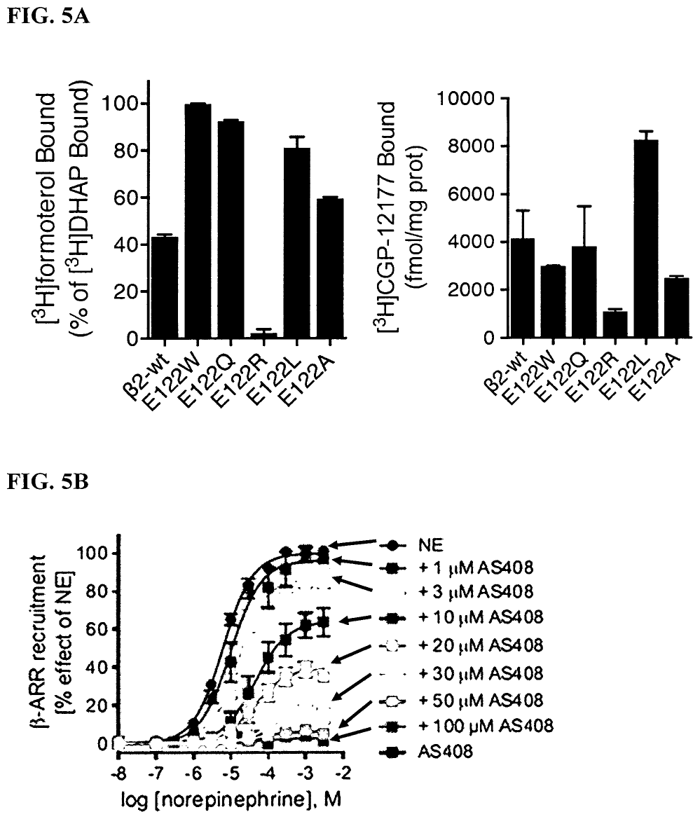

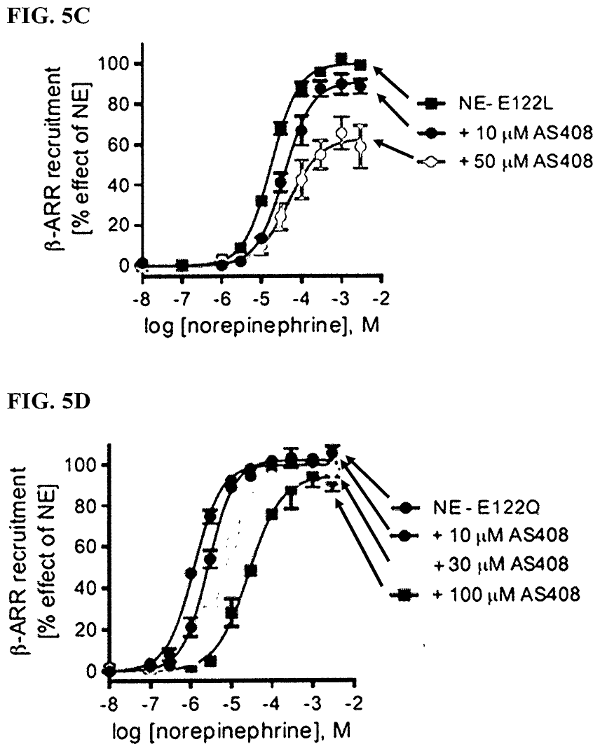

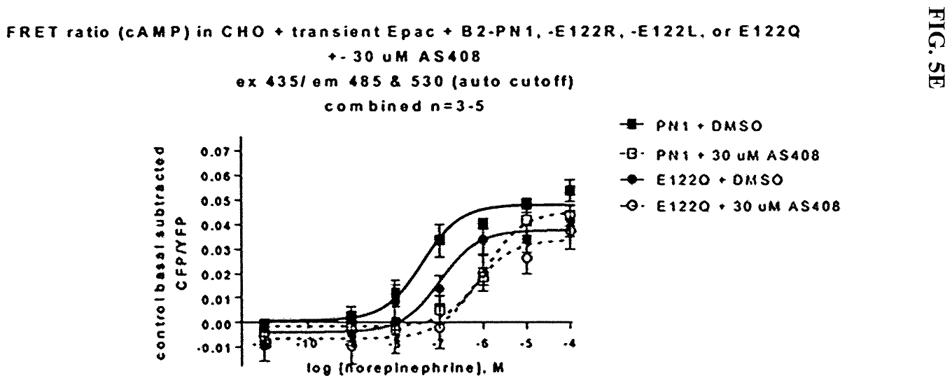

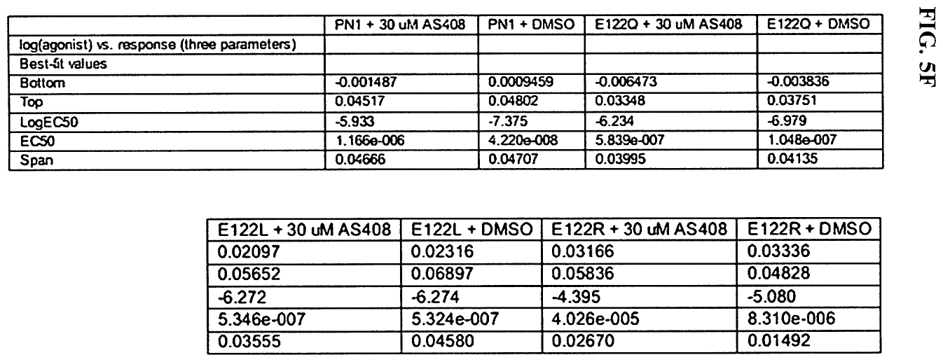

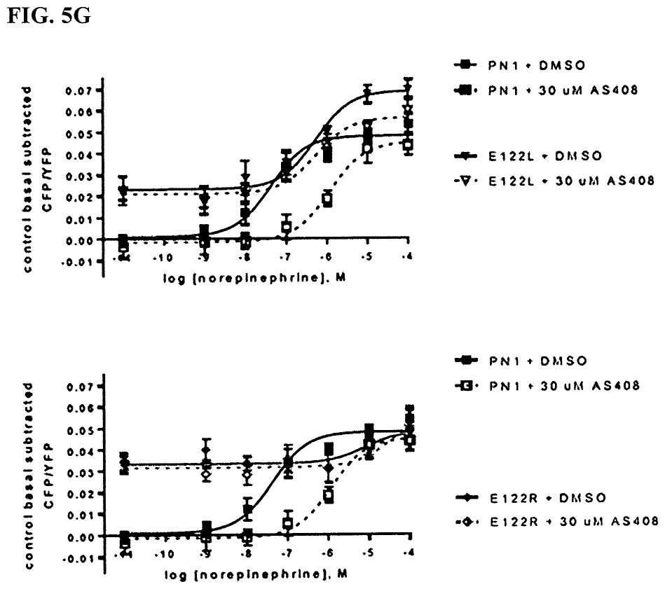

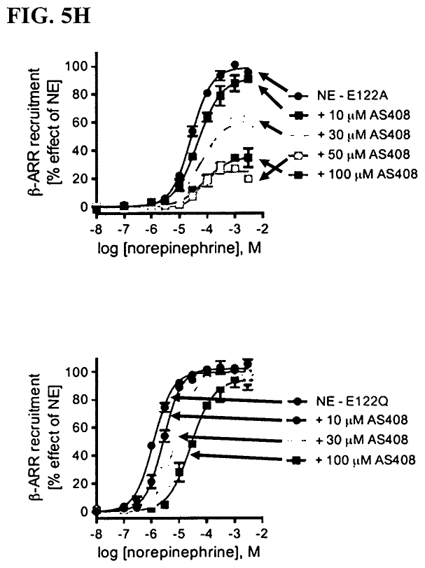

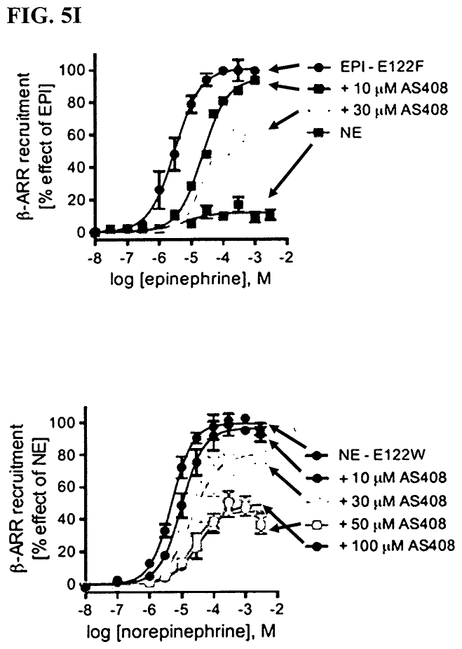

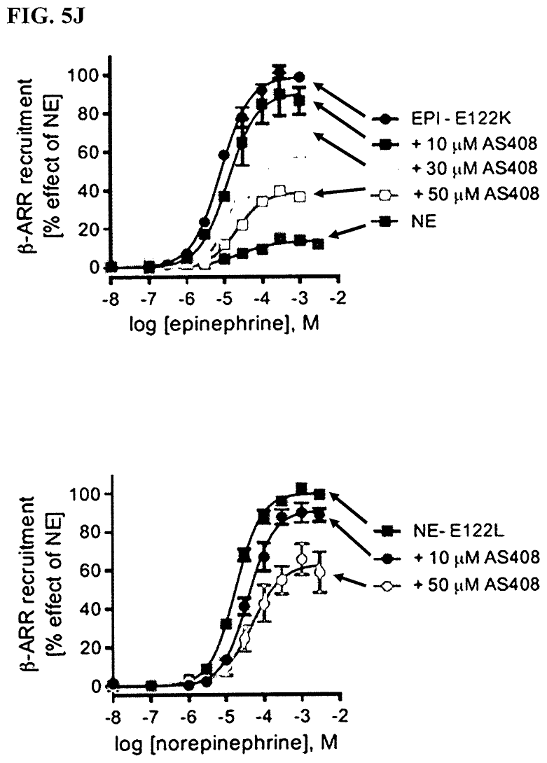

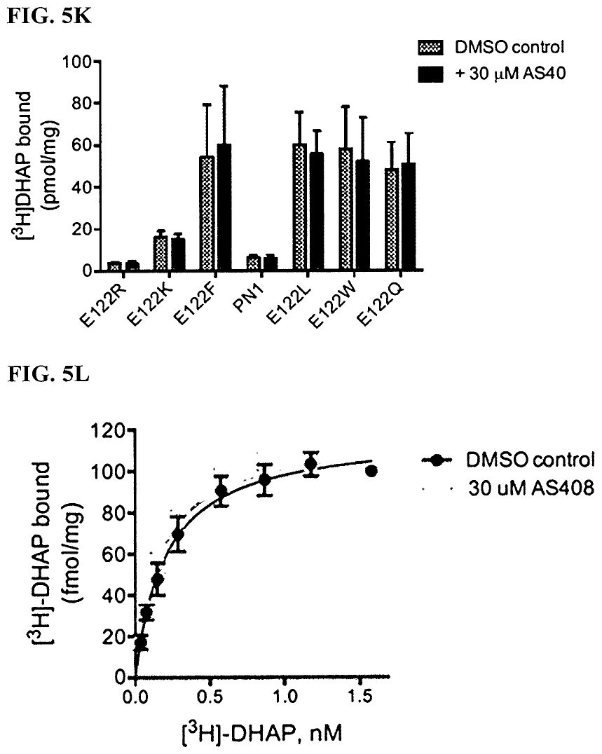

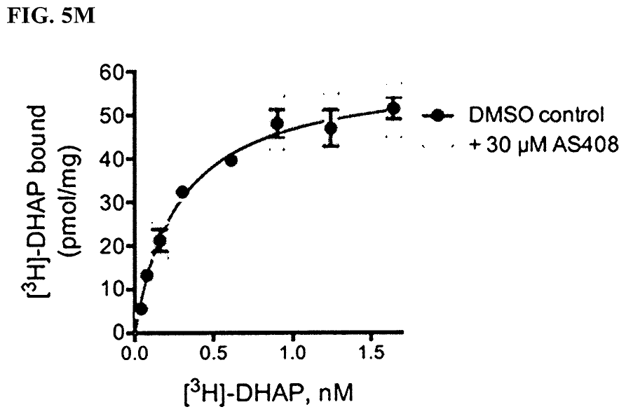

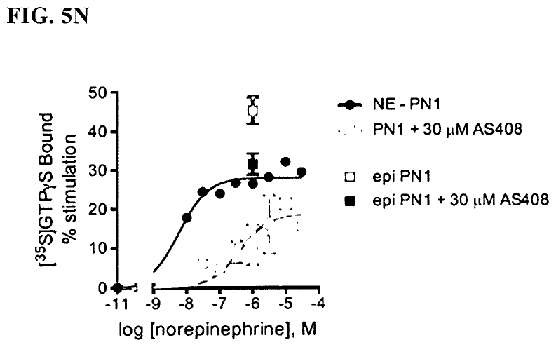

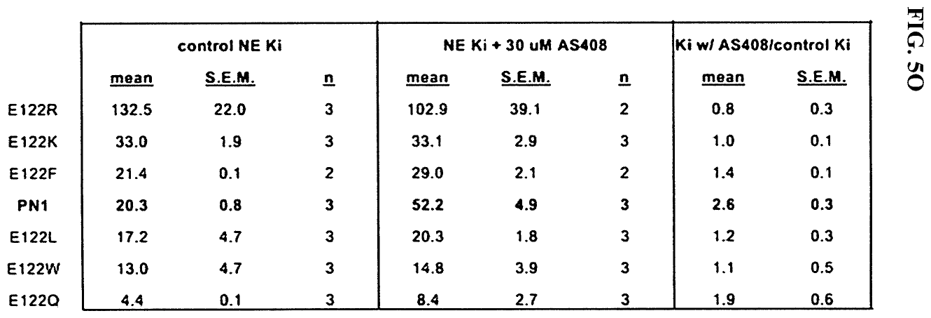

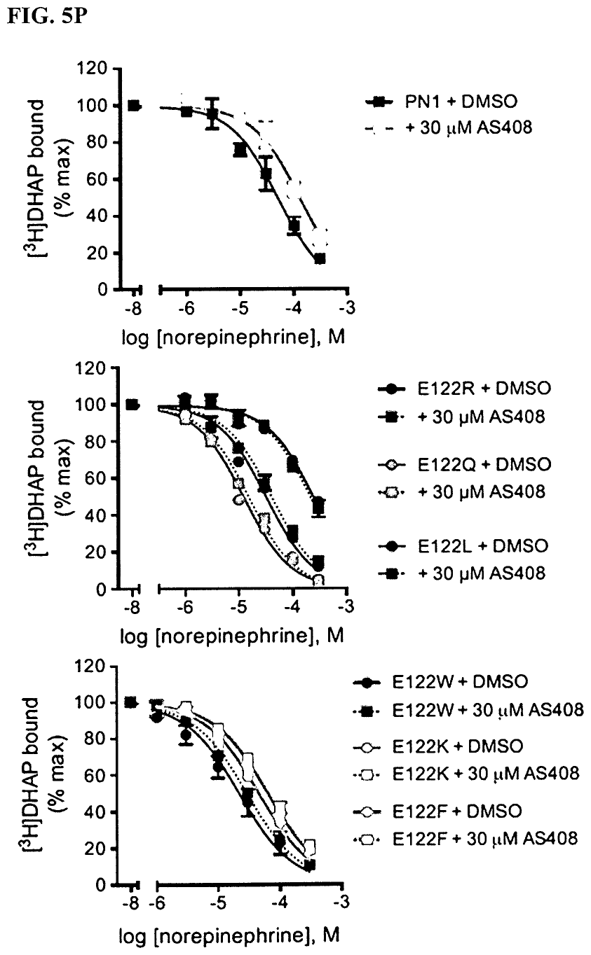

As shown in FIG. 1B, AS408 is a non-biased NAM, having comparable effects on both arrestin recruitment (α=x, Kb=y) and cAMP accumulation (α=0.48, Kb=1.1 μM) based on an operational model of allostery, suggesting negative allosteric activity on orthosteric agonists (α<1) and an accompanying negative effect on orthosteric efficacy (β<1). As shown in FIG. 3A-3D, the efficacy of AS408 is dependent on the efficacy of the orthosteric agonist (FIG. 3A-3D). AS408 has the greatest effect suppressing recruitment of arrestin by the partial agonists norepinephrine and salmeterol. Consistent with its ability to stabilize the inactive state, AS408 enhances the affinity of the β₂AR for the inverse agonist ICI118551 by 4.6-fold (FIG. 4A), and reduces its affinity for the agonist norepinephrine (FIG. 4B). Of interest, AS408 appears to have a greater effect on the affinity of agonist for uncoupled β₂AR (3.5-fold reduction in K_{low}, the low affinity state) compared with Gs-coupled β₂AR (1.9-fold reduction in K_{low}, the high affinity state) (FIG. 4C). AS408 enhances the inhibition of basal activity by ICI118551 (FIG. 4D), and has weak inverse agonist activity by itself (FIG. 4E). [Effect of AS408 on DHA dissociation] Consistent with this observation on equilibrium binding affinity, AS408 had no effect on the dissociation rate of ³H-formoterol in Gs-coupled β₂AR (FIG. 4F), but accelerated the dissociation rate of ³H-formoterol from uncoupled β₂AR in the presence of GTPγS (FIG. 4G).

Structure-Activity of Select AS408 Analogs

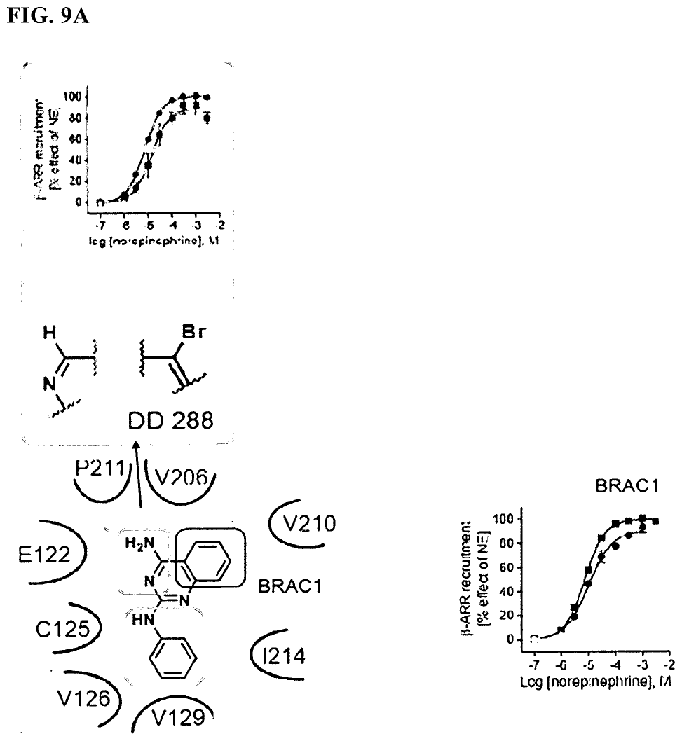

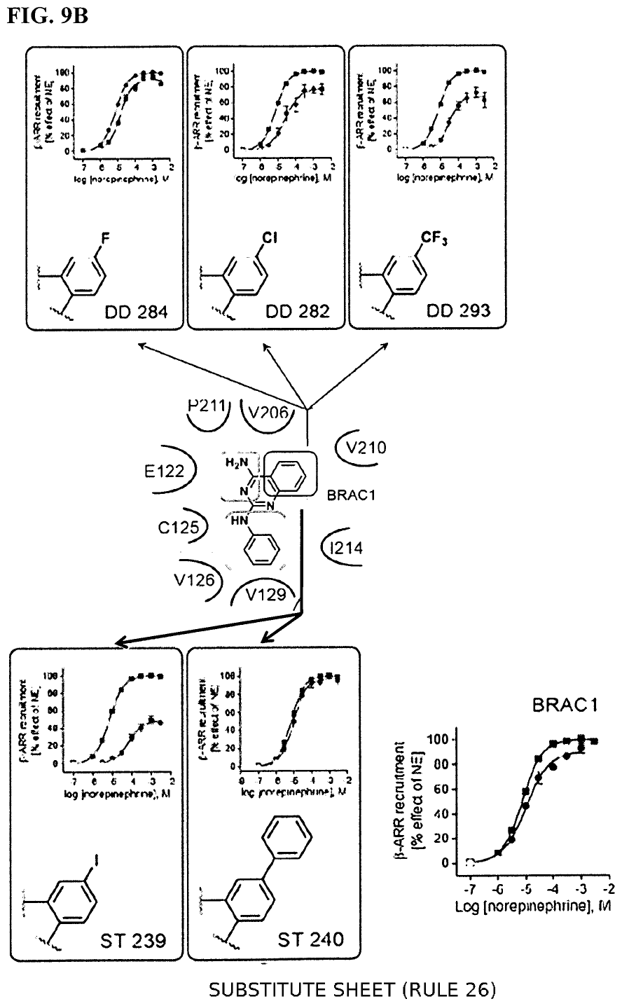

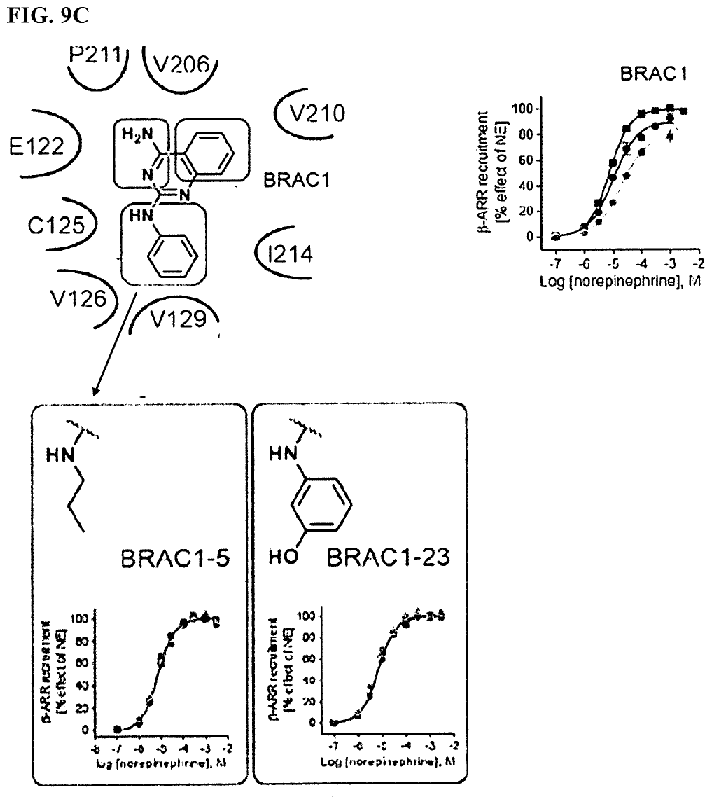

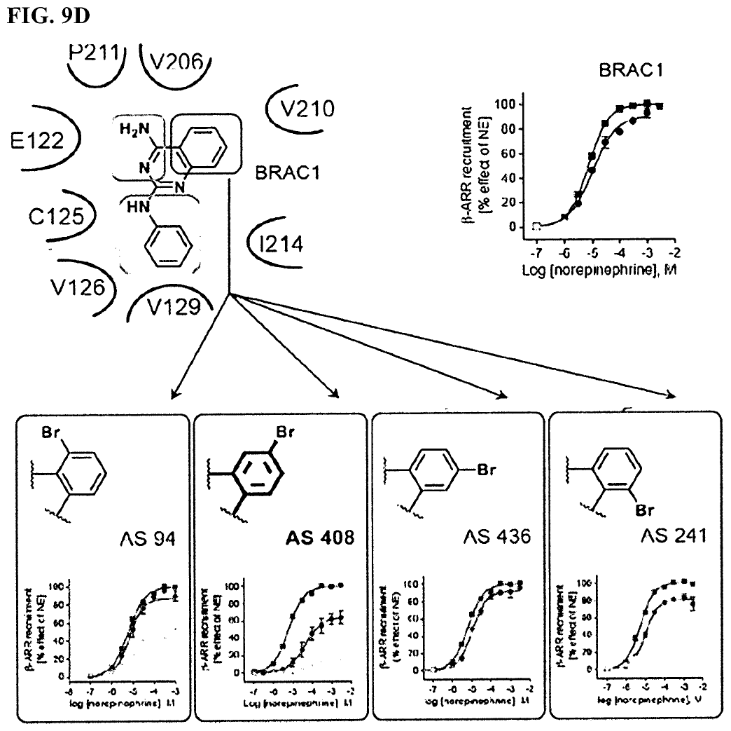

In the process of going from BRAC1, the initial in silico screening hit, to AS408, a number of analogs were generated and tested. FIG. 9A-9D shows how structural differences in these compounds influenced their functional properties. The protonated primary amino group of AS408 forms an ionic interaction and a hydrogen bond to the carboxylate of E122^{3.41} (TM3) and the backbone oxygen of V206^{5.45} (TM5), respectively (FIG. 2C). DS288, missing the amino function, can no longer replace the mediating water molecule linking E122^{3.41} and V206^{5.45} and S207^{5.46} resulting in an attenuated negative allosteric effect. According to the crystal structure, the heterocyclic quinazoline ring of AS408 engages in hydrophobic interactions with the aliphatic moieties of V210^{5.49} and P211^{5.50}. The stronger allosteric effect of AS408, compared to the initial hit BRAC1, can be explained by attractive interactions of the bromo substituent with the highly hydrophobic lipid protein interface. The halogen atom fits nicely between the side chains of V206^{5.45} and V210^{5.49}, when the bromine is located in position 6. In contrast, a bromo substituent in position 5, 7 or 8, of the quinazoline ring were expected to show a less complementary shape (AS436, AS241) or a clash with V206^{5.45} (AS94). As expected, reduced allosteric modulation was observed for these regioisomers. To further probe the effect of the substituent in position 6, we replaced the bromo atom by a set of different (pseudo)halogens. Interestingly, the extent of the hydrophobic interaction to V206^{5.45} and V210^{5.49} increases with the size of the (pseudo)halogen substituent (F≤≤Cl≤CF₃≈Br≈I). While —Cl, —CF3 and —I substituents (DD282, DD293 and ST239 respectively) had activity comparable to Br, the smaller F substituent (DD284) was less potent. Further increasing the hydrophobic substituent by introduction of a phenyl group (ST240) results in further disruption of the negative allosteric effect, suggesting possible repulsive interactions with the side chain of V206^{5.45}. The phenyl ring of AS408 fits into a complementary hydrophobic pocket formed by C125^{3.44}, V126^{3.45}, V129^{3.48} and I214^{5.53}. Starting from BRAC1, replacement of the phenyl group by a smaller aliphatic propyl chain reduces the hydrophobic interactions and reduces the negative allosteric effect (BRAC1-5). Reduction of the allosteric effect was also observed when we introduced a hydroxyl group to the phenyl ring inflicting repulsive interactions at the hydrophobic membrane protein interface (BRAC1-23).

Subtype Selectivity

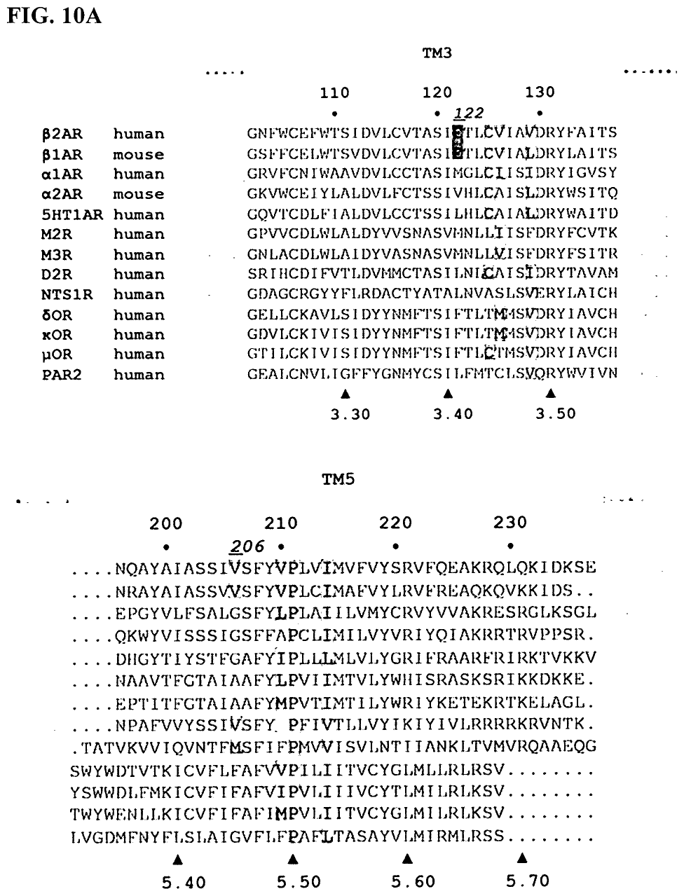

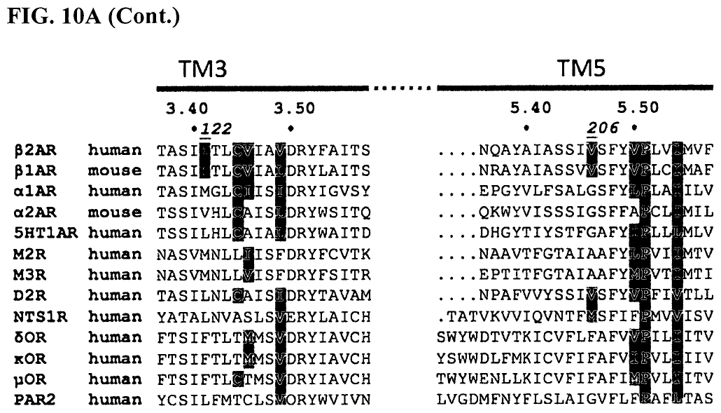

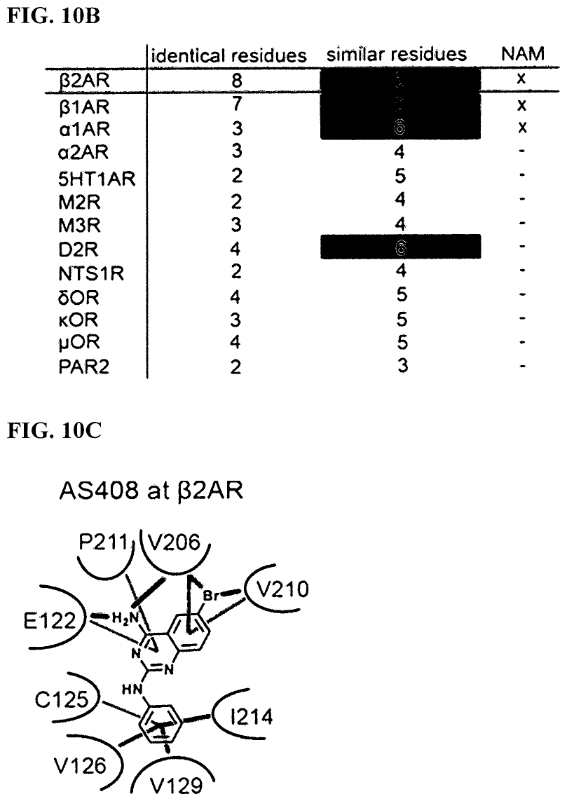

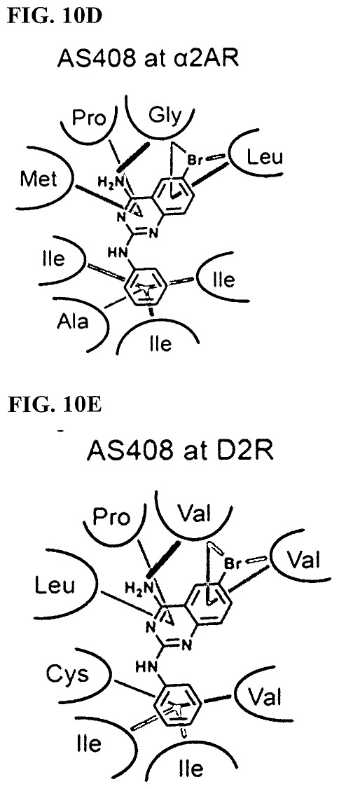

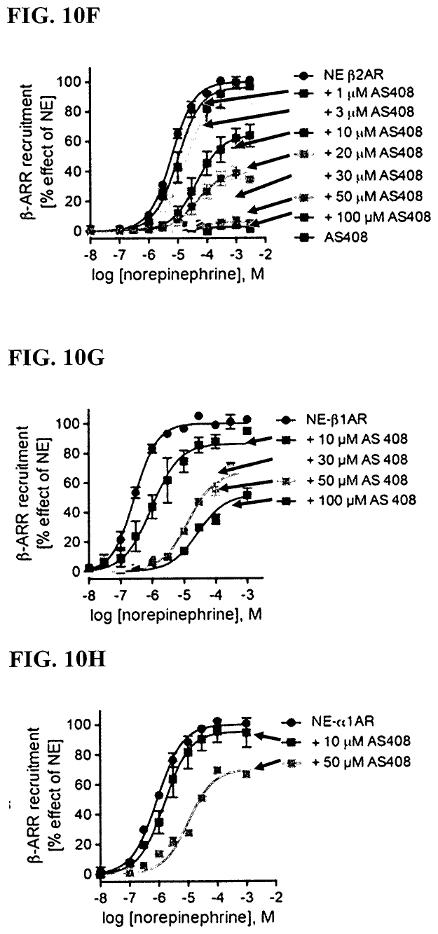

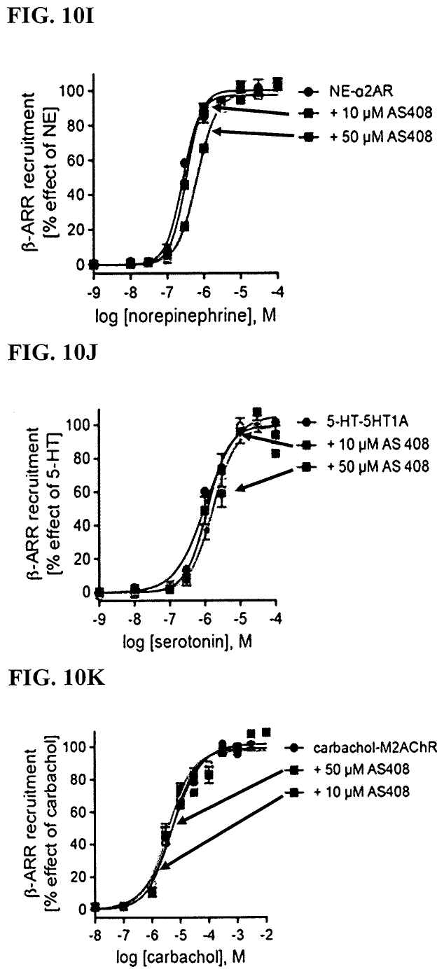

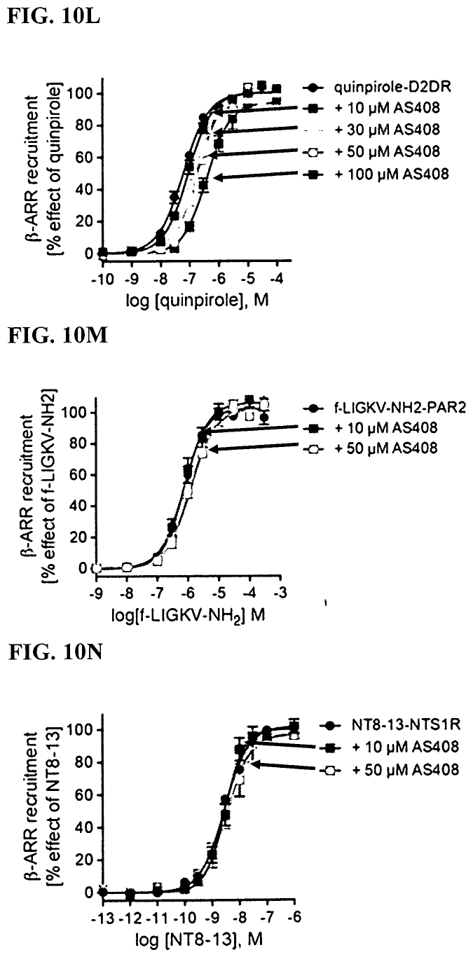

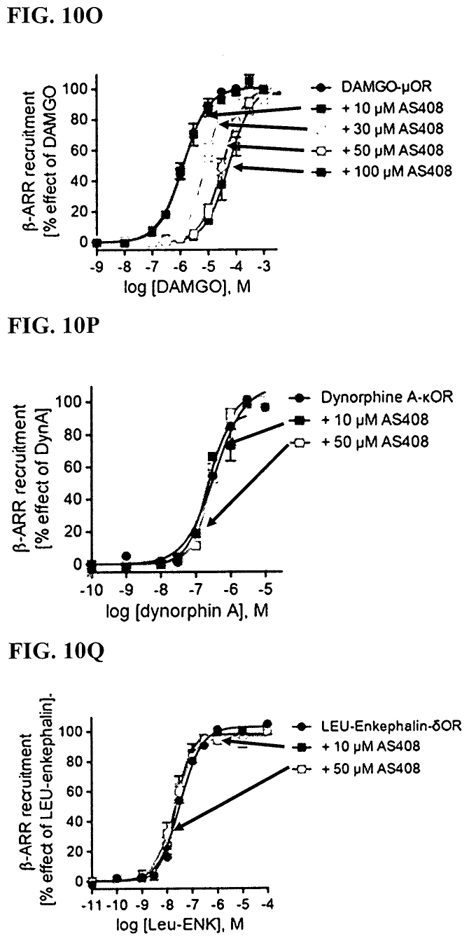

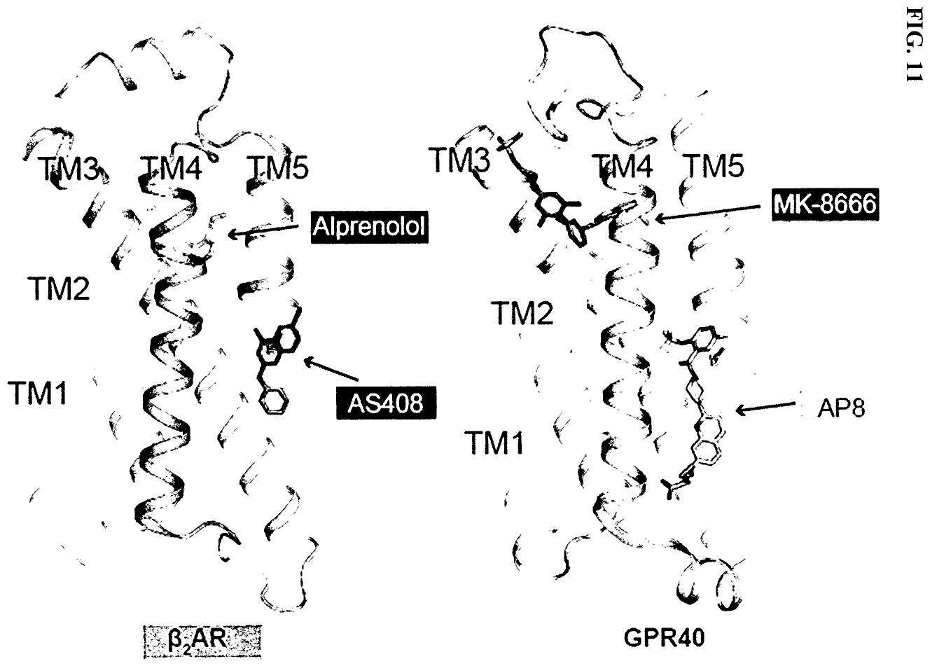

We examined the selectivity of AS408 by performing arrestin recruitment assays on 12 family A GPCRs (FIG. 10A-10Q). FIG. 10A shows the sequence alignments for the 12 GPCRs. The β1AR is the only other receptor that has E at position 3.41 and differs from the β₂AR only in one amino acid: V^{3.48} in β₂AR and L^{3.48} in β1AR. This small conservative difference leads to a small reduction in the potency of AS408 at the β1AR. AS408 was a weak NAM at the al AR, but had no allosteric activity in the assay used at any of the other GPCRs tested.

Mutations of E122 in the AS408 Binding Pocket

The location of the AS408 binding pocket is of interest given the recent report of a positive allosteric modulator of GPR40 binding to the membrane facing surface of TMs 2, 3, 4 and 5 (6) (FIG. 11), and previous mutagenesis studies revealing that several mutations of E122 lead to enhanced β₂AR expression and thermostability (7). According to our structure, ionic interactions between AS408 in its protonated form and E122^{3.41} are important. To further characterize the role of E122^{3.41} in AS408 binding, we examined the effect of mutating E122^{3.41} to leucine, glutamine and arginine on agonist, antagonist and inverse agonist binding affinity, and on arrestin recruitment and G protein activation. E122Q and E122L expressed at levels comparable to the wild type β₂AR, while expression of E122R was greatly reduced. The effect of AS408 on agonist binding affinity for all of the mutants was reduced relative to the wild-type receptor, with E122L being most similar to wild type for binding to epinephrine. Both E122Q and E122L exhibited substantial reduction in the allosteric response to AS408 in the arrestin recruitment assay, [³⁵S]GTPγS binding and cAMP accumulation. We were unable to detect any agonist stimulated arrestin recruitment and only weak agonist-stimulated [³⁵S]GTPγS binding and cAMP accumulation for E122R. When we examined basal cAMP in cells expressing different levels of E122R, we were able to observe high levels of basal activity relative to WT that could not be suppressed by the inverse agonist ICI-118,551.

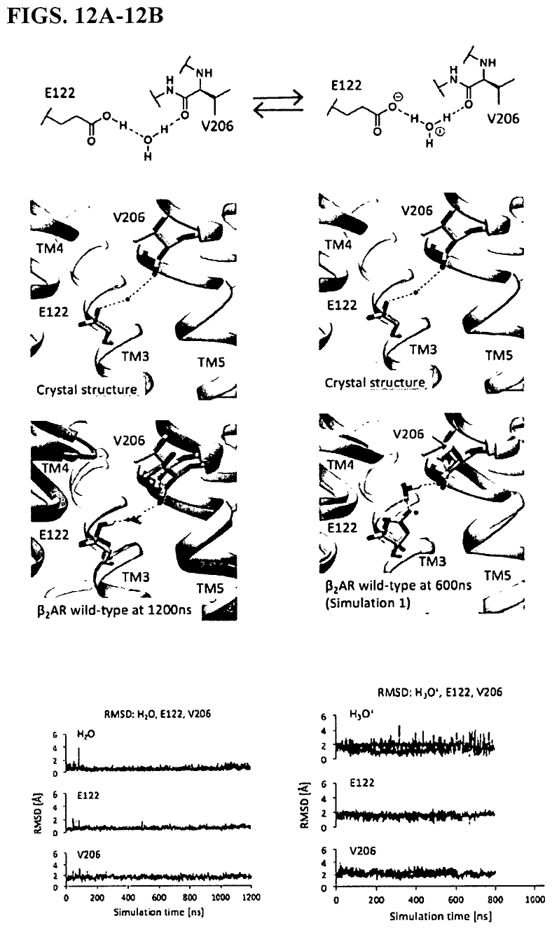

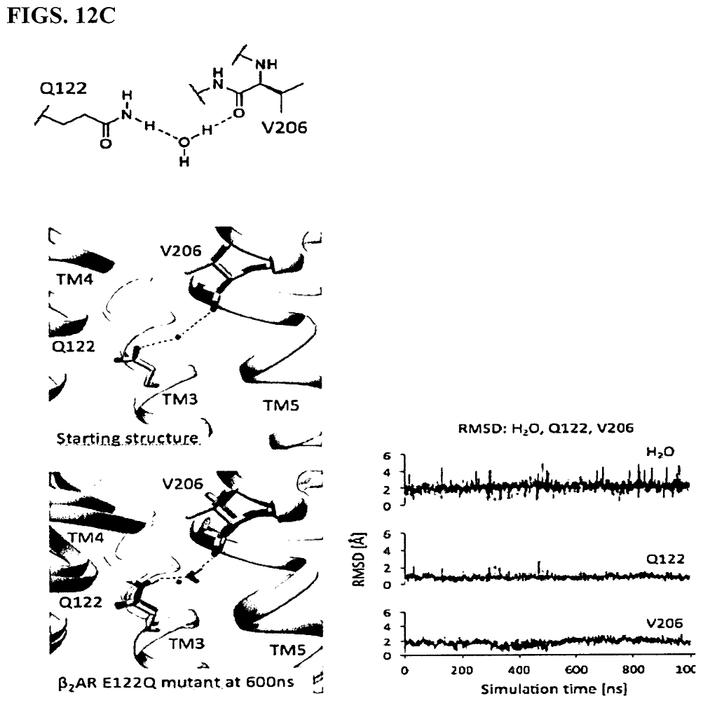

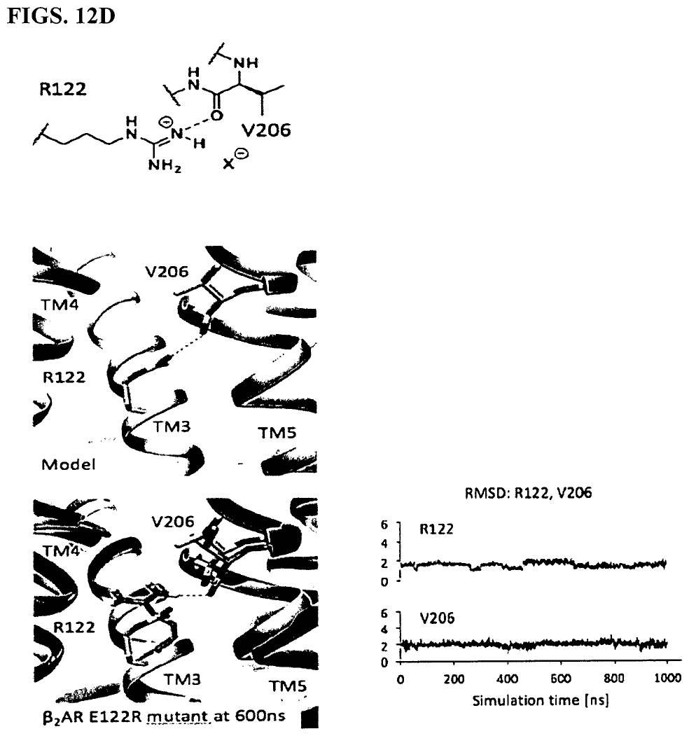

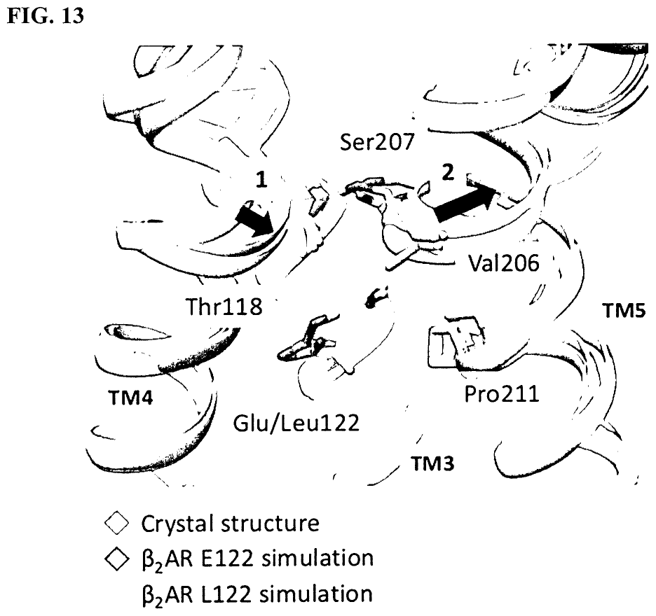

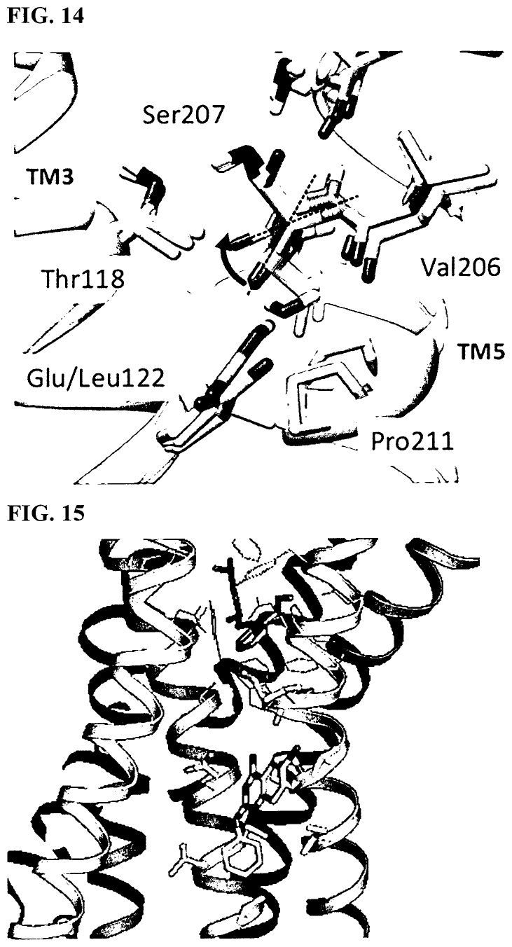

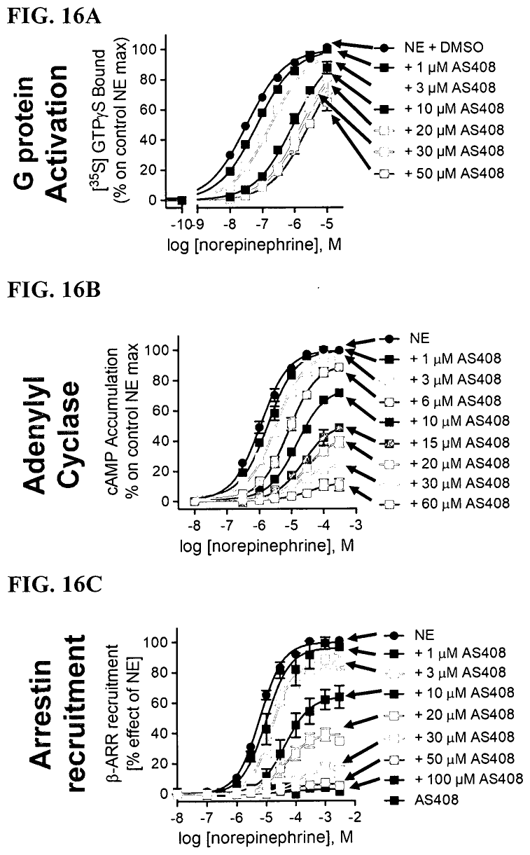

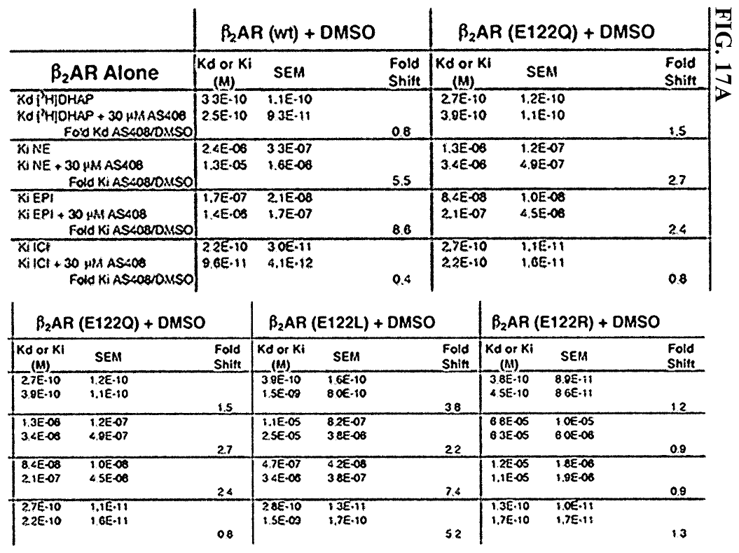

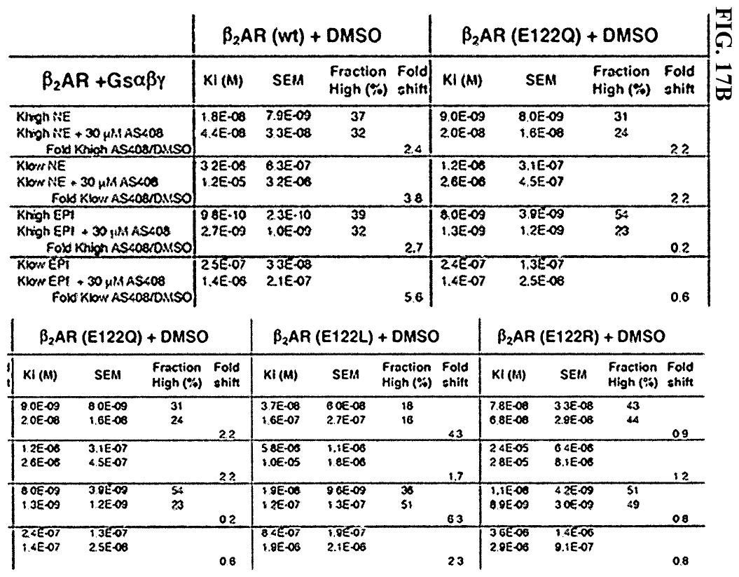

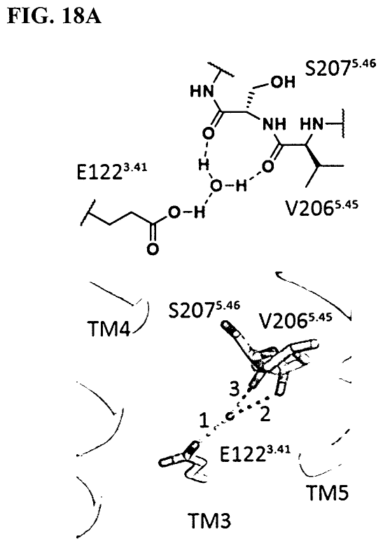

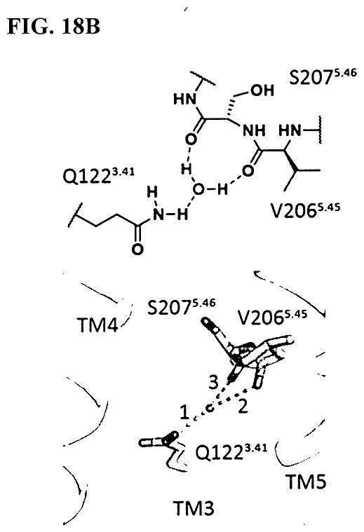

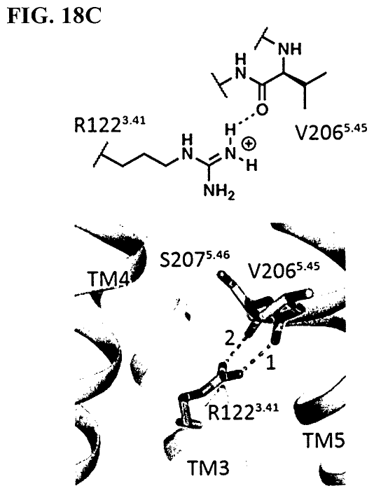

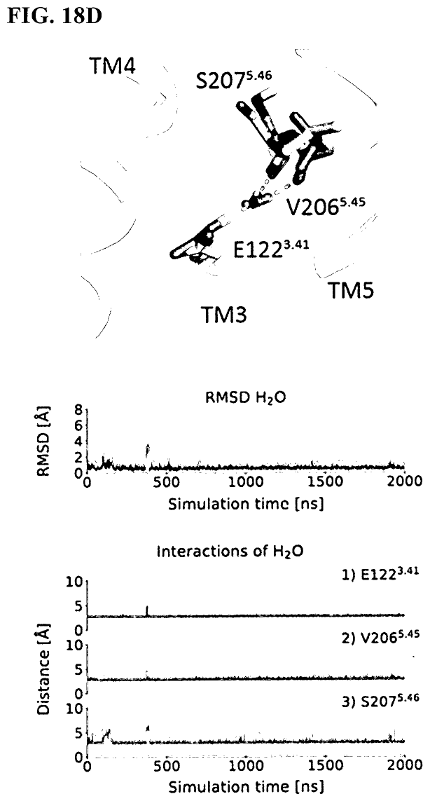

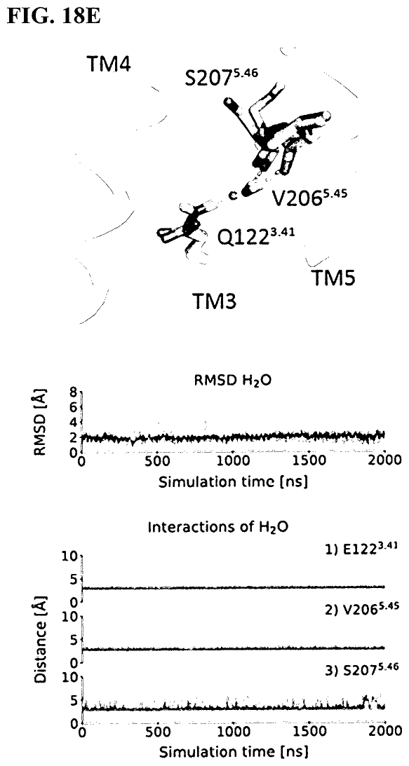

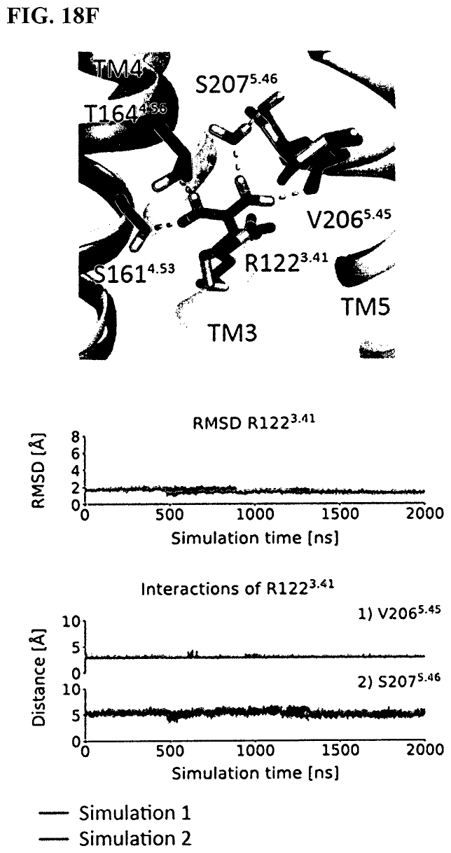

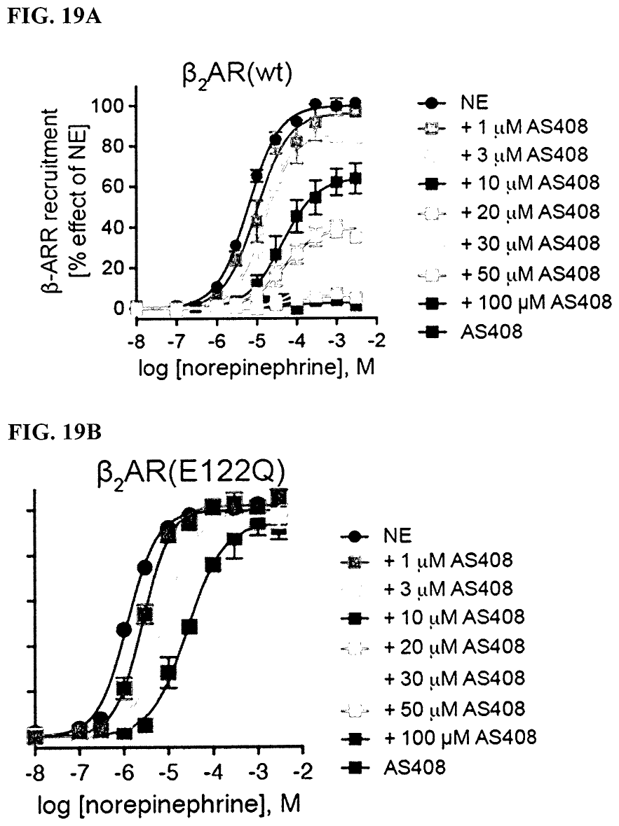

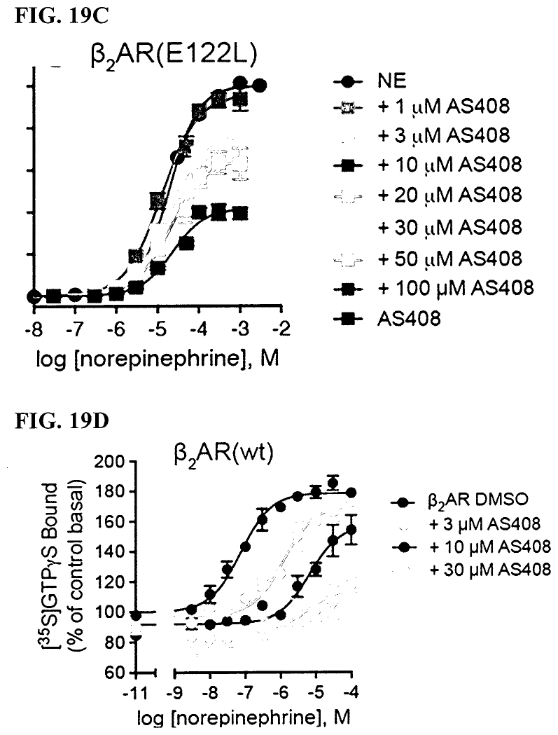

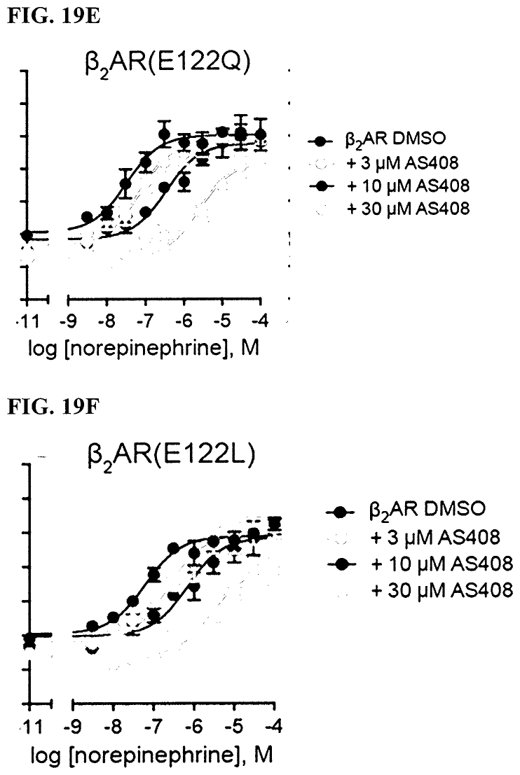

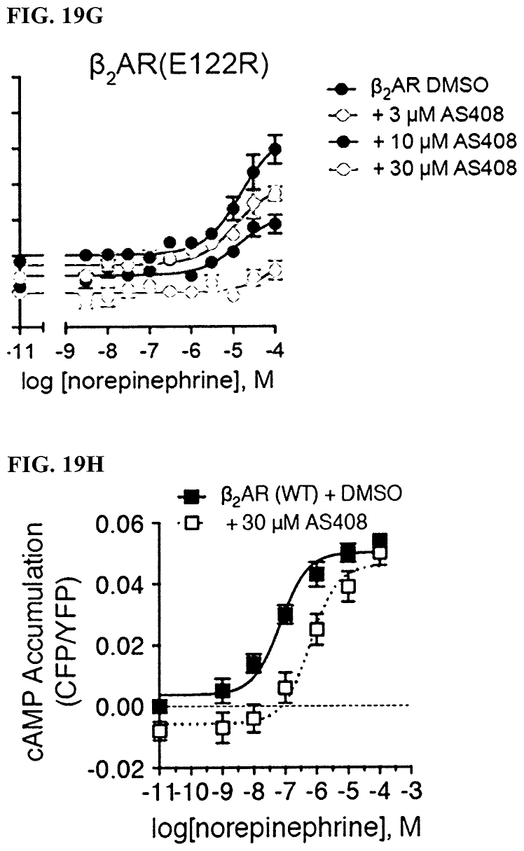

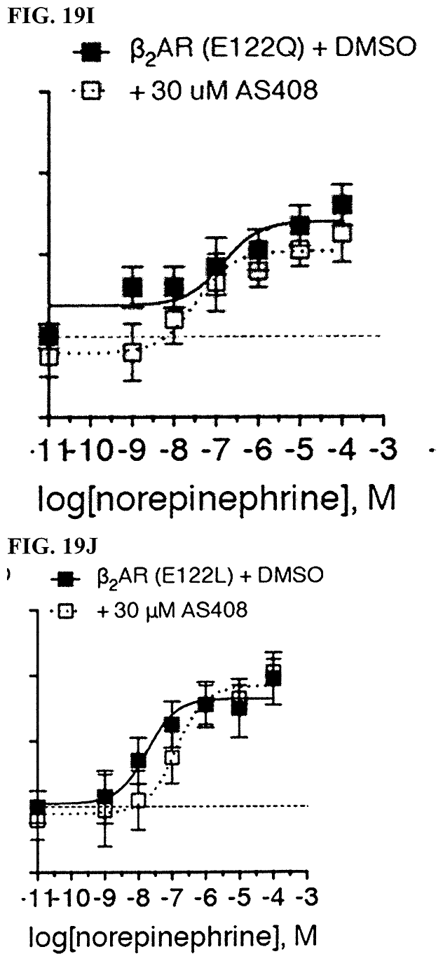

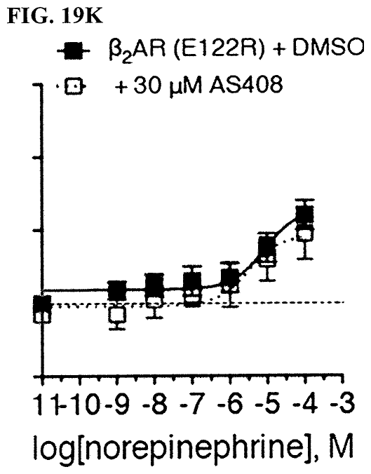

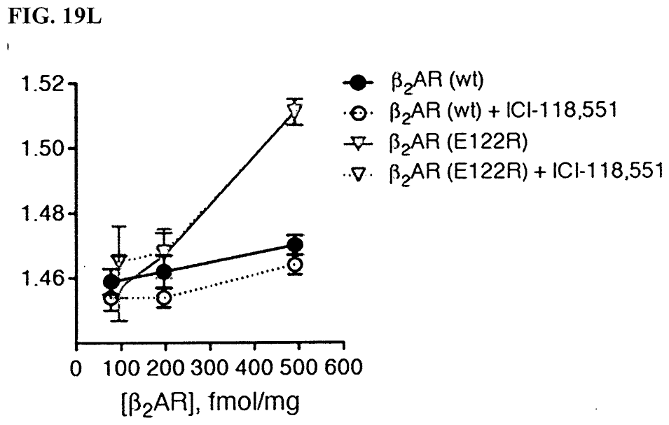

To understand the structural basis for the functional properties of the mutants E122Q and E122R, we performed 16 microseconds all-atom molecular dynamics simulations of the mutants and wild-type (E122^{3.41}) β₂AR. As noted above, there is evidence for a water-mediated hydrogen bond network bridging the carboxylic acid function of E122^{3.41} in TM3 with the backbone carbonyl oxygen of V206^{5.45} and S207^{5.46} in TM5. The E122Q and E122R mutants were modeled based on this structure. For β₂AR wild-type (E122^{3.41}), we considered that either a neutral water molecule or a hydronium cation can mediate this interaction between E122^{3.41} and V206^{5.45} (FIG. 12A and 12B). The mediating water shows a very low RMSD value and the above described interactions were maintained throughout the whole simulation (FIG. 12A). The hydronium is not able to maintain the mediating interactions and the positions of the hydronium and E122^{3.41} substantially deviate from the starting structure (FIG. 11), indicating that the water molecule is unlikely to be protonated in the crystal structure. The water-mediated hydrogen bond network was also observed when performing MD simulations of the β₂AR E122Q mutant. However, the higher RSMD values of the mediating water molecule and the reduced interaction frequency with the carbonyl oxygen of S207^{5.46} (97% at E122 and 85% at Q122) indicate a less stable inactive state, which might explain the higher agonist binding affinity for E122Q. The loss of an allosteric effect of AS408 in the E122Q mutant can be explained by the absence of proton-donating properties of the amide group of glutamine. This results in a less stable hydrogen bond network, because an ionic interaction of E122Q with AS408 in its cationic form is energetically less favorable than the interaction with the carboxylate anion of E122^{3.41} in wild type β₂AR.

The E122R mutant has dramatically reduced agonist-induced arrestin recruitment and G protein activation, but has high basal activity in a cAMP assay. The longer cationic side chain of E122R is expected to directly interact with the V206^{5.45} backbone oxygen stabilizing the inactive receptor conformation (FIG. 12D). In fact, our MD simulations displayed a conformation of E122R that confers a stable ion-dipole interaction with the backbone oxygen of V206^{5.45}. Interestingly, on the course of the simulations the arginine head group loses the contact to the backbone oxygen of S207^{5.46} potentially destabilizing the inactive state. As a consequence, the side chain of S207^{5.46} may contribute to an active-like conformation of TM5 explaining the increased basal activity of the β₂AR E122R mutant and its inability to respond to the inverse agonist ICI-118,551.

In presence of the negative allosteric modulator AS408, the mediating water molecule is displaced by the protonated primary amino group of AS408. Because AS408 is more basic than water, the ionic character of the interaction with the carboxylate anion of E122^{3.41} in wild type β₂AR is substantially higher than in the absence of the modulator. This explains the particular stabilization of the inactive state of the receptor when bound to AS408. The absence of proton-donating properties of the amide group of the β₂AR E122Q mutant results in a less stable hydrogen bond network, because an ionic interaction with AS408 in its cationic form is energetically less favorable. Thus, AS408 exhibits weaker binding and modulation upon mutation by glutamine.

We present the structure of the β₂AR bound to AS408, a newly discovered negative allosteric modulator. The AS408 binding site is composed of lipid bilayer facing residues in TM3 and TM5. The binding pocket includes only one polar amino acid, E122^{3.41}. This site is located adjacent to a conformation hub composed of P211^{5.50}, I121^{3.40} and F282^{6.44}, which undergo packing rearrangements upon activation. The crystal structure together with MD simulations provides insights into the mechanism by which AS408 acts as a NAM for the β₂AR.

Methods

Molecular Dynamics Simulations. Simulations of AS408 at β₂AR were based on the crystal structure described in this manuscript. Coordinates were prepared by removing the T4L fragment and crystal water associated with T4L. The two cholesterol molecules, alprenolol, AS408 a crystal water close to the receptor were retained. UCSF Chimera (8) was used to model missing side-chains. Hydrogens were added and the protein chain termini were capped with acetyl and methylamide. Simulations of β₂AR wild-type (E122), the mutants E122Q and E122R were based on the β₂AR crystal structure and were prepared in the same manner.

Except for the neutral E122 in the β₂AR wild type (E122) simulation with a mediating water molecule, all titratable residues were left in their dominant protonation state at pH 7.0.

Alprenolol and AS408 were protonated at the secondary amine and the primary amino group, respectively. The protein structures were then align to Orientation of Proteins in Membranes (OPM) (9) structure of β₂AR (PDB entry 4GBR). Each complex was inserted into a pre-equilibrated membrane of dioleoyl-phosphatidylcholine (DOPC) lipids by means of the GROMACS tool g_membed (10). Subsequently, sodium and chlorine ions were added to give a neutral system with 0.15M NaCl. The system dimensions were roughly 80×80×100 Å³, containing 156 lipids 58 sodium ions, 66 chlorine ions (67 in E122R system) and about 13.000 water molecules.

Parameter topology and coordinate files were build up using the tleap module of AMBER16 (11) and subsequently converted into GROMACS input files. For all simulations, the general AMBER force field (GAFF) (12) was used for alprenolol and cholesterol, the lipid 14 force field (13) for DOPC molecules and ff14SB (14) for the protein residues. The SPC/E water model (75) was applied. Parameters for ligands were assigned using antechamber¹¹. Structures of the ligands were optimized by means of Gaussian 09 (62) at the B3LYP/6-31G(d) level and charges were calculated at HF/6-31G(d) level and the RESP procedure according to literature (77). A formal charge of +1 was defined for alprenolol and AS408. For the hydronium molecule we used the parameters from M. Baaden et al. (18).

Simulations were performed using GROMACS 5.1.3 (19,20). The simulation systems were energy minimized and equilibrated in the NVT ensemble at 310K for 1 ns followed by the NPT ensemble for Ins with harmonic restraints of 10.0 kcal·mol⁻¹ on protein and ligands. In the NVT ensemble the V-rescale thermostat was used. In the NPT ensemble the Berendsen barostat and a surface tension of 22 dyn·cm⁻¹ and a compressibility of 4.5×10⁻⁵ bar⁻¹ was applied. The system was further equilibrated for 2 ns with restraints on protein backbone and ligands and additional 16 ns without restraints. Multiple simulations were started from the final snapshot of the equilibration resulting in productive molecular dynamics simulation runs of 2-4 μs.

Simulations were performed using periodic boundary conditions and time step of 2 fs with bonds involving hydrogen constrained using LINCS. Long-range electrostatic interactions were computed using particle mesh Ewald (PME) method with interpolation of order 4 and FFT grid spacing of 1.6 Å. Non-bonded interactions were cut off at 12.0 Å.

The analysis of the trajectories was performed using the CPPTRAJ module of AMBER16 and visualization was performed using the UCSF Chimera package 1.11 (5) or PyMOL Molecular Graphics System, Version 2.1.1 (Schrödinger, LLC). Distance and rmsd were plotted using Matplotlib, Version 2.2.2.

β-Arrestin-2 Recruitment Assay. Determination of β-arrestin-2 recruitment was performed applying the PathHunter assay (DiscoverX, Birmingham, U.K.) which is based on fragment complementation of β-galactosidase in HEK293 cells stably expressing (EA)-β-arrestin-2 and being transiently transfected with a receptor tagged to the PK fragment. In general, cells were transfected employing Mirus TransIT-293 (peqlab, Erlangen, Germany) and incubated in DMEM/F12 medium (Life Technologies, Darmstadt, Germany) at 37° C. and 5% of CO₂. After 24 hrs cells were detached with Versene (Life Technologies) and transferred into 384-well plates (white plate, transparent bottom, Greiner Bio-One, Frickenhausen, Germany) at a density of 5000 cells/well using the medium CP4 Reagent (DiscoverX). After further 24 hrs of incubation test compounds dissolved in PBS were added to the cells at a final volume of 25 μL and incubated at 37° C. for a distinct time which was optimized for each receptor (details are summarized in the supporting information). Determination of β-arrestin-2 recruitment was started by adding detection mix, incubation at room temperature for 60 min and measuring chemoluminescence with a Clariostar plate reader (BMG, Ortenberg, Germany). For measuring allosteric effects the modulator was preincubated with the cells at a distinct concentration for 30 min followed by the addition of reference agonist. Data analysis of functional experiments were performed by normalizing the raw data relative to basal activity (0%) and the maximum effect of the reference agonist (100%). Normalized curves from three to seven individual experiments each done as duplicate were analyzed by non-linear regression applying the algorithms in Prism 6.0 (GraphPad, San Diego, Calif.) to get dose-response curves representing average EC₅₀ and Eₘₐₓ value.

Protein expression and purification. A previously reported β₂AR-T4L (27) construct was cloned into pFastbac vector and fusion protein was expressed in sf9 cells using the Bac-to-Bac baculovirus expression system. Cells were infected with high dose baculovirus at density of around 4×10⁶ cells per milliliter and harvested at 48 hours after infection. 10 μM alprenolol was added to enhance expression. β₂AR-T4L was extracted from cell membrane with DDM buffer and was purified in the same way as previously described (22), using a first M1 Flag affinity column, followed by alprenolol-Sepharose chromatography (22) and a second M1-Flag affinity column. 100 μM alprenolol was added to the all the buffers used in the second M1 chromatography, during which detergent was exchanged from 0.1% DDM to 0.01% MNG. The purified β₂AR-T4L was dialyzed against dialysis buffer (20 mM HEPES, pH7.5, 100 mM NaCl, 0.003% MNG, 0.0003% CHS, 100 μM alprenolol) overnight at 4° C. PNGase F was added to remove N-linked sugars. The protein was concentrated to ˜50 mg/mL with a 50 KDa cutoff Amicon centrifugal filters (Millipore). If not used immediately, the protein was flash frozen with liquid nitrogen and stored at −80° C.

Crystallization. Lipidic cubic phase (LCP) crystallizations of β₂AR-T4L in complex with alprenolol and AS408 were performed using a LCP crystallization robot (Gryphon, Art Robbins Instruments). In brief, protein solution was mixed with 9:1 (w/w) monoolein:cholesterol (Sigma) with protein to lipid ratio of 2:3 (w/w) and reconstituted into LCP using two-syringe method (23). 96-well glass sandwich plates were filled with 30 nL LCP overlaid with 1 μL precipitant solution and incubated at 20° C. The best crystals were grown in conditions containing 0.1 M Tris-HCl, pH 8.0, 30%-40% PEG400, 300 mM-400 mM sodium formate, 6% 1,4-butanediol, 1 mM alprenolol, 1 mM AS408 and 1% DMSO.

Data collection and structure determination. X-ray diffraction data were collected at beamline BL32XU at Spring-8, Japan. Typically wedges of 5-10° were collected for each crystal using a 10 μm×10 μm beam. Diffraction data were processed using XDS (24). A full 3.1 Å dataset was obtained by merging data from 36 crystals. Crystal structure was solved by molecular replacement using high-resolution β₂AR-T4L structure (PDB, 2RH1) as searching model. The allosteric modulator AS408 was manually fit into the Fo-Fc electron density maps in coot (25). Structure refinement was performed with phenix.refine (26). The final model was validated using Molprobity (27). Data processing statistics and structural refinement statistics were shown in table 1. Structure figures were prepared using Pymol (The PyMOL Molecular Graphics System, Schrödinger, LLC.).

Radioligand binding assay. To determine the allosteric effect of AS408 on orthosteric ligand binding membranes prepared from Sf9 cells expressing β₂AR or mutants alone or co-infected with GsαPγ, were tested for their capacity to modulate [³H]DHAP binding, as described. Typically, β₂AR membranes (1-10 μg) were incubated for 3 h in binding buffer (20 mM HEPES, pH 7.4, 100 mM NaCl, 1 mM ascorbic acid) with 0.2 nM [³H]DHAP along with varying concentrations of orthosteric ligand in the absence or presence of varying concentration of AS408 (or with 50 μM propranolol to determine non-specific binding). To test the capacity of AS408 to accelerate the dissociation of agonist [³H]formoterol, β₂AR membranes (with GsαPγ) were preincubated in binding buffer with 2 nM [³H]formoterol, 10 mM MgCl₂, 10 μM GTPγS for 60 min at room temperature. Dissociation was initiated by dilution with the assay buffer containing 200 μM propranolol in the absence or presence of AS408 marking t=0 min. Samples were taken at varying time points, filtered and washed as described below to remove free [³H]formoterol. For [³H]DHAP saturation isotherms of β₂AR and mutants, membranes were incubated with varying concentrations of [³H]DHAP and filtered as described below. Samples were subject to rapid filtration through GF/B membranes and rinsed with ice cold binding buffer to remove free [³H]probe. Filter plates were dried before adding Microscint 0 and counting bound [³H]probe using a Packard TopCount. All data were analyzed using Graphpad (Prism, San Diego Calif.).

Purified β₂AR in DDM or phospholipid. Purified β₂AR was reconstituted into high density lipoprotein particles comprised of apolipoprotein A1 and a 3:2 (mol:mol) mixture of POPC:POPG lipid or a 3:2:1.25 (mol:mol:mol) mixture of POPC:POPG:cholesterol lipid (28). Another sample was prepared by incubating M1-FLAG affinity resin (SIGMA) with purified β₂AR in DDM buffer. In total 3 samples were prepared, which were β₂AR in POPC/POPG HDL particles, β₂AR in POPC/POPG/cholesterol HDL particles and M1 resin bound β₂AR in DDM buffer. Radioligand binding assays were performed to all these three samples. Binding reactions were 500 μL in volume, containing 100 fmol functional receptor, 2 nM ³H dihydroalprenolol (³H-DHA), 100 mM NaCl, 20 mM Tris pH 7.5, 1 mM Ca²⁺, 0.2% bovine serum albumin, and various concentration of AS408 as indicated. 0.02% DDM was added in reactions for M1 resin bound β₂AR samples. Reactions were mixed and incubated for 2 hours at room temperature before harvested with a Brandel 48-well harvester by filtering onto a filter paper pre-treated with 0.3% polyethylenimine. Radioactivity was measured by liquid scintillation counting. All experiments were triplicated and presented as means±standard error of mean.

[³⁵S]GTPγS binding assay. Membranes were prepared from High Five™ (Invitrogen) or S/9 cells expressing β₂AR or mutants and GsαPγ. Typically, membranes (2-5 μg) were pretreated with GDP (final assay concentration of 10 μM) in assay buffer (20 mM HEPES, pH 7.4, 100 mM NaCl, 10 mM MgCl₂, and 1 mM ascorbic acid) and different concentrations of AS408 for 20 min at room temperature before adding [³⁵S]GTPγS (for a final concentration of 0.1 nM) with a range of concentrations of agonist (epinephrine or norepinephrine). For most cases the assays were incubated at room temperature for a period of 1 h before stopping by rapid filtration through GF/B membranes and washing with ice-cold assay buffer. To determine the Kb for AS408 on β₂AR and mutants, assay times were reduced to 10 min at 30° C., in order to avoid saturating [³⁵S]GTPγS binding to Gs. Assays were performed in a 96-well microplate format and radioactivity was measured using a TopCount (Packard).

cAMP accumulation assays. Intact cell cAMP accumulation was measured using the FRET-epac sensor in stable HEK293-Epac cells endogenously expressing β₂AR or in CHO cells co-transfected with Epac and β₂AR or the mutants. Cells were harvested with lifting buffer (20 mM HEPES, pH 7.4, 150 mM NaCl and 0.68 mM EDTA), centrifuged and resuspended in HBSS-HEPES (Hank's Balanced Salt Solution plus 20 mM HEPES, pH 7.4) containing 0-150 μM modulator or vehicle (for a final assay concentration of 0-100 μM). This cell suspension (100 μL) was pipetted into the wells of a 96 well plate (black with clear bottom). After 20 min in the dark at 37° C., 50 μL of HBSS-HEPES buffer at 37° C. containing 1B MX (1 mM final), ascorbic acid (1 mM final), and norepinephrine or epinephrine (0-100 μM final) was added. The CFP/YFP ratio of the Epac-cAMP FRET sensor was immediately measured for 15 min using wavelengths of 435 nm for excitation with 485 nm and 530 nm for emission using a SpectraMax M5 (Molecular Devices). The CFP/YFP ratio area under the curve for 10 min was used to determine maximal agonist-stimulated cAMP accumulation and EC₅₀ using GraphPad Prism 6.0 (San Diego Calif.).

β-Arrestin-2 Recruitment Assay. β-Arrestin-2 recruitment was performed applying the fragment complementation assay PathHunter (DiscoverX) with HEK293 cells stably expressing (EA)-β-arrestin-2. The appropriate receptor was transiently transfected when using a specific pCMV vectors with the PK-tag located at different distances downstream of the C-terminus of the inserted receptor (PK1, PK2 and PK-ARMS2, purchased from DiscoverX, Birmingham, UK). ADRB2-PK encoding the human β₂AR was purchased from DiscoverX, while all other vectors with related GPCRs were engineered by inserting the DNA of the appropriate receptor in frame into the different PK constructs further excluding the stop codon. Mutants of the β₂AR were done applying polymerase chain reaction with appropriate primers. Table 2 shows an overview of all applied constructs, the best working PK-tag, the appropriate reference agonist and the optimized time of incubation,

TABLE 2
Vectors and corresponding experimental details
applied for β-arrestin-2 recruitment assays.

| | | | time of |
|---|---|---|---|
| Receptor | vector | agonist | incubation [min] |
| β₂AR | ADRB2-PK1 | norepinephrine | 90 |
| β₂AR E122L | ADRB2-E122L- | norepinephrine | 90 |
| | PK1 | | |
| β₂AR E122Q | ADRB2-E122Q- | norepinephrine | 90 |
| | PK1 | | |
| β₁AR | ADRB1-PK1 | norepinephrine | 90 |
| α_{1A}AR | ADRA1A-PK1 | norepinephrine | 300 |
| α_{2A}AR | ADRA2A-PK- | norepinephrine | 300 |
| | ARMS2 | | |
| 5-HT_{1A}R | 5HT1AR-PK- | serotonin | 180 |
| | ARMS2 | | |
| M₂AChR | M2R-PK- | carbachol | 150 |
| | ARMS2 | | |
| D_{2long}DR | D2LR-PK- | quinpirole | 300 |
| | ARMS2 | | |
| PAR2 | PAR2R-PK1 | f-LIGKV-NH₂ | 90 |
| NTS1R | NTS1R-PK1 | NT8-13 | 90 |
| μOR | MOR-PK1 | DAMGO | 90 |
| κOR | KOR-PK1 | dynorphine A | 300 |
| δOR | DOR-PK2 | LEU- | 90 |
| | | enkephalin | |

### Example 2: Synthesis of AS408 and Analogs

General. All chemicals and solvents were purchased from Sigma Aldrich, Acros, Alfa Aesar, or Activate Scientific and were used without additional purification. Anhydrous solvents were of the highest commercially available grade and were stored over molecular sieves under a nitrogen atmosphere. Flash chromatography was performed on Merck silica gel 60 (40-63 μm) as stationary phase under positive pressure of dry nitrogen gas. TLC analyses were performed using Merck 60 F254 aluminum plates in combination with UV detection (254 nm). FIR-MS was run on a AB Sciex Triple TOF660 Sciex, source type ESI, or on a Bruker maXis MS in the laboratory of the Chair of Organic Chemistry, Friedrich Alexander University Erlangen-Nuernberg, or on a Bruker maXis MS in the laboratory of the Chair of Bioinorganic Chemistry, Friedrich Alexander University Erlangen-Nuernberg. Mass detection was conducted with a Bruker Esquire 2000 ion trap mass spectrometer using APCI or ESI ionization source or with Bruker amaZon SL mass spectrometer in combination with a Agilent 1100 or Dionex Ultimate 3000 UHPLC system; respectively. Analytical HPLC was conducted on an Agilent 1200 HPLC system employing a DAD detector and a ZORBAX ECLIPSE XDB-C8 (4.6×150 mm, 5 μm) column with the following binary solvent systems: System 1: eluent, methanol/0.1% aq formic acid, 10% methanol for 3 min, to 100% in 15 min, 100% for 6 min, to 10% in 3 min, then 10% for 3 min, flow rate 0.5 mL/min, λ=210 or 254 nm; System 2: CH₃CN/0.1% aq formic acid, 10% CH₃CN for 3 min, to 100% in 15 min, 100% for 6 min, to 10% in 3 min, then 10% for 3 min, flow rate 0.5 mL/min, λ=210 or 254 nm. Preparative HPLC was performed on an Agilent 1100 Preparative Series, using a ZORBAX ECLIPSE XDB-C8 PrepHT (21.5×150 mm, 5 μm, flow rate 10 mL/min) column with the solvent systems indicated. ¹H, and ¹³C and DEPTQ NMR spectra were recorded on a Bruker Avance 360, Avance 400 or a Bruker Avance 600 FT-NMR-Spectrometer. Chemical shifts were calculated as ppm relative to TMS (¹H) or solvent signal (¹³C) as internal standards.

Chemical synthesis of AS408. 6-Bromo-2,4-dichloroquinazoline (234 mg, 1 eq, 0.85 mmol) was dissolved in dry THF (2 mL). Aqueous cone, ammonia (1.5 mL) was added and the reaction mixture was stirred for 2 h at an ambient temperature. After removal of THF under reduced pressure, the aqueous solution was lyophilized, and the crude material was purified by silica gel chromatography (CH₂Cl₂/MeOH, 30:1 v/v) to afford 6-bromo-2-chloroquinazolin-4-amine (185 mg, 85%) as a light beige solid; ¹H NMR (600 MHz, DMSO-d₆) δ 8.54 (d, J=2.1 Hz, 1H), 8.43 (br s, 2H), 7.93 (dd, J=8.9, 2.2 Hz, 1H), 7.56 (d, J=8.9 Hz, 1H); ¹³C NMR (100 MHz, DMSO-d₆) δ 163.9, 158.1, 148.2, 137.3, 126.6, 123.7, 121.1, 114.5; ESI-MS m/z 257.9 [M+H]⁺. Aniline (28 μL, 4 eq, 0.31 mmol) was added to a solution of 6-bromo-2-chloroquinazolin-4-amine (19.8 mg, 1 eq, 0.08 mmol) in anhydrous ethanol (˜2 mL) in a pressure tube and the reaction mixture was stirred at 80° C. for 16 h. The solvent was evaporated, and the crude material was treated with saturated and aqueous NaHCO₃ and, subsequently, extracted three times with CH₂Cl₂. The combined organic layers were dried (MgSO₄) and the solvent was evaporated. The crude material was purified by preparative HPLC (acetonitrile in 0.1% aq. HCOOH, 5% to 95%) to yield 6-bromo-N²-phenylquinazoline-2,4-diamine (AS408) as a light beige solid (25.0 mg, 90%); ³H NMR (600 MHz, DMSO-d₆) δ 9.05 (s, 1H), 8.35 (d, J=2.2 Hz, 1H), 8.14 (br s, 1H), 7.95-7.86 (m, 2H), 7.69 (dd, J=8.9, 2.2 Hz, 1H), 7.57 (br s, 2H), 7.34 (d, J=8.9 Hz, 1H), 7.30-7.20 (m, 2H), 6.97-6.83 (m, 1H); ¹³C NMR (150 MHz, DMSO-di) δ 161.7, 157.9, 150.9, 141.6, 136.0, 128.7 (2C), 127.9, 126.4, 121.1, 119.2 (2C), 113.3, 113.0; ESI-MS m/z 315.0 [M+H]⁺; HRMS-ESI (m/z) [M+H]⁺: calcd. for C₁₄H₁₂BrN₄: 315.0240, found: 315.0238; HPLC: System 1: t_{R}=16.0 min, purity 97%, System 2: t_{R}=13.7 min, purity 99%.

Scheme 1 below shows a) urea, 150° C., 16 h, b) POCl₃, PhN(Me)₂, 120° C., 16 h, c) NH₄OH, THF, 2 h, d) aniline, EtOH, 80° C., 16 h, e) ST239, PhB(OH)₂, Na₂CO₃, Pd(dppf)Cl₂, dioxane/H₂O, 80° C., 3 h.

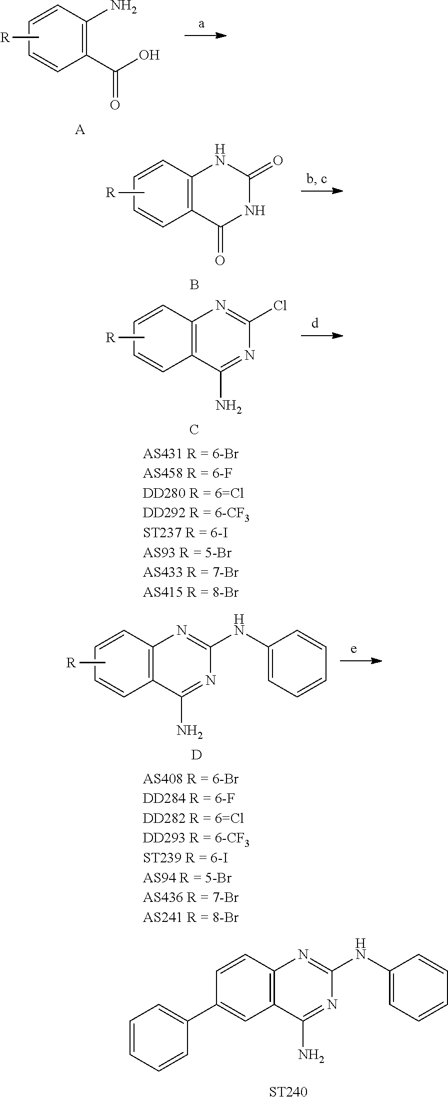

The library of different phenylquinazoline-2,4-diamines based on the BRAC1 (Scheme 2) substructure was easily accessible by utilizing a modified, previous described procedure (7,2), starting with a cyclization reaction of commercially available anthranilic acids A with urea to the quinazoline-2,4(1H,3H)-diones B. Chlorination to the 2,4-dichloroquinazolines was conducted with phosphoryl oxychloride, followed by a selective substitution reaction in 4-position in aqueous ammonia to the 2-chloroquinazolin-4-amines C. Refluxing with the aniline in ethanol resulted in the final phenylquinazoline-2,4-diamines D. The biphenyl derivative ST240 was achieved via a Suzuki coupling reaction of the 6-iodo-N²-phenylquinazoline-2,4-diamine ST239 with phenylboronoic acid.

Scheme 2 below shows a) aniline, EtOH, 80° C., 6 h.

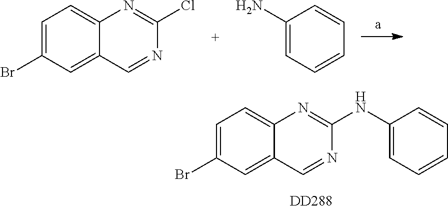

The desamino quinazoline derivative of AS408 was obtained under similar conditions (Scheme 2) starting with 6-bromo-2-chloroquinazoline.

General procedure for the synthesis of the 2-chloroquinazolin-4-amines C. According to a modified, previous described procedure (Keov, P. et al, Neuropharmacology 2011, 60, 24-35), the anthranilic acid A (1 eq.) was added portion wise to melted urea (10 eq.) and the mixture was stirred at 150° C. for 16 h. After cooling to room temperature, water was added and the mixture was sonicated for 30 min to get a finely dispersed precipitate, which was collected by suction filtration, washed several times with water and dried in vacuo. The obtained quinazoline-2,4(1H,3H)-dione B (1 eq.) was suspended in POCl₃ (˜0.5 mL/mmol) at room temperature, and N,N-dimethylaniline (cat. amounts, 2-3 drops) was added. After the reaction mixture was stirred at 120° C. for 16 h, it was cooled to room temperature and poured carefully on ice. The formed precipitate was collected by suction filtration, washed several times with water and was directly dissolved in THF (2-3 mL/mmol). Aqueous ammonia (25%, 1-2 mL/mmol) was added and the reaction mixture was stirred for 2 h at room temperature. After removal of THF under reduced pressure the aqueous solution was lyophilized to obtain the 2-chloroquinazolin-4-amine C, which was used in the next step without further purification, otherwise it is indicated below.

Compounds were prepared following general procedure for the synthesis of the 2-chloroquinazolin-4-amines C.

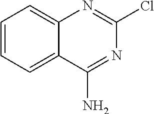 [SMILES: Nc1nc(Cl)nc2ccccc12]

2-Chloroquinazolin-4-amine (AS076). Starting with quinazoline-2,4(1H,3H)-dione and purification of the crude material by silica gel chromatography (CH₂Cl₂/MeOH, 30:1 v/v) resulted in AS076 (670 mg, 3.84 mol, 62%, over 2 steps) as a light beige solid; ³H NMR (360 MHz, DMSO-d₆) δ 8.31 (br s, 2H), 8.23 (dd, J=8.2, 0.8 Hz, 1H), 7.80 (ddd, J=8.3, 7.0, 1.3 Hz, 1H), 7.61 (dd, J=8.3, 0.6 Hz, 1H), 7.52 (ddd, J=8.2, 7.0, 1.2 Hz, 1H); ¹³C NMR (90 MHz, DMSO-d₆) δ 164.0, 157.4, 151.2, 134.3, 126.9, 126.3, 124.3, 113.4; ESI-MS m/z 179.9 [M+H]⁺.

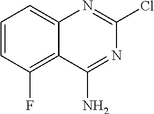 [SMILES: Nc1nc(Cl)nc2cccc(F)c12]

2-Chloro-5-fluoroquinazolin-4-amine×HCl (AS201). Starting with 2-amino-6-fluorobenzoic acid resulted in AS201 (53 mg, 0.23 mmol, 15% over 3 steps) as a yellow solid, which was used in the next step without further purification; ³H NMR (400 MHz, DMSO-d₆) δ 8.64 (br s, 2H), 7.84-7.76 (m, 1H), 7.45 (d, J=8.0 Hz, 1H), 7.32 (ddd, 7=11.7, 8.0, 0.8 Hz, 1H); ¹³C NMR (100 MHz, DMSO-d₆) δ 161.1 (d, J_{CF}=3.7 Hz), 158.6 (d, J_{CF}=254.6 Hz), 157.4, 152.9; 134.5 (d, J_{CF}=10.8 Hz), 122.7 (d, J_{CF}=3.7 Hz), 111.4 (d, J_{CF}=21.8 Hz), 103.3 (d, J_{CF}=11.8 Hz); ESI-MS m/z 197.8 [M+H]⁺.

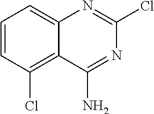 [SMILES: Nc1nc(Cl)nc2cccc(Cl)c12]

2,5-Dichloroquinazolin-4-amine×HCl (AS097/AW03b/JT20/MM08). Starting with 2-amino-6-chlorobenzoic acid resulted in AS097 (201 mg, 0.80 mmol, 47% over 3 steps) as a light beige solid, which was used in the next step without further purification; ¹H NMR (600 MHz, DMSO-d₆) δ 8.78 (br s, 1H), 8.01 (br s, 1H), 7.81-7.64 (m, 2H), 7.58 (ddd, J=4.5, 3.8, 1.2 Hz, 1H); ¹³C NMR (150 MHz, DMSO-d₆) δ 162.8, 157.3, 154.1, 134.3, 129.9, 128.6, 126.9, 111.3; ESI-MS m/z 213.9 [M+H]⁺.

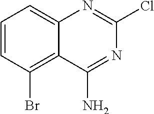 [SMILES: Nc1nc(Cl)nc2cccc(Br)c12]

5-Bromo-2-chloroquinazolin-4-amine×HCl (AS093). Starting with 2-amino-6-bromobenzoic acid resulted in AS093 (60 mg, 0.20 mmol, 56% over 3 steps) as a colorless solid, which was used in the next step without further purification; ³H NMR (600 MHz, DMSO-d₆) δ 8.80 (br s, 1H), 7.93 (br s, 1H), 7.79 (dd, J=7.3, 1.6 Hz, 1H), 7.68-7.61 (m, 2H); ¹³C NMR (150 MHz, DMSO-d₆) δ 162.9, 156.9, 154.1, 134.7, 132.7, 127.6, 118.0, 112.3; ESI-MS m/z 257.7 [M+H]⁺.

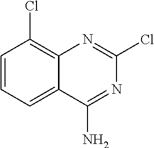 [SMILES: Nc1nc(Cl)nc2c(Cl)cccc12]

2,8-Dichloroquinazolin-4-amine×HCl (AS315). Starting with 2-amino-3-bromobenzoic acid resulted in AS315 (360 mg, 1.44 mmol, 63% over 3 steps) as a brown solid, which was used in the next step without further purification; ³H NMR (400 MHz, DMSO-d₆) δ 8.69 (br s, 1H), 8.54 (br s, 1H), 8.30 (dd, J=8.3, 1.2 Hz, 1H), 7.97 (dd, J=7.7, 1.2 Hz, 1H), 7.52-7.46 (m, 1H); ¹³C NMR (100 MHz, DMSO-d₆) δ 163.8, 158.0, 147.3, 133.8, 129.9, 125.9, 123.3, 114.6; ESI-MS m/z 213.8 [M+H]⁺.

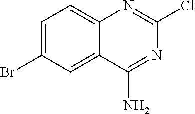 [SMILES: Nc1nc(Cl)nc2ccc(Br)cc12]

6-Bromo-2-chloroquinazolin-4-amine (AS431). Starting with 6-bromo-2,4-dichloroquinazoline and purification of the crude material by silica gel chromatography (CH₂Cl₂/MeOH, 30:1 v/v) resulted in AS431 (185 mg, 0.72 mmol, 85%) as a light beige solid; ¹H NMR (600 MHz, DMSO-d₆) δ 8.54 (d, J=2.1 Hz, 1H), 8.43 (br s, 2H), 7.93 (dd, J=8.9, 2.2 Hz, 1H), 7.56 (d, J=8.9 Hz, 1H); ¹³C NMR (100 MHz, DMSO-d₆) δ 163.9, 158.1, 148.2, 137.3, 126.6, 123.7, 121.1, 114.5; ESI-MS m/z 257.9 [M+H]⁺.

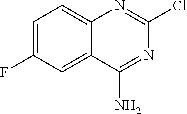 [SMILES: Nc1nc(Cl)nc2ccc(F)cc12]

2-Chloro-6-fluoroquinazolin-4-amine HCl (AS458). Starting with 2-amino-5-fluorobenzoic acid resulted in AS458 (490 mg, 2.09 mmol, 34% over 3 steps) as a light yellow solid, which was used in the next step without further purification; ³H NMR (400 MHz, DMSO-di) δ 8.41 (br s, 2H), 8.17 (dd, J=9.5, 2.5 Hz, 1H), 7.77-7.65 (m, 2H); ¹³C NMR (100 MHz, DMSO-d₆) δ 163.3 (d, 7=3.9 Hz), 159.1 (d, J=244.2 Hz), 156.7, 147.9, 129.3 (d, 7=8.6 Hz), 123.2 (d, 7=24.8 Hz), 113.6 (d, 7=9.1 Hz), 108.5 (d, 7=23.7 Hz); ESI-MS m/z 197.8 [M+H]⁺.

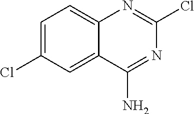 [SMILES: Nc1nc(Cl)nc2ccc(Cl)cc12]

2,6-Dichloroquinazolin-4-amine HCl (DD280). Starting with 2-amino-5-chlorobenzoic acid resulted in DD280 (334 mg, 1.56 mmol, 27% over 3 steps) as a light yellow solid, which was used in the next step without further purification; ³H NMR (400 MHz, DMSO) δ 8.44 (s, 2H), 8.40 (d, 7=2.2 Hz, 1H), 7.83 (dd, 7=8.9, 2.2 Hz, 1H), 7.64 (d, 7=8.9 Hz, 1H); ¹³C NMR (100 MHz, DMSO-76) δ 162.8, 157.4, 149.5, 134.2, 129.9, 128.7, 123.2, 114.0; ESI-MS m/z 213.8 [M+H]⁺.

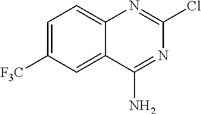 [SMILES: Nc1nc(Cl)nc2ccc(C(F)(F)F)cc12]

2-Chloro-6-(trifluoromethyl)quinazolin-4-amine HCl (DD292). Starting with 2-amino-5-(trifluoromethyl)benzoic acid resulted in DD292 (160 mg, 0.73 mmol, 30% over 3 steps) as an unpure dirty green solid, which was used in the next step without further purification; ESI-MS 247.7 m/z [M+H]⁺.

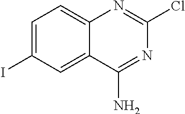 [SMILES: Nc1nc(Cl)nc2ccc(I)cc12]

6-Iodo-2-chloroquinazolin-4-amine HCl (ST237). Starting with 2-amino-5-iodobenzoic acid resulted in ST237 (265 mg, 0.78 mmol, 26% over 3 steps) as a light yellow solid, which was used in the next step without further purification; ¹H NMR (400 MHz, DMSO-d₆) δ 8.67 (d, J=1.9 Hz, 1H), 8.40 (br s, 2H), 8.05 (dd, J=8.8, 1.9 Hz, 1H), 7.39 (d, J=8.8 Hz, 1H); ¹³C/DEPTQ NMR (400 MHz, DMSO-d₆) δ 162.4, 157.3, 150.0, 142.2, 132.4, 128.5, 114.9, 90.6; ESI-MS m/z 305.75 [M+H],

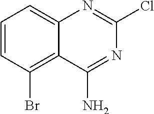 [SMILES: Nc1nc(Cl)nc2cccc(Br)c12]

5-Bromo-2-chloroquinazolin-4-amine HCl (AS93). Starting with 2-amino-6-bromobenzoic acid resulted in AS93 (60 mg, 0.20 mmol, 56% over 3 steps) as a colorless solid, which was used in the next step without further purification; ³H NMR (600 MHz, DMSO-d₆) δ 8.80 (br s, 1H), 7.93 (br s, 1H), 7.79 (dd, J=7.3, 1.6 Hz, 1H), 7.68-7.61 (m, 2H); ¹³C NMR (150 MHz, DMSO-d₆) δ 162.9, 156.9, 154.1, 134.7, 132.7, 127.6, 118.0, 112.3; ESI-MS m/z 257.7 [M+H]⁺.

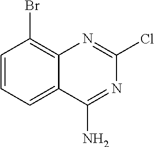 [SMILES: Nc1nc(Cl)nc2c(Br)cccc12]

8-Bromo-2-chloroquinazolin-4-amine (AS415). Starting with 2-amino-3-bromobenzoic acid and purification of the crude material by silica gel chromatography (CH₂Cl₂/MeOH, 30:1 v/v) resulted in AS415 (120 mg, 0.47 mmol, 45% over 3 steps) as a colorless solid; ³H NMR (600 MHz, DMSO-d₆) δ 8.53 (br s, 2H), 8.24 (dd, J=8.2, 1.0 Hz, 1H), 8.14 (dd, J=7.6, 1.0 Hz, 1H), 7.43 (t, 7=7.9 Hz, 1H); ¹³C NMR (150 MHz, DMSO-d₆) δ 163.9, 158.1, 148.2, 137.3, 126.6, 123.7, 121.1, 114.5; ESI-MS m/z 257.8 [M+H]⁺.

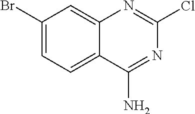 [SMILES: Nc1nc(Cl)nc2cc(Br)ccc12]

7-Bromo-2-chloroquinazolin-4-amine HCl (AS433). Starting with 2-amino-4-bromobenzoic acid resulted in AS433 (780 mg, 2.64 mmol, 65% over 3 steps) as a light beige solid, which was used in the next step without further purification; ¹H NMR (400 MHz, DMSO-d₆) δ 8.62 (br s, 1H), 8.46 (br s, 1H), 8.27 (d, J=8.8 Hz, 1H), 7.83 (d, 7=1.9 Hz, 1H), 7.69 (dd, 7=8.8, 2.0 Hz, 1H); ¹³C NMR (100 MHz, DMSO-d₆) δ 163.5, 158.1, 151.9, 128.9, 128.6, 127.6, 126.2, 112.1; ESI-MS m/z 257.9 [M+H]⁺.

### 2-Chloro-6-isopropylquinazolin-4-amine (ST236)

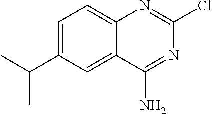

ST236 was prepared as described in the General Procedure for the synthesis of the 2-chloroquinazolin-4-amines C, starting with 2-Amino-5-Isopropylbenzoic acid (250 mg, 1.40 mmol) and urea (838 mg, 13.95 mmol). Yield: 75 mg (34%) brownish solid.

¹H NMR (400 MHz, DMSO-d₆) δ 1.27 (d, 7=6.9 Hz, 6H), 3.01 (sept, 7=6.9 Hz, 1H), 7.54 (d, 7=8.6 Hz, 1H), 7.71 (dd, 7=8.6, 1.9 Hz, 1H), 8.08 (d, 7=1.9 Hz, 1H), 8.24 (br s, 2H); 15 ¹³C/DEPTQ NMR (400 MHz, DMSO-d₆) δ 23.76; 33.55, 112.83, 120.30, 126.37, 133.25, 146.42, 149.38, 156.29, 163.44; ESI-MS m/z 221.8 [M+H]⁺.

General Procedure for the synthesis of the phenylquinazoline-2,4-diamines D.

According to a modified, previous described procedure, aniline (4 eq.) was added to a solution of the 2-chloroquinazolin-4-amine C (1 eq.) in anhydrous ethanol (˜20 mL/mmol) in a pressure tube and the reaction mixture was stirred at 80° C. for 16 h. The solvent was rotary evaporated, and the crude material was treated with saturated, aqueous NaHCO₃ and extracted three times with CH₂Cl₂. The combined organic phases were dried (MgSO₄) and the solvent was rotary evaporated. The obtained residue was purified as indicated below.

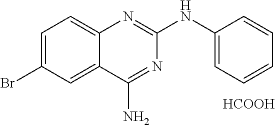 [SMILES: Nc1nc(Nc2ccccc2)nc2ccc(Br)cc12 ; "HCOOH"]

6-Bromo-N²-phenylquinazoline-2,4-diamine×HCOOH (AS408). The crude material was purified by preparative HPLC (acetonitrile in 0.1% aqueous HCOOH, 5% to 95%) to yield AS408 as a light beige solid (25.0 mg, 90%); ³H NMR (600 MHz, DMSO-d₆) δ 9.05 (s, 1H), 8.35 (d, J=2.2 Hz, 1H), 8.14 (br s, 1H), 7.95-7.86 (m, 2H), 7.69 (dd, J=8.9, 2.2 Hz, 1H), 7.57 (br s, 2H), 7.34 (d, J=8.9 Hz, 1H), 7.30-7.20 (m, 2H), 6.97-6.83 (m, 1H); ¹³C NMR (150 MHz, DMSO-d₆) δ 161.7, 157.9, 150.9, 141.6, 136.0, 128.7 (2C), 127.9, 126.4, 121.1, 119.2 (2C), 113.3, 113.0; ESI-MS m/z 315.0 [M+H]⁺; HRMS-ESI (m/z): [M+H]⁺: calcd. for C₁₄H₁₂BrN₄: 315.0240, found: 315.0238; HPLC: System 1: t_{R}=16.0 min, purity 97%, System 2: t_{R}=13.7 min, purity 99%.

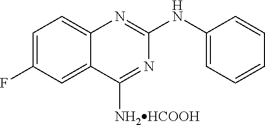

6-Fluoro-N²-phenylquinazoline-2,4-diamine×HCOOH (DD284). The crude material was purified by preparative HPLC (acetonitrile in 0.1% aqueous HCOOH, 15% to 70%) to give DD284 as a white solid (55.0 mg, 85%); ³H NMR (400 MHz, DMSO-d₆) δ 9.00 (s, 1H), 8.18 (br s, 1H), 7.99-7.87 (m, 3H), 7.61-7.40 (m, 4H), 7.25 (t, J=7.9 Hz, 2H), 6.89 (t, J=7.3 Hz, 1H); ¹³C NMR (100 MHz, DMSO-d₆) δ 161.8 (d, J=3.6 Hz), 157.0, 156.7 (d, J=240.4 Hz), 148.4, 141.4, 128.3 (2C), 127.4 (d, 7.9 Hz), 121.9 (d, J=24.4 Hz), 120.5, 118.6 (2C), 111.0 (d, J=8.4 Hz), 108.0 (d, J=22.9 Hz); ESI-MS m/z 255.0 [M+H]⁺; HRMS-ESI (m/z): [M+H]⁺: calcd. for C₁₄H₁₂FN₄: 255.1041, found: 255.1044; HPLC: System 1: t_{R}=14.5 min, purity 98%, System 2: t_{R}=12.6 min, purity 99%.

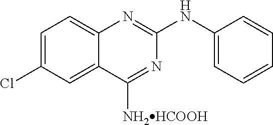

6-Chloro-N²-phenylquinazoline-2,4-diamine×HCOOH (DD282). The crude material was purified by preparative HPLC (acetonitrile in 0.1% aqueous HCOOH, 35% to 90%) to give DD282 as a white solid (63.8 mg, 84%); ³H NMR (400 MHz, DMSO-d₆) δ 9.05 (s, 1H), 8.22 (d, J=2.3 Hz, 1H), 8.17 (br s, 1H), 7.91 (d, J=7.8 Hz, 2H), 7.69-7.49 (br s, 1H), 7.59 (dd, J=8.9, 2.3 Hz, 2H), 7.41 (d, J=8.9 Hz, 1H), 7.25 (t, J=7.9 Hz, 2H), 6.90 (t, J=7.3 Hz, 1H), ¹³C NMR (100 MHz, DMSO-d₆) δ 161.4, 157.5, 150.3, 141.3, 133.1, 128.3 (2C), 127.3, 125.1, 122.9, 120.8, 118.8 (2C), 112.0; ESI-MS m/z 270.9 [M+H]⁺; HRMS-ESI (m/z): [M+H]⁺: calcd. for C₁₄H₁₂ClN₄: 271.0745, found: 271.0747; HPLC: System 1: t_{R}⁼15.3 min, purity 98%, System 2: t_{R}⁼13.1 min, purity 98%.

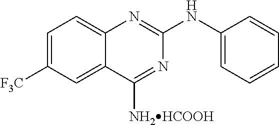

6-Trifluoromethyl-N²-phenylquinazoline-2,4-diamine×HCOOH (DD293). The crude material was purified by preparative HPLC (acetonitrile in 0.1% aqueous HCOOH, 15% to 95%) to give DD293 as a yellowish white solid (9.80 mg, 13%); ³H NMR (600 MHz, DMSO-d₆) δ 9.22 (s, 1H), 8.56 (s, 1H), 7.92 (d, 7=7.7 Hz, 2H), 7.81 (dd, 7=8.8, 1.9 Hz, 1H), 7.77 (br s, 2H), 7.51 (d, J=8.7 Hz, 1H), 7.30-7.22 (m, 2H), 6.95-6.90 (m, 1H). ¹³C NMR (150 MHz, DMSO-d₆) δ 162.3, 158.6, 154.0, 141.0, 128.4 (q, 7=4 Hz), 128.3 (2C), 126.2, 124.6 (q, 7=271.5 Hz), 122.2 (q, 7=4 Hz), 121.1 (q, 7=31.7 Hz), 121.0, 119.1 (2C), 110.4; ESI-MS m/z 304.9 [M+H]⁺; HRMS-ESI (m/z): [M+H]⁺: calcd. for C₁₅H₁₂F₃N₄: 305.1009, found: 305.1010; HPLC: System 1: t_{R}⁼16.1 min, purity 96%, System 2: t_{R}⁼13.7 min, purity 97%.

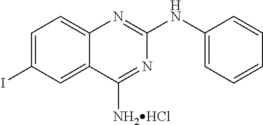

6-Iodo-N²-phenylquinazoline-2,4-diamine×HCl (ST239). ST237 (100 mg, 0.33 mmol) and aniline (120 μL, 1.31 mmol) in EtOH (6.5 mL) were heated for 13 h at 80° C. The pure product crystallized out of the reaction. After cooling down to room temperature, the product was isolated by suction filtration, washed with cold EtOH to yield a light yellow solid. The filtrate was concentrated and further product crystallized out of the solution to yield ST239 as a light yellow solid (52.0 mg, 0.13 mmol, 40%); ¹H NMR (400 MHz, DMSO-d₆) δ 12.87 (br s, 1H), 10.52 (s, 1H), 9.34 (br s, 1H), 9.23 (br s, 1H), 8.73 (d, 7=1.8 Hz, 1H), 8.10 (dd, 7=1.8, 8.8 Hz, 1H), 7.60-7.66 (m, 2H), 7.37-7.44 (m, 2H), 7.34 (d, 7=8.8 Hz, 1H), 7.16-7.25 (m, 1H); ¹³C/DEPTQ NMR (400 MHz, DMSO-d₆) δ 161.8, 151.7, 143.6, 138.8, 136.9, 133.2, 129.1 (2C), 124.9, 122.1, 119.4 (2C), 111.7, 88.4; ESI-MS m/z 362.9 [M+H]⁺; HRMS-ESI (m/z): [M+H]⁺: calcd. for C₁₄H₁₂IN₄: 363.0101, found: 363.0101; HPLC: System 1: t_{R}⁼15.7 min, purity 99%, System 2: t_{R}⁼12.1 min, purity 99%.

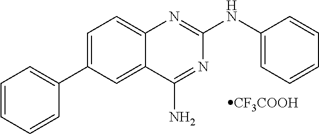

N²,6-Diphenylquinazoline-2,4-diamine×TFA (ST240). Phenylboronic acid (25.6 mg, 0.21 mmol), Na₂CO₃ (89.0 mg, 0.84 mmol) and Pd(dppf)Cl₂ (15.4 mg, 0.021 mmol) were dissolved in a dioxane/H₂O mixture (4:1, 5 mL) in a microwave vial. ST239 (38.0 mg, 0.11 mmol) was added to the mixture and the reaction was heated under argon atmosphere at 80° C. for 3 h. After the reaction has cooled to room temperature, water was added and the aqueous phase was extracted with ethyl acetate three times. The combined organic layers were washed with NaCl, dried over Na₂SO₄ and evaporated. The crude product was purified via preparative HPLC (acetonitrile in 0.1% aqueous trifluoroacetic acid, 5% to 60%) to yield ST240 as a white solid (21.0 mg, 47%); ¹H NMR (400 MHz, DMSO-d₆) δ 13.51 (br s, 1H), 10.79 (s, 1H), 9.32 (br s, 1H), 9.12 (brs, 1H), 8.63 (d, J=1.8 Hz, 1H), 8.20 (dd, J=1.8, 8.7 Hz, 1H), 7.77-7.84 (m, 2H), 7.67-7.74 (m, 2H), 7.59 (d, J=8.7 Hz, 1H), 7.49-7.56 (m, 2H), 7.38-7.46 (m, 3H), 7.15-7.23 (m, 1H); ¹³C/DEPTQ NMR (400 MHz, DMSO-de) δ 163.1, 159.0, 158.7, 152.0, 138.3, 137.5, 136.1, 133.9, 129.1 (2C), 129.0 (2C), 128.0, 126.6 (2C), 124.5, 122.3, 121.8, 118.3, 110.2; ESI-MS m/z 313.0 [M+H]⁺. HRMS-ESI (m/z): [M+H]⁺: calcd. for C₂₀H₁₆7₄: 313.1448, found: 313.1455; HPLC: System 1: t_{R}=17.0 min, purity 99%, System 2: t_{R}=13.2 min, purity 99%.

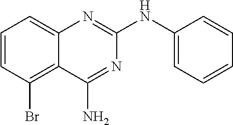 [SMILES: Nc1nc(Nc2ccccc2)nc2cccc(Br)c12]

5-Bromo-N²-phenylquinazoline-2,4-diamine (AS94). The crude material was purified by silica gel chromatography (CH₂Cl₂/MeOH, 30:1 v/v) to obtain AS94 as a light beige solid (30.3 mg, 58%); ¹H NMR (600 MHz, DMSO-d₆) δ 9.13 (s, 1H), 7.95-7.85 (m, 2H), 7.55 (br s, 2H), 7.45-7.38 (m, 3H), 7.29-7.22 (m, 2H), 6.95-6.88 (m, 1H); ¹³C NMR (150 MHz, DMSO-d₆) δ 161.4, 156.9, 155.1, 141.5, 133.6, 128.8 (2C), 128.1, 126.4, 121.3, 119.3 (2C), 118.0, 110.2; ESI-MS m/z 315.4 [M+H]⁺; HRMS-ESI (m/z): [M+H]⁺: calcd. for C₁₄H₁₂BrN₄: 315.0240, found: 315.0251; HPLC: System 1: t_{R}=15.4 min, purity 98%, System 2: t_{R}=14.4 min, purity 98%.

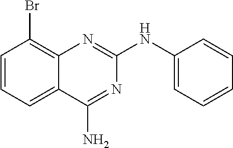 [SMILES: Nc1nc(Nc2ccccc2)nc2c(Br)cccc12]

8-Bromo-N²-phenylquinazoline-2,4-diamine (AS241). The crude material was purified by silica gel chromatography (CH₂Cl₂/MeOH, 30:1 v/v) to give AS241 as a light beige solid (41.1 mg, 84%); ¹H NMR (600 MHz, DMSO-d₆) δ 9.13 (s, 1H), 8.14 (d, J=8.0 Hz, 2H), 8.11 (dd, J=8.1, 0.9 Hz, 1H), 7.96 (dd, J=IN 1.0 Hz, 1H), 7.59 (br s, 2H), 7.27 (t, J=7.9 Hz, 2H), 7.09 (t, J=7.8 Hz, 1H), 6.92 (t, J=7.3 Hz, 1H); ¹³C NMR (90 MHz, DMSO-d₆) δ 162.3, 157.3, 149.0, 141.2, 135.9, 128.3 (2C), 123.3, 121.9, 120.7, 120.1, 118.7 (2C), 112.8; ESI-MS m/z 314.7 [M+H]⁺; HRMS-ESI (m/z): [M+H]⁺: calcd. for C₁₄H₁₂BrN₄: 315.0240, found: 315.0244; HPLC: System 1: t_{R}=16.0 min, purity 99%, System 2: t_{R}=14.0 min, purity 99%.

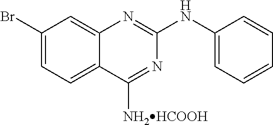

7-Bromo-N²-phenylquinazoline-2,4-diamine×HCOOH (AS436). The crude material was purified by preparative HPLC (acetonitrile in 0.1% aqueous HCOOH, 5% to 95%) to give AS436 as a light beige solid (32.5 mg, 76%); ³H NMR (600 MHz, DMSO-d₆) δ 9.12 (s, 1H), 8.14 (s, 1H), 8.03 (d, J=8.6 Hz, 1H), 7.90 (d, J=7.9 Hz, 2H), 7.63 (br s, J=36.4 Hz, 2H), 7.57 (br s, 1H), 7.30 (d, J=8.6 Hz, 1H), 7.25 (t, J=7.8 Hz, 2H), 6.91 (t, J=7.3 Hz, 1H); ¹³C NMR (100 MHz, DMSO-d₆) δ 163.5, 162.5, 158.2, 153.2, 141.5, 128.7 (2C), 127.3, 126.9, 126.2, 124.5, 121.3, 119.4 (2C); ESI-MS m/z 315.0 [M+H]⁺; HRMS-ESI (m/z): [M−H]⁻: calcd. for C₁₄H₁₂BrN₄: 313.0094, found: 313.0098; HPLC: System 1: t_{R}=16.3 min, purity 99%, System 2: t_{R}=16.6 min, purity 99%

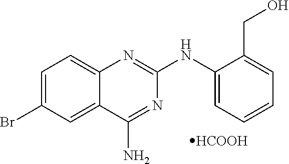 [SMILES: Nc1nc(Nc2ccccc2CO)nc2ccc(Br)cc12 ; "·HCOOH"]

6-Bromo-N²-(2-hydroxymethyl)phenylquinazoline-2,4-diamine×HCOOH (DD283) The crude material was purified by preparative HPLC (acetonitrile in 0.1% aqueous HCOOH, 15% to 37%) to give DD283 as a white solid (6.5 mg, 24%); ³H NMR (600 MHz, DMSO-dd) δ 8.59 (br s, 1H), 8.34 (d, J=2.2 Hz, 1H), 8.32 (br d, J=7.9 Hz, 1H), 7.73 (br s, 2H), 7.69 (dd, J=8.9, 2.2 Hz, 1H), 7.31 (d, J=8.8 Hz, 1H), 7.29-7.24 (m, 2H), 6.97 (br t, J=7.4 Hz, 1H), 5.57 (br s, 1H), 4.57 (s, 2H). ¹³C NMR (151 MHz, DMSO-76) δ 162.92, 161.38, 157.18, 138.97, 135.56, 127.90, 127.42, 127.26, 125.88, 121.46, 120.85, 112.97, 112.62, 99.41, 62.09. ESI-MS m/z 345.0 [M+H]⁺; HRMS-ESI (m/z): [M+H]⁺: calcd. for C₁₅H₁₄BrN₄O: 345.0345, found 345.0349; HPLC: System 1: t_{R}=14.7 min, purity 96%, System 2: t_{R}=12.5 min, purity 96%.

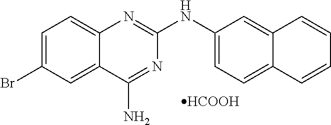 [SMILES: Nc1nc(Nc2ccc3ccccc3c2)nc2ccc(Br)cc12 ; "·HCOOH"]

6-bromo-N2-(naphthalen-2-yl)quinazoline-2,4-diamine×HCOOH (DD290). The crude material was purified by preparative HPLC (acetonitrile in 0.1% aqueous HCOOH, 5% to 57%) to give DD290 as a beige white solid (8 mg, 28%); ³H NMR (600 MHz, DMSO-76) δ 9.32 (s, 1H), 8.74 (s, 1H), 8.38 (d, J=2.2 Hz, 1H), 8.22 (br s, 1H), 7.85-7.75 (m, 4H), 7.73 (dd, J=8.9, 2.2 Hz, 1H), 7.65 (br s, 2H), 7.48-7.40 (m, 2H), 7.31 (t, 7=7.5 Hz, 1H). ¹³C NMR (151 MHz, DMSO-76) δ 161.71, 157.95, 150.98, 139.33, 136.04, 134.35, 129.01, 128.06, 128.03, 127.70, 127.40, 126.43, 126.37, 123.73, 121.13, 113.91, 113.45, 113.08. ESI-MS m/z 365.0 [M+H]⁺; HRMS-ESI (m/z): [M+H]⁺: calcd. for C₁₈H₁₄BrN₄: 365.0396, found 365.0396; HPLC: System 1: t_{R}=17.2 min, purity 95%, System 2: t_{R}=14.6 min, purity 95%.

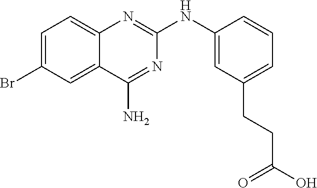 [SMILES: Nc1nc(Nc2cccc(CCC(=O)O)c2)nc2ccc(Br)cc12]

3-(3-((4-amino-6-bromoquinazolin-2-yl)amino)phenyl)propanoic acid (DD291). The crude material was purified by preparative HPLC (acetonitrile in 0.1% aqueous HCOOH, 15% to 47%) to give DD291 as a beige white solid (35 mg, 39%); ³H NMR (600 MHz, DMSO-76) δ 8.97 (s, 1H), 8.34 (d, J=2.3 Hz, 1H), 8.26 (br s, 1H), 7.77 (s, 1H), 7.73 (br d, J=8.1 Hz, 1H), 7.68 (dd, J=8.9, 2.2 Hz, 1H), 7.56 (br s, 2H), 7.33 (d, J=8.9 Hz, 1H), 7.14 (t, 7=7.8 Hz, 1H), 6.76 (d, 7=7.5 Hz, 1H), 2.80 (t, 7=7.6 Hz, 2H), 2.60-2.52 (m, 2H). ¹³C NMR (151 MHz, DMSO-76) δ 183.66, 161.10, 157.37, 150.47, 141.09, 140.76, 135.43, 128.06, 127.41, 125.84, 120.53, 118.52, 116.51, 112.66, 112.46, 39.37, 30.58. ESI-MS m/z 387.0 [M+H]⁺; HRMS-ESI (m/z): [M+H]⁺: calcd. for C₁₇H₁₆BrN₄O₂: 387.0451, found 387.0454; HPLC: System 2^{ST}: t_{R}=12.1 min, purity 98%.

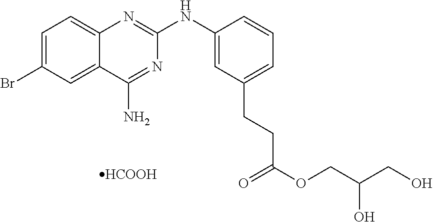 [SMILES: Nc1nc(Nc2cccc(CCC(=O)OCC(O)CO)c2)nc2ccc(Br)cc12 ; "·HCOOH"]

2,3-dihydroxypropyl-(3-((4-amino-6-bromoquinazolin-2-yl)amino)phenyl)propanoate HCOOH (DD294). DD291 (14.0 mg, 1 eq, 36 μmol) was suspended in dry dichloromethane (1 mL). Catalytic amount of DMF was added, followed by glycerol (132 μL, 50 eq, 1.81 mmol). The mixture was stirred vigorously in an ice-water-bath when thionyl chloride (13 μL, 5 eq, 181 μmol) was added dropwise. The reaction mixture was stirred for 6 hours at room temperature. The solvent was rotary evaporated, resulting residue diluted with toluene and the mixture was concentrated under reduced pressure. This procedure was repeated twice. Then it was diluted with 0. IN HCl solution, the aqueous phase was washed with dichloromethane (3×), adjusted to a pH of 9 with sat. NaHCO₃/Na₂CO₃ solution and extracted three times with dichloromethane. The crude material was purified by preparative HPLC (acetonitrile in 0.1% aqueous HCOOH, 15% to 37%) to give DD294 as a white solid (4.6 mg, 27%) after lyophilisation; ¹H NMR (600 MHz, DMSO-76) δ 8.97 (s, 1H), 8.34 (d, J=2.2 Hz, 1H), 8.30 (s, 1H), 7.78-7.75 (m, 1H), 7.75-7.73 (m, 1H), 7.69 (dd, J=8.8, 2.2 Hz, 1H), 7.56 (s, 2H), 7.34 (d, J=8.8 Hz, 1H), 7.15 (t, J=7.8 Hz, 1H), 6.77 (dt, 7=7.6, 1.3 Hz, 1H), 4.89 (s, 1H), 4.64 (s, 1H), 4.06 (dd, 7=11.1, 4.2 Hz, 1H), 3.92 (dd, J=11.1, 6.6 Hz, 1H), 3.64 (qd, 7=6.1, 4.3 Hz, 1H), 3.35 (dd, J=11.0, 5.4 Hz, 1H), 3.32 (dd, 7=11.0, 6.1 Hz, 1H), 2.84 (t, 7=7.7 Hz, 2H), 2.64 (t, 7=7.7 Hz, 2H). ¹³C NMR (151 MHz, DMSO-76) δ 172.29, 161.19, 157.45, 150.55, 141.21, 140.50, 135.52, 128.22, 127.49, 125.93, 120.58, 118.58, 116.69, 112.76, 112.55, 69.23, 65.70, 62.59, 35.09, 30.50. ESI-MS m/z 461.1 [M+H]⁺; HRMS-ESI (m/z): [M+H]⁺: calcd. for C₂₀H₂₂BrN₄O₄: 461.0819, found 461.0818; HPLC: System 1: t_{R}=15.0 min, purity 95%, System 2: t_{R}=11.6 min, purity 95%.

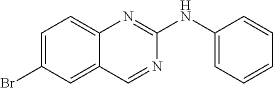 [SMILES: Brc1ccc2nc(Nc3ccccc3)ncc2c1]

6-Bromo-/V-phenylquinazolin-2-amine (DD288). 90.0 μL aniline (4 eq, 0.99 mmol) were added to a solution of 60.0 mg of 6-bromo-2-chloroquinazoline (1 eq, 0.25 mmol) in anhydrous ethanol (4 mL) in a pressure tube and the reaction mixture was stirred at 80° C. for 6 h. The solvent was rotary evaporated and the crude material was purified by silica gel flash chromatography (EtOAc/hexane, 3:1 v/v) giving DD288 as a yellow solid (72.4 mg, 98%); ¹H NMR (600 MHz, CDCl₃) δ 9.02-8.97 (m, 1H), 7.86 (d, J=2.2 Hz, 1H), 7.82-7.77 (m, 3H), 7.62 (d, J=8.9 Hz, 1H), 7.46 (br s, 1H), 7.40-7.36 (m, 2H), 7.09 (tt, J=7.5, 1.0 Hz, 1H); ¹³C NMR (150 MHz, CDCl₃) δ 160.8, 156.9, 150.3, 139.2, 137.6, 129.4, 129.0, 128.2 (2C), 122.9, 121.8, 119.2 (2C), 116.5; ESI-MS m/z 299.9 [M+H]⁺; HRMS-ESI (m/z): [M+H]⁺: calcd. for C₁₄H₁₁BrN₃: 300.0131, found: 300.0133; HPLC: System 1: t_{R}=21.5 min, purity 99%, System 2: t_{R}=20.3 min, purity 99%.

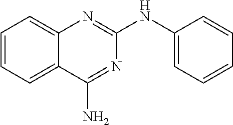 [SMILES: Nc1nc(Nc2ccccc2)nc2ccccc12]

N²-Phenylquinoline-2,4-diamine (AS224). A solution of 2-chloroquinazoline-4-amine and aniline was stirred in a pressure tube at 80° C. for 16 h. The solvent was rotary evaporated and the crude material was treated with saturated, aq. NaHCO₃, extracted with CH₂Cl₂ (3×), dried (MgSO₄) and the solvent was rotary evaporated. The crude was purified by silica gel chromatography (CH₂Cl₂/MeOH, 30:1 v/v) to give AS224 as a colorless solid (8.30 mg, 43%); ¹H NMR (600 MHz, DMSO-d₆) δ 9.96 (br s, 1H), 8.22 (d, J=8.2 Hz, 1H), 7.94 (br s, 2H), 7.72-7.67 (m, 1H), 7.66-7.62 (m, 1H), 7.47-7.42 (m, 4H), 7.40-7.35 (m, 1H), 7.25-7.19 (m, 1H), 6.20 (s, 1H); ¹³C NMR (150 MHz, DMSO-d₆) δ 156.4, 152.6, 139.4, 138.3, 132.7, 130.0, 125.4, 123.8, 123.7, 123.3, 119.8, 114.9, 86.6; ESI-MS m/z 235.9 [M+H]⁺; HRMS-ESI (m/z): [M+H]⁺: calcd. for C₁₅H₁₄N₃: 236.1182, found: 236.1180; HPLC: System 1: t_{R}⁼15.5 min, purity 99%, System 2: t_{R}⁼14.3 min, purity 99%.

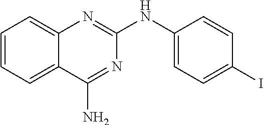 [SMILES: Nc1nc(Nc2ccc(I)cc2)nc2ccccc12]

N²-(4-Iodophenyl)quinazoline-2,4-diamine (AS077). The crude material was purified by silica gel chromatography (CH₂Cl₂/MeOH, 30:1 v/v) to obtain AS077 as a colorless solid (21.0 mg, 35%); ¹H NMR (600 MHz, CDCl₃) δ 7.67-7.63 (m, 1H), 7.63-7.57 (m, 4H), 7.57-7.51 (m, 2H), 7.25-7.20 (m, 1H), 7.09 (br s, 1H), 5.51 (br s, 2H); ¹³C NMR (150 MHz, CDCl₃) 161.9, 156.3, 151.8, 134.0, 137.6, 133.6, 126.6, 122.8, 121.7, 121.1, 111.0, 84.3; ESI-MS m/z 363.4 [M+H]⁺; HRMS-ESI (m/z): [M+H]⁺: calcd. for C₁₄H₁₂IN₄: 363.0101, found: 363.0112; HPLC: System 1: t_{R}⁼16.5 min, purity 98%, System 2: t_{R}⁼15.5 min, purity 98%.

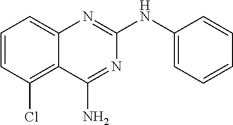 [SMILES: Nc1nc(Nc2ccccc2)nc2cccc(Cl)c12]

5-Chloro-N²-phenylquinazoline-2,4-diamine×HCOOH (AS098/AS240). The crude material was purified by preparative HPLC (acetonitrile in 0.1% aq. HCOOH, 5% to 95%) to give AS098 as a light beige solid (27.4 mg, 71%); ³H NMR (360 MHz, DMSO-d₆) δ 9.07 (br s, 1H), 7.94-7.79 (m, 2H), 7.67-7.40 (m, 3H), 7.34 (dd, J=8.4, 1.1 Hz, 1H), 7.28-7.14 (m, 3H), 6.95-6.84 (m, 1H); ¹³C NMR (90 MHz, DMSO-d₆) δ 160.9, 156.8, 154.7, 141.0, 132.6, 129.1, 128.3, 125.3, 123.6, 120.9, 118.9, 108.6; ESI-MS m/z 271.4 [M+H]⁺; HRMS-ESI (m/z): [M+H]⁺: calcd. for C₁₄H₁₂ClN₄: 271.0745, found: 271.0746; HPLC: System 1: t_{R}⁼15.1 min, purity 98%, System 2: t_{R}⁼14.2 min, purity 99%.

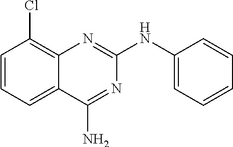 [SMILES: Nc1nc(Nc2ccccc2)nc2c(Cl)cccc12]

8-Chloro-N²-phenylquinazoline-2,4-diamine×HCOOH (AS328). The crude material was purified by preparative HPLC (acetonitrile in 0.1% aq. HCOOH, 5% to 95%) to give AS328 as a colorless solid (35.0 mg, 55%); ³H NMR (600 MHz, CDCl₃) δ 9.12 (s, 1H), 8.18-8.07 (br s, 1H), 8.11 (m, 2H), 8.06 (dd, J=8.1, 1.2 Hz, 1H), 7.77 (dd, 7.6, 1.2 Hz, 1H), 7.60 (br s, 2H), 7.31-7.22 (m, 2H), 7.13 (t, J=7.8 Hz, 1H), 6.95-6.84 (m, 1H); ¹³C NMR (90 MHz, DMSO) δ 162.2, 157.3, 148.1, 141.2, 132.5, 128.6, 128.2, 122.7, 121.1, 120.7, 118.6, 112.7; ESI-MS m/z 271.8 [M+H]⁺; HRMS-ESI (m/z): [M−H]⁺: calcd. for C₁₄H₁₂ClN₄: 269.0599, found: 269.0600; HPLC: System 1: t_{R}⁼14.7 min, purity >99%, System 2: t_{R}⁼13.6 min, purity 99%.

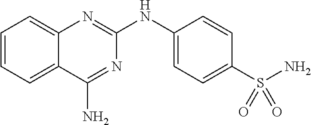 [SMILES: Nc1nc(Nc2ccc(S(N)(=O)=O)cc2)nc2ccccc12]

4-((4-Aminoquinazolin-2-yl)amino)benzenesulfonamide (AS197). The crude material was purified by silica gel chromatography (CH₂Cl₂/MeOH/25% aq. NH₄OH, 10:1:0.1 v/v/v) to give AS197 as a light yellow solid (25.6 mg, 48%); ¹H NMR (600 MHz, DMSO-d₆) δ 9.43 (br s, 1H), 8.15-8.06 (m, 3H), 7.71-7.68 (m, 2H), 7.66 (br s, 2H), 7.64 (ddd, J=8.3, 7.0, 1.3 Hz, 1H), 7.47 (d, J=7.8 Hz, 1H), 7.26-7.19 (m, 1H), 7.14 (br s, 2H); ¹³C NMR (90 MHz, DMSO-di) δ 162.3, 156.7, 150.9, 144.6, 135.2, 133.0, 126.3, 125.2, 123.7, 122.1, 117.7, 111.4; ESI-MS m/z 316.3 [M+H]⁺; HRMS-ESI (m/z): [M+Na]⁺: calcd. for C₁₄H₁₄N₅O₂S: 338.0682, found: 338.0689; HPLC: System 1: t_{R}=12.8 min, purity 97%, System 2: t_{R}=11.7 min, purity 97%.

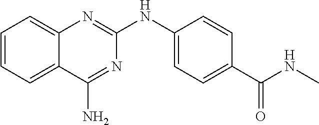 [SMILES: CNC(=O)c1ccc(Nc2nc(N)c3ccccc3n2)cc1]

4-((4-Aminoquinazolin-2-yl)amino)-N-methylbenzamide (AS198). The crude material was purified by silica gel chromatography (CH₂Cl₂/MeOH/25% aq. NH₄OH, 10:1:0.1 v/v/v) to give AS198 as a colorless solid (48.9 mg, 98%); ¹H NMR (600 MHz, DMSO-d₆) δ 9.30 (br s, 1H), 8.24-8.18 (q, J=4.2 Hz, 1H), 8.11 (dd, J=8.1, 0.8 Hz, 1H), 8.02-7.97 (m, 2H), 7.77-7.74 (m, 2H), 7.74-7.47 (m, 2H), 7.65-7.60 (m, 1H), 7.45 (d, J=8.3 Hz, 1H), 7.24-7.19 (m, 1H), 2.78 (d, J=4.5 Hz, 3H); ¹³C NMR (90 MHz, DMSO-d₆) δ 166.4, 162.3, 156.7, 150.7, 143.9, 133.0, 127.6, 126.3, 125.0, 123.7, 121.9, 117.6, 111.3, 26.2; ESI-MS m/z 294.3 [M+H]⁺; HRMS-ESI (m/z): [M+Na]⁺: calcd. for C₁₆H₁₆N₅O: 316.1174, found: 316.1173; HPLC: System 1: t_{R}=13.7 min, purity 99%, System 2: t_{R}=11.8 min, purity 99%.

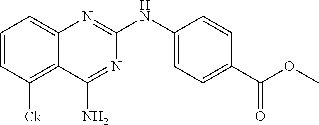

Methyl 4-((4-amino-5-chloroquinazolin-2-yl)amino)benzoate (AS228). The crude material was purified by silica gel chromatography (CH₂Cl₂/MeOH, 20:1 v/v) to give AS228 as a light beige solid (5.80 mg, 25%); ¹H NMR (360 MHz, DMSO-d₆) δ 9.60 (br s, 1H), 8.07-8.00 (m, 2H), 7.88-7.81 (m, 2H), 7.71 (br s, 2H), 7.55 (dd, J=8.3, 7.8 Hz, 1H), 7.41 (dd, J=8.4, 1.1 Hz, 1H), 7.26 (dd, J=7.6, 1.1 Hz, 1H), 3.80 (s, 3H); ¹³C NMR (150 MHz, DMSO-d₆) δ 166.5, 161.5, 156.6, 154.6, 146.1, 133.4, 130.4, 129.7, 125.7, 124.9, 121.8, 118.4, 109.2, 52.1; ESI-MS m/z 328.9 [M+H]⁺; HRMS-ESI (m/z): [M+H]⁺: calcd. for C₁₆H₁₄ClN₄O₂: 329.0800, found: 329.0799; HPLC: System 1: t_{R}=16.7 min, purity 95%, System 2: t_{R}=15.0 min, purity 95%.

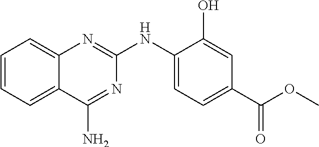 [SMILES: COC(=O)c1ccc(Nc2nc(N)c3ccccc3n2)c(O)c1]

Methyl 4-((4-aminoquinazolin-2-yl)amino)-3-hydroxybenzoate (JT11). The crude material was purified by silica gel chromatography (CH₂Cl₂/MeOH, 30:1 v/v) to give JT11 as a colorless solid (11.6 mg, 9%); ¹H NMR (600 MHz, DMSO-d₆) δ 11.39 (br s, 1H), 8.38 (d, J=8.4 Hz, 1H), 8.16-8.09 (m, 2H), 7.79 (br s, 2H), 7.66 (ddd, J=8.3, 7.0, 1.4 Hz, 1H), 7.49-7.39 (m, 3H), 7.26-7.23 (m, 1H), 3.81 (s, 3H); ¹³C NMR (90 MHz, DMSO-d₆) δ 166.1, 162.6, 156.5, 150.2, 145.7, 133.9, 133.3, 124.9, 123.8, 122.4, 122.4, 121.1, 118.4, 115.9, 111.3, 51.7; ESI-MS m/z 311.1 [M+H]⁺; HRMS-ESI (m/z): [M+H]⁺: calcd. for C₁₆H₁₅N₄O₃: 311.1139, found: 311.1141; HPLC: System 1: t_{R}=15.3 min, purity 95%, System 2: t_{R}=12.9 min, purity 95%.

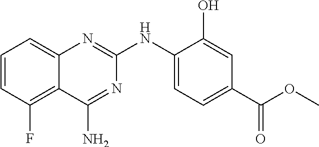 [SMILES: COC(=O)c1ccc(Nc2nc(N)c3c(F)cccc3n2)c(O)c1]

Methyl 4-((4-amino-5-fluoroquinazolin-2-yl)amino)-3-hydroxybenzoate (AS202). The crude material was purified by silica gel chromatography (CH₂Cl₂/MeOH/aq. NH₄OH 25%, 10:1:0.1 v/v/v) to give AS202 as a yellow solid (8.02 mg, 17%); ³H NMR (600 MHz, DMSO-d₆) δ 10.87 (br s, 1H), 8.53 (d, J=8.4 Hz, 1H), 7.99 (s, 1H), 7.93 (br s, 2H), 7.66-7.59 (m, 1H), 7.49-7.43 (m, 2H), 7.32-7.19 (m, 2H), 7.06-6.97 (m, 1H), 3.81 (s, 3H); ¹³C NMR (90 MHz, DMSO-d₆) δ 166.5, 160.4 (d, J_{CF}=3.8 Hz), 159.5 (d, J_{CF}=252.4 Hz), 157.2, 153.6, 145.8, 143.0 (d, J_{CF}=40.2 Hz), 133.9 (d, J_{CF}=11.3 Hz), 133.8, 122.7, 121.8 (d, J_{CF}=3.2 Hz), 118.5, 115.6, 108.1 (d, J_{CF}=22.5 Hz), 101.5 (d J_{CF}=11.3 Hz), 52.1; ESI-MS m/z 329.1 [M+H]⁺; HRMS-ESI (m/z): [M+H]⁺: calcd. for C₁₆H₁₅N₄O₃: 329.1044, found: 329.1041; HPLC: System 1: t_{R}⁼15.4 min, purity 95%, System 2: t_{R}⁼13.3 min, purity 95%.

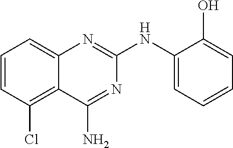 [SMILES: Nc1nc(Nc2ccccc2O)nc2cccc(Cl)c12]

2-((4-Amino-5-chloroquinazolin-2-yl)amino)phenol (JT12). The crude material was purified by silica gel chromatography (CH₂Cl₂/MeOH, 30:1 v/v) to give JT12 as a colorless solid (8.30 mg, 43%); ³H NMR (400 MHz, DMSO-d₆) δ 10.68 (br s, 1H), 8.07 (br s, 1H), 8.05-7.97 (m, 1H), 7.84 (br s, 2H), 7.53 (dd, J=8.4, 7.7 Hz, 1H), 7.34 (dd, J=8.4, 1.2 Hz, 1H), 7.24 (dd, J=7.6, 1.2 Hz, 1H), 6.92-6.83 (m, 2H), 6.83-6.73 (m, 1H); ¹³C NMR (150 MHz, DMSO-d₆) δ 161.1, 156.5, 153.7, 146.9, 133.1, 129.3, 128.5, 124.7, 124.2, 122.7, 120.5, 119.2, 116.2, 108.5; ESI-MS m/z 287.0 [M+H]⁺; HRMS-ESI (m/z): [M+H]⁺: calcd. for C₁₄H₁₂ClN₄O₃: 287.0694, found: 287.0696; HPLC: System 1: t_{R}⁼14.6 min, purity 96%, System 2: t_{R}⁼12.8 min, purity 95%.

### 6-Isopropyl-N²-phenylquinazoline-2,4-diamine (ST238)

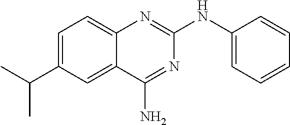 [SMILES: CC(C)c1ccc2nc(Nc3ccccc3)nc(N)c2c1]

ST236 (50 mg, 0.225 mmol) and aniline (82 μL, 0.90 mmol) in EtOH (4.5 mL) were heated at 80° C. for 13 h. Extraction with DCM/NaHCO₃, washed with NaCl, dried over Na₂SO₄ and purification with flash chromatography (DCM/MeOH 95/5+NH₃) gave ST238 (62 mg, 98% yield).

¹H NMR (400 MHz, DMSO-d₆) δ 1.26 (d, J=7.0 Hz, 6H), 2.94 (sept, J=7.0 Hz, 1H), 6.82-6.88 (m, 1H), 7.20-7.26 (m, 2H), 7.34 (d, J=8.6 Hz, 1H), 7.38 (br s, 2H), 7.50 (dd, J=2, 8.7 Hz, 1H), 7.90-7.95 (m, 3H), 8.83 (s, 1H); ¹³C/DEPTQ NMR (400 MHz, DMSO-de) δ 23.82, 24.01, 33.36, 110.97, 118.35 (×2), 120.13 (×2), 125.16, 128.21 (×2), 131.98, 141.64, 141.72, 149.99, 156.82, 161.98; ESI-MS m/z 279.0 [M+H]⁺; HRMS-ESI (m/z): [M+H]⁺: calcd. for C₁₇H₁₉N₄: 279.1604, found: 279.1609; HPLC: System 1: t_{R}=16.8 min, purity 99%, System 2: t_{R}=12.9 min, purity 99%.

¹³C/DEPTQ NMR (400 MHz, DMSO-d₆) δ 110.18, 118.30, 121.84, 122.33, 124.45, 126.61 (×2), 127.96, 128.98 (×2), 129.09 (×2), 133.87, 136.12, 137.53, 138.27, 151.99, 158.68, 158.99, 163.14; ESI-MS m/z 313.0 [M+H]⁺. HRMS-ESI (m/z): [M+H]⁺: calcd. for C₂₀H₁₇N₄: 313.1448, found: 313.1455; HPLC: System 1: t_{R}=17.0 min, purity 99%, System 2: t_{R}=13.2 min, purity 99%.

RP-analytical HPLC-ST were performed on a AGILENT 1200 series HPLC system employing a DAD detector and detection at 200, 220, 230 or 254 nm. HPLC column was a ZORBAX ECLIPSE XDB-C8 (4.6×150 mm, 5 μm) with a flow rate of 0.5 m1/min. As solvent systems, methanol/H₂O or CH₃CN/H₂O binary grade systems were applied:
System 1^{ST}: eluent, methanol/0.1% aq. formic acid; 10% methanol for 3 min, to 100% in 15 min, 100% for 6 min, to 10% in 3 min, 10% methanol for 3 min.
System 2^{ST}: eluent, CH₃CN/0.1% aq. trifluoroacetic acid; 5-80% CH₃CN in 18 min, then 80-95% in 2 min, 95% for 2 min, to 5% in 3 min, 5% CH₃CN for 3 min.

Further examples may be:

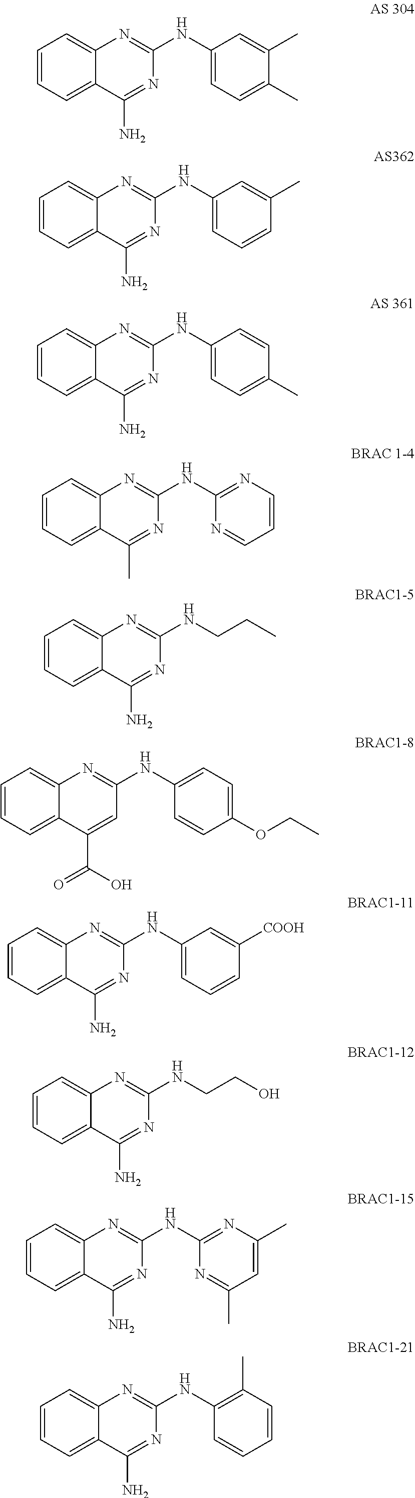

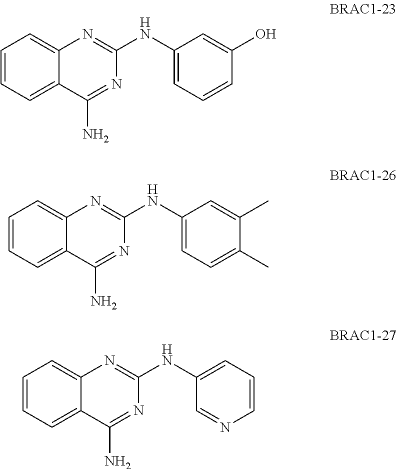 [SMILES: Nc1nc(Nc2cccc(O)c2)nc2ccccc12 ; Cc1ccc(Nc2nc(N)c3ccccc3n2)cc1C ; Nc1nc(Nc2cccnc2)nc2ccccc12]

TABLE 3
Biological data for the example compounds expressed as
attenuation of the maximum efficacy of norepinephrine.^{a}

| compound | attenuation of Eₘₐₓ^{b} |
|---|---|
| AS408 | A |
| AS241 | B |
| AS436 | B |
| AS094 | B |
| AS224 | C |
| AS328 | B |
| AS098 | B |
| DD282 | A |
| DD284 | C |
| DD293 | A |
| DD288 | B |
| DD283 | C |
| DD290 | C^{c} |
| DD291 | C |
| DD294 | C |
| ST238 | B |
| ST239 | A |
| ST240 | B |
| AS077 | nd |
| AS198 | C |
| AS197 | C |
| AS228 | C |
| JT11 | C^{c} |
| JT12 | C |
| AS202 | C |

^{a}Maximum efficacy was determined using an enzyme fragment complementation based assay to measure the amount of arrestin recruitment stimulated by norepinephrine after preincubation with 30 μM of the test compound.
^{b}Attenuation of Eₘₐₓ differentiated in the classes A: 71-100%; B: 31-70%; C: <30%.
^{c}Attenuating effect at 10 μM.
nd: not determined

### REFERENCES

1. Kruse, A. C. et al. Activation and allosteric modulation of a muscarinic acetylcholine receptor. Nature 504, 101-106, doi:10.1038/nature12735 (2013).
2. Digby, G. J., Shirey, J. K. & Conn, P. J. Allosteric activators of muscarinic receptors as novel approaches for treatment of CNS disorders. Mol Biosyst 6, 1345-1354, doi:10.1039/c002938f (2010).
3. Mohr, K., Trankle, C. & Holzgrabe, U. Structure/activity relationships of M2 muscarinic allosteric modulators. Receptors Channels 9, 229-240 (2003).
4. Liu, X. et al. Mechanism of intracellular allosteric beta2AR antagonist revealed by X-ray crystal structure. Nature 548, 480-484, doi:10.1038/nature23652 (2017).
5. Ahn, S. et al. Allosteric “beta-blocker” isolated from a DNA-encoded small molecule library. Proceedings of the National Academy of Sciences of the United States of America 114, 1708-1713, doi: 10.1073/pnas. 1620645114 (2017).
6. Lu, J. et al. Structural basis for the cooperative allosteric activation of the free fatty acid receptor GPR40. Nat Struct Mol Biol 24, 570-577, doi: 10.1038/nsmb.3417 (2017).
7. Roth, C. B., Hanson, M. A. & Stevens, R. C. Stabilization of the human beta2-adrenergic receptor TM4-TM3-TM5 helix interface by mutagenesis of Glu122(3.41), a critical residue in GPCR structure. J Mol Biol 376, 1305-1319, doi: 10.1016/j.jmb.2007.12.028 (2008).
8. Pettersen, E. F. et al. UCSF Chimera-A visualization system for exploratory research and analysis. J Comput Chem 25, 1605-1612, doi: 10.1002/jcc.20084 (2004).
9. Lomize, M. A., Lomize, A. L., Pogozheva, F D. & Mosberg, H. F OPM: orientations of proteins in membranes database. Bioinformatics 22, 623-625, doi:10.1093/bioinformatics/btk023 (2006).
10. Wolf, M. G., Floefling, M., Aponte-Santamaria, C., Grubmuller, H. & Groenhof, G. g_membed: Efficient insertion of a membrane protein into an equilibrated lipid bilayer with minimal perturbation. J Comput Chem 31, 2169-2174, doi: 10.1002/jcc.21507 (2010).
11. Case, D. A. et al. AMBER17. University of California, San Francisco (2017).
12. Wang, J., Wolf, R. M., Caldwell, J. W., Kollman, P. A. & Case, D. A. Development and Testing of a General Amber Force Field. J Comput Chem 25, 1157-1174 (2004).
13. Dickson, C. J. et al. Lipid14: The Amber Lipid Force Field. J Chem Theory Comput 10, 865-879, doi:10.1021/ct4010307 (2014).
14. Maier, J. A. et al. ff14SB: Improving the Accuracy of Protein Side Chain and Backbone Parameters from ff99SB. J Chem Theory Comput 11, 3696-3713, doi:10.1021/acs.jctc.5b00255 (2015).
15. Berendsen, H. J. C., Grigera, J. R. & Straatsma, T. P. The Missing Term in Effective Pair Potentials. J Phys Chem 91, 6269-6271, doi:10.1021/j100142a004 (1987).
16. Frisch, M. J. et al. Gaussian 09, revision B.01. Gaussian, Inc.: Wallingford Conn., (2009).
17. Bayly, C. I., Cieplak, P., Cornell, W. D. & Kollman, P. A. A Well-Behaved Electrostatic Potential Based Method Using Charge Restraints for Deriving Atomic Charges—the Resp Model. J Phys Chem 97, 10269-10280, doi:DOI 10.1021/j100142a004 (1993).
18. Baaden, M., Burgard, M. & Wipff, G. TBP at the Water-Oil Interface: The Effect of TBP Concentration and Water Acidity Investigated by Molecular Dynamics Simulations. J Phys Chem B 105, 11131-11141, doi: 10.1021/jp011890n (2001).
19. Van Der Spoel, D. et al. GROMACS: fast, flexible, and free. J Comput Chem 26, 1701-1718, doi: 10.1002/jcc.20291 (2005).
20. Abraham, M. J. et al. GROMACS: High performance molecular simulations through multi-level parallelism from laptops to supercomputers. SoftwareX 1-2, 19-25, doi:10.1016/j.softx.2015.06.001 (2015).
21. Rosenbaum, D. M. et al. GPCR engineering yields high-resolution structural insights into beta2-adrenergic receptor function. Science 318, 1266-1273, doi:10.1126/science. 1150609 (2007).
22. Kobilka, B. K. Amino and carboxyl terminal modifications to facilitate the production and purification of a G protein-coupled receptor. Analytical biochemistry 231, 269-271, doi:10.1006/abio.1995.1533 (1995).
23. Caffrey, M. & Cherezov, V. Crystallizing membrane proteins using lipidic mesophases. Nature protocols 4, 706-731, doi:10.1038/nprot.2009.31 (2009).
24. Kabsch, W. Xds. Acta crystallographica. Section D, Biological crystallography 66, 125-132, doi: 10.1107/S0907444909047337 (2010).
25. Emsley, P., Lohkamp, B., Scott, W. G. & Cowtan, K. Features and development of Coot. Acta crystallographica. Section D, Biological crystallography 66, 486-501, doi: 10.1107/S0907444910007493 (2010).
26. Adams, P. D. et al. PHENIX: a comprehensive Python-based system for macromolecular structure solution. Acta crystallographica. Section D, Biological crystallography 66, 213-221, doi: 10.1107/S0907444909052925 (2010).
27. Chen, V. B. et al. MolProbity: all-atom structure validation for macromolecular crystallography. Acta crystallographica. Section D, Biological crystallography 66, 12-21, doi: 10.1107/S0907444909042073 (2010).
28. Whorton, M. R. et al. A monomeric G protein-coupled receptor isolated in a high-density lipoprotein particle efficiently activates its G protein. Proceedings of the National Academy of Sciences of the United States of America 104, 7682-7687, doi:10.1073/pnas.0611448104 (2007).
S. Ahn et al., Small-Molecule Positive Allosteric Modulators of the beta2-Adrenoceptor Isolated from DNA-Encoded Libraries. Mol Pharmacol 94, 850-861 (2018).
S. G. Rasmussen et al., Structure of a nanobody-stabilized active state of the beta(2) adrenoceptor. Nature 469, 175-180 (2011).
K. Leach, P. M. Sexton, A. Christopoulos, Allosteric GPCR modulators: taking advantage of permissive receptor pharmacology. Trends Pharmacol Sci 28, 382-389 (2007).
L. Aurelio et al., Allosteric modulators of the adenosine A1 receptor: synthesis and pharmacological evaluation of 4-substituted 2-amino-3-benzoylthiophenes. J Med Chem 52, 4543-4547 (2009).
A. Srivastava et al., High-resolution structure of the human GPR40 receptor bound to allosteric agonist TAK-875. Nature 513, 124-127 (2014).
J. J. Klicic, R. A. Friesner, S.-Y. Liu, W. C. Guida, Accurate Prediction of Acidity Constants in Aqueous Solution via Density Functional Theory and Self-Consistent Reaction Field Methods. The Journal of Physical Chemistry A 106, 1327-1335 (2002).
A. D. Bochevarov, M. A. Watson, J. R. Greenwood, D. M. Philipp, Multiconformation, Density Functional Theory-Based pKa Prediction in Application to Large, Flexible Organic Molecules with Diverse Functional Groups. J Chem Theory Comput 12, 6001-6019 (2016).
H. S. Yu, M. A. Watson, A. D. Bochevarov, Weighted Averaging Scheme and Local Atomic Descriptor for pKa Prediction Based on Density Functional Theory. J Chem Inf Model 58, 271-286 (2018).
T. Darden, D. York, L. Pedersen, Particle Mesh Ewald—an N·Log(N) Method for Ewald Sums in Large Systems. Journal of Chemical Physics 98, 10089-10092 (1993).
J. D. Hunter, Matplotlib: A 2D graphics environment. Comput Sci Eng 9, 90-95 (2007).
H. Hubner et al., Structure-guided development of heterodimer-selective GPCR ligands. Nat Commun 7, 12298 (2016).
B. T. DeVree et al., Allosteric coupling from G protein to the agonist-binding pocket in GPCRs. Nature 535, 182-186 (2016).
L. Vedel, H. Brauner-Osborne, J. M. Mathiesen, A cAMP Biosensor-Based High-Throughput Screening Assay for Identification of Gs-Coupled GPCR Ligands and Phosphodiesterase Inhibitors. J Biomol Screen 20, 849-857 (2015).
T. H. Althuis, H. J. Hess, Synthesis and identification of the major metabolites of prazosin formed in dog and rat. J Med Chem 20, 146-149 (1977).
L. Zhang et al., Synthesis and biological activities of quinazoline derivatives with ortho-phenol-quaternary ammonium salt groups. Bioorg Med Chem 15, 6920-6926 (2007).
E. F. DiMauro et al., Discovery of aminoquinazolines as potent, orally bioavailable inhibitors of Lck: synthesis, SAR, and in vivo anti-inflammatory activity. J Med Chem 49, 5671-5686 (2006).
Y. Cheng, W. H. Prusoff, Relationship between the inhibition constant (K1) and the concentration of inhibitor which causes 50 percent inhibition (150) of an enzymatic reaction. Biochem Pharmacol 22, 3099-3108 (1973).

### Formal Sequence Listing

Portion of human β2AR
(SEQ ID NO: 1)
GNFWCEFWTSIDVLCVTASIETLCVIAVDRYFAITS
Portion of human β2AR
(SEQ ID NO: 2)
NQAYAIASSIVSFYVPLVIMVFVYSRVFQEAKRQLQ
KIDKSE
Portion of mouse β1AR
(SEQ ID NO: 3)
GSFFCELWTSVDVLCVTASIETLCVIALDRYLAITS
Portion of mouse β1AR
(SEQ ID NO: 4)
NRAYAIASSVVSFYVPLCIMAFVYLRVFREAQKQVKKIDS
Portion of human α1AR
(SEQ ID NO: 5)
GRVFCNIWAAVDVLCCTASIIVIGLCIISIDRYIGVSY
Portion of human α1AR
(SEQ ID NO: 6)
EPGYVLFSALGSFYLPLAIILVMYCRVYVVAKRESRG
LKSGL
Portion of mouse α2AR
(SEQ ID NO: 7)
GKVWCEIYLALDVLFCTSSIVHLCAISLDRYWSITQ
Portion of mouse α2AR
(SEQ ID NO: 8)
QKWYVISSSIGSFFAPCLIIVIILVYVRIYQIAKRR
TRVPPSR
Portion of human 5HT1AR
(SEQ ID NO: 9)
GQVTCDLFIALDVLCCTSSILHLCAIALDRYWAITD
Portion of human 5HT1AR
(SEQ ID NO: 10)
DHGYTIYSTFGAFYIPLLLMLVLYGRIFRAARFRIR
KTVKKV
Portion of human M2R
(SEQ ID NO: 11)
GPVVCDLWLALDYVVSNASVMNLLIISFDRYFCVTK
Portion of human M2R
(SEQ ID NO: 12)
NAAVTFGTAIAAFYLPVIIMTVLYWHISRASKSRIKKDKKE
Portion of human M3R
(SEQ ID NO: 13)
GNLACDLWLAIDYVASNASVMNLLVISFDRYFSITR
Portion of human M3R
(SEQ ID NO: 14)
EPTITFGTAIAAFYMPVTIMTILYWRIYKETEKRTKELAGL
Portion of human D2R
(SEQ ID NO: 15)
SRIHCDIFVTLDVMMCTASILNLCAISIDRYTAVAM
Portion of human D2R
(SEQ ID NO: 16)
NPAFVVYSSIVSFYVPFIVTLLVYIKIYIVLRRRRKRVNTK
Portion of human NTS1R
(SEQ ID NO: 17)
GDAGCRGYYFLRDACTYATALNVASLSVERYLAICH
Portion of human NTS1R
(SEQ ID NO: 18)
TATVKVVIQVNTFMSFIFPMVVISVLNTIIANKLTV
MVRQAAEQG
Portion of human δOR
(SEQ ID NO: 19)
GELLCKAVLSIDYYNMFTSIFTLTMMSVDRYIAVCH
Portion of human δR
(SEQ ID NO: 20)
SWYWDTVTKICVFLFAFVVPILIITVCYGLMLLRLRSV
Portion of human κOR
(SEQ ID NO: 21)
GDVLCKIVISIDYYNMFTSIFTLTMMSVDRYIAVCH
Portion of human κOR
(SEQ ID NO: 22)
YSWWDLFMKICVFIFAFVIPVLIIIVCYTLMILRLKSV
Portion of human μOR
(SEQ ID NO: 23)
GTILCKIVISIDYYNMFTSIFTLCTMSVDRYIAVCH
Portion of human μOR
(SEQ ID NO: 24)
TWYWENLLKICVFIFAFIMPVLIITVCYGLMILRLKSV
Portion of human PAR2
(SEQ ID NO: 25)
GEALCNVLIGFFYGNMYCSILFMTCLSVQRYWVIVN
Portion of human PAR2
(SEQ ID NO: 26)
LVGDMFNYFLSLAIGVFLFPAFLTASAYVLMIRMLRSS
Portion of human β2AR
(SEQ ID NO: 27)
TASIETLCVIAVDRYFAITS
Portion of human β2AR
(SEQ ID NO: 28)
NQAYAIASSIVSFYVPLVIMVFV
Portion of mouse β1AR
(SEQ ID NO: 29)
TASIETLCVIALDRYLAITS
Portion of mouse β1AR
(SEQ ID NO: 30)
NRAYAIASSVVSFYVPLCIMAF
Portion of human α1AR
(SEQ ID NO: 31)
TASIMGLCIISIDRYIGVSY
Portion of human α1AR
(SEQ ID NO: 32)
EPGYVLFSALGSFYLPLAIILV
Portion of mouse α2AR
(SEQ ID NO: 33)
TSSIVHLCAISLDRYWSITQ
Portion of mouse α2AR
(SEQ ID NO: 34)
QKWYVISSSIGSFFAPCLIIVIIL
Portion of human 5HT1AR
(SEQ ID NO: 35)
TSSILHLCAIALDRYWAITD
Portion of human 5HT1AR
(SEQ ID NO: 36)
DHGYTIYSTFGAFYIPLLLMLV
Portion of human M2R
(SEQ ID NO: 37)
NASVMNLLIISFDRYFCVTK
Portion of human M2R
(SEQ ID NO: 38)
NAAVTFGTAIAAFYLPVIIMTV
Portion of human M3R
(SEQ ID NO: 39)
NASVMNLLVISFDRYFSITR
Portion of human M3R
(SEQ ID NO: 40)
EPTITFGTAIAAFYMPVTIMTI
Portion of human D2R
(SEQ ID NO: 41)
TASILNLCAISIDRYTAVAM
Portion of human D2R
(SEQ ID NO: 42)
NPAFVVYSSIVSFYVPFIVTLL
Portion of human NTS1R
(SEQ ID NO: 43)
YATALNVASLSVERYLAICH
Portion of human NTS1R
(SEQ ID NO: 44)
TATVKVVIQVNTFMSFIFPMVVISV
Portion of human δOR
(SEQ ID NO: 45)
FTSIFTLTMMSVDRYIAVCH
Portion of human δOR
(SEQ ID NO: 46)
SWYWDTVTKICVFLFAFVVPILIITV
Portion of human κOR
(SEQ ID NO: 47)
FTSIFTLTMMSVDRYIAVCH
Portion of human κOR
(SEQ ID NO: 48)
YSWWDLFMKICVFIFAFVIPVLIIIV
Portion of human μOR
(SEQ ID NO: 49)
FTSIFTLCTMSVDRYIAVCH
Portion of human μOR
(SEQ ID NO: 50)
TWYWENLLKICVFIFAFIMPVLIITV
Portion of human PAR2
(SEQ ID NO: 51)
YCSILFMTCLSVQRYWVIVN
Portion of human PAR2
(SEQ ID NO: 52)
LVGDMFNYFLSLAIGVFLFPAFLTAS

1. A compound having the formula: 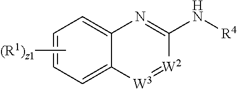
wherein
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
W² is N, CH, or C(R²);
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W³ is N, CH, or C(R³);
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently substituted or unsubstituted aryl, substituted or unsubstituted heteroaryl, substituted or unsubstituted cycloalkyl, substituted or unsubstituted spirocycloalkyl, substituted or unsubstituted heterocycloalkyl, hydrogen, substituted or unsubstituted alkyl, or substituted or unsubstituted heteroalkyl;
R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl; and R^{3A} and R^{3B} substituents bonded to the same nitrogen atom may optionally be joined to form a substituted or unsubstituted heterocycloalkyl or substituted or unsubstituted heteroaryl;
X, X¹, X², and X³ are independently —F, —Cl, —Br, or —I;
n1, n2, and n3 are independently an integer from 0 to 4; and
m1, m2, m3, v1, v2, and v3 are independently 1 or 2.

2. A compound having the formula: 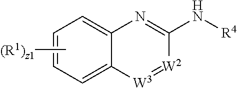
wherein
R¹ is independently halogen, —CX¹₃, —CHX¹₂, —CH₂X¹, —OCX¹—, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
W² is N, CH, or C(R²);
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W³ is N, CH, or C(R³);
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently substituted or unsubstituted aryl, substituted or unsubstituted heteroaryl, substituted or unsubstituted cycloalkyl, substituted or unsubstituted spirocycloalkyl, substituted or unsubstituted heterocycloalkyl, hydrogen, or substituted or unsubstituted alkyl;
R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; and R^{3A} and R^{3B} substituents bonded to the same nitrogen atom may optionally be joined to form a substituted or unsubstituted heterocycloalkyl or substituted or unsubstituted heteroaryl;
X, X¹, X², and X³ are independently —F, —Cl, —Br, or —I;
n1, n2, and n3 are independently an integer from 0 to 4; and
m1, m2, m3, v1, v2, and v3 are independently 1 or 2.

3. The compound of claim 1, wherein R⁴ is substituted or unsubstituted phenyl, substituted or unsubstituted naphthyl, substituted or unsubstituted pyridinyl, or substituted or unsubstituted pyrimidinyl.

4. The compound of claim 1, having the formula: 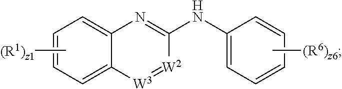
wherein
R⁶ is independently halogen, —CX⁶₃, —CHX⁶₂, —CH₂X⁶, —OCX⁶₃, —OCH₂X⁶, —OCHX⁶₂, —CN, —SOₙ₃R^{6D}, —SOᵥ₃NR^{6A}R^{6B}, —NHC(O)NR^{6A}R^{6B}, —N(O)ₘ₃, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 5;
R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
X⁶ is independently —F, —Cl, —Br, or —I;
n6 is independently an integer from 0 to 4; and
m6 and v6 are independently 1 or 2.

5. The compound of claim 1, wherein W² is N, and W³ is C(R³).

6. (canceled)

7. (canceled)

8. The compound of claim 1, wherein R³ is independently —NH₂, —OH, —O-alkyl, —N-alkyl, —N-cycloalkyl, —N-dialkyl, unsubstituted C₁-C₄ alkyl, —CN, —CF₃, —NO₂, —COOH, or —NHC(═NH)NH₂.

9. The compound of claim 1, wherein R³ is independently —NH₂.

10. The compound of claim 1, wherein z1 is 1.

11. The compound of claim 4, having the formula:

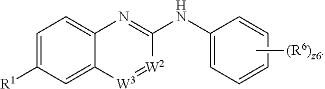

12. (canceled)

13. The compound of claim 1, wherein R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, unsubstituted C₁-C₄ alkyl, unsubstituted phenyl, or unsubstituted 5 to 6 membered heteroaryl.

14. (canceled)

15. The compound of claim 1, wherein R¹ is independently halogen or —CF₃.

16. (canceled)

17. (canceled)

18. The compound of claim 4, wherein R⁶ is independently —CH₂OH, —CH₂CH₂COOH, —CH₂CH₂COOCH₂CH(OH)CH₂OH, —SO₂NH₂, —C(O)NHCH₃, —C(O)CH₃, —C(O)OCH₃, or —OH.

19. The compound of claim 1, wherein z6 is 1 or 0.

20. (canceled)

21. The compound of claim 1, having the formula:

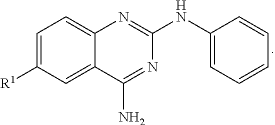

22. The compound of claim 1, having the formula:

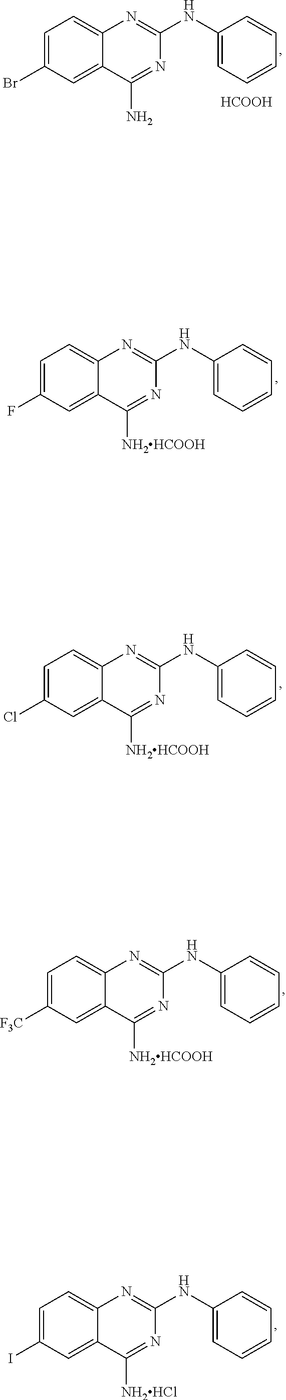

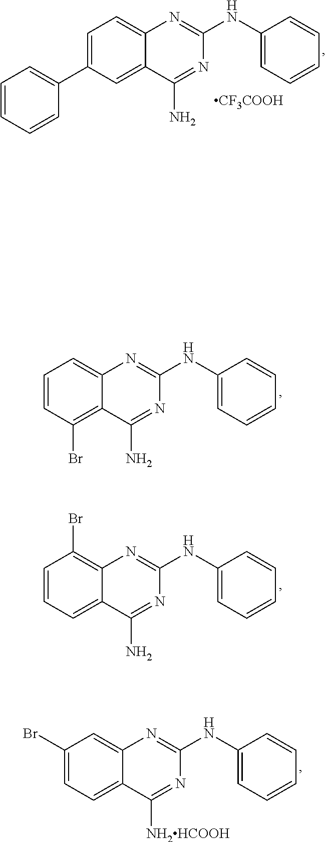

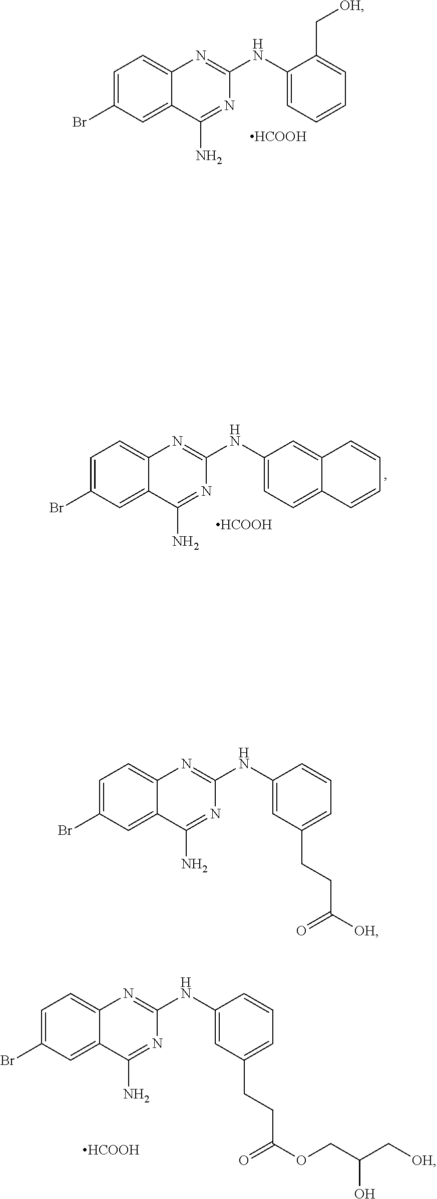

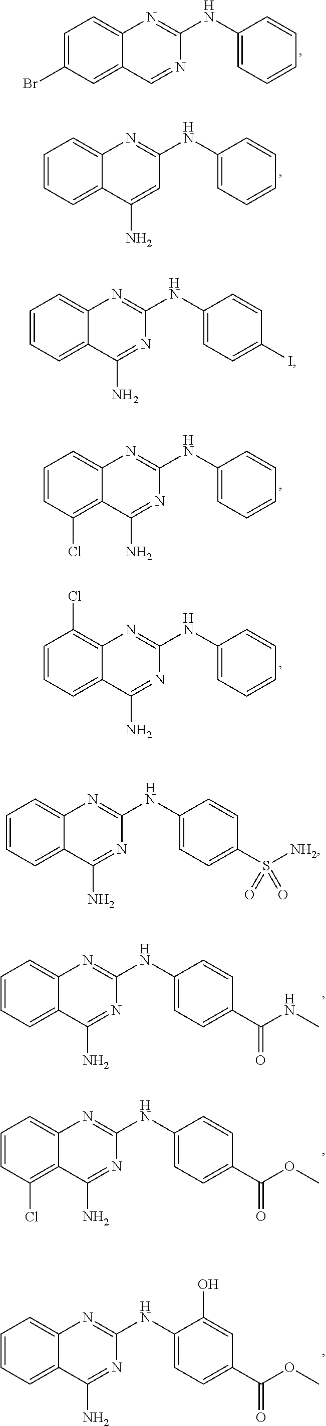

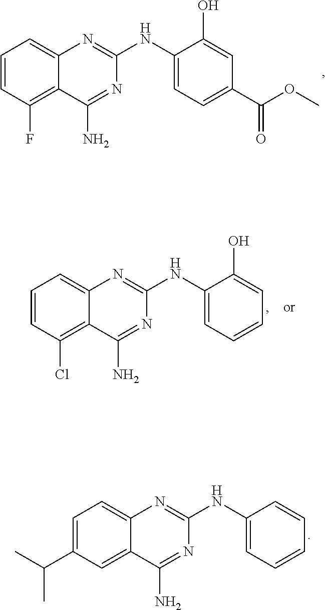

23. A pharmaceutical composition comprising a compound of claim 1 and a pharmaceutically acceptable excipient.

24. The pharmaceutical composition of claim 23, further comprising a second agent, wherein the second agent is a β2 adrenergic receptor inhibitor.

25. A method of treating a disease associated with β2 adrenergic receptor, said method comprising administering to a subject in need thereof a therapeutically effective amount of a compound of claim 1.

26. A method of treating Parkinson's disease, hypertension, heart failure, asthma, myocardial infarction, angina pectoris, tachycardia, anxiety, tremor, migraine headache, cluster headache, hyperhidrosis, glaucoma, thyrotoxicosis, hyperthyroidism, esophageal variceal, ascites, post-traumatic stress disorder, psychogenic polydispsia, hemangioma, or cardiomyopathy, said method comprising administering to a subject in need thereof a therapeutically effective amount of a compound of claim 1.

27. The method of claim 25, further comprising administering a second agent to the subject in need thereof, wherein the second agent is a β2 adrenergic receptor inhibitor.

28. The method of claim 26, further comprising administering a second agent to the subject in need thereof, wherein the second agent is a β2 adrenergic receptor inhibitor.
